(19) United States
(12) Patent Application Publication
(10) Pub. No.: US 2026/0139244 A1
(43) Pub. Date: May 21, 2026

(54) REAGENTS AND METHODS FOR CELL IDENTIFICATION AND CHARACTERIZATION

(71) Applicant: CELL ORIGINS, LLC, Columbia, MO (US)

(72) Inventors: Jessica Newton Northup, Boonville, MO (US); Mette Soendergaard, Macomb, IL (US)

(21) Appl. No.: 18/875,370

(22) PCT Filed: Jun. 14, 2023
(86) PCT No.: PCT/US2023/025326

§ 371 Date: Dec. 16, 2024

Related U.S. Application Data
(60) Provisional application No. 63/352,460, filed on Jun. 15, 2022.

(51) Int. Cl. C12N 15/10; C07K 7/08; C12N 15/86

(52) U.S. Cl. CPC C12N 15/1037; C07K 7/08; C12N 15/86

(57) ABSTRACT

Disclosed are peptides that differentially bind to analytes, and methods for obtaining the peptides. Disclosed are methods for determining binding of peptides to analytes by detecting nucleic acids that are associated with the peptides. Disclosed are methods for propagation and/or screening for the peptides by reconstruction of viruses encoding the peptides from nucleotide sequence information. Disclosed are kits containing reagents for performing the methods disclosed

This application claims the benefit of and priority to U.S. Provisional Application No. 63/352,460, filed on Jun. 15, 2022, the entire content of which is incorporated herein by reference in its entirety.

### GOVERNMENT INTERESTS

This invention was made with government support under grant IR43GM131450-01 A1 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

Aspects of the invention are drawn to reagents and methods for identifying and characterizing cells.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### BACKGROUND OF THE INVENTION

Phenotypic drift of cells and cell lines grown in vitro can result in cells that have unexpected properties and/or do not have expected properties. The presence of unexpected properties or absence of expected properties in cells can compromise data obtained from experiments using the cells.

### SUMMARY OF THE INVENTION

Disclosed are methods for assaying peptide binding to a biological particle, including a cell, cell line, tissue, and the like. In some embodiments, the methods can detect and quantify a molecule (e.g., nucleic acid) associated with a bound peptide as a proxy for the bound peptide. Disclosed are methods for identifying peptides that differentially bind to a biological particle depending on a condition or state of the particle (e.g., cell health). Disclosed are peptides that differentially bind to a biological particle depending on a condition or state of the particle. Disclosed are bacteriophages, phagemids, plasmids, and the like that encode the peptides. Disclosed are kits that contain the peptides, bacteriophages, phagemids, plasmids and the like. Disclosed are methods for reconstructing bacteriophage or phagemid clones from a library.

In some embodiments, a method for assaying binding of a peptide can include contacting a peptide or polypeptide with a second molecule (e.g., an analyte, a cell containing or displaying an analyte) to which the peptide or polypeptide can bind. The method can include quantifying binding of the peptide or polypeptide by detecting a nucleic acid molecule associated with the peptide or polypeptide. In some embodiments, the peptide or polypeptide can be displayed on a surface of a virus (e.g., bacteriophage, phagemid particle) In some embodiments, the nucleic acid molecule detected can be a part of a genome of the virus. In some embodiments, the nucleic acid can be detected by polymerase chain reaction (PCR).

In some embodiments, methods for identifying a peptide that differentially binds to a biological particle can include screening a bacteriophage or phagemid particle library for clones that saturably bind to a biological particle under a first condition and identifying the clones that have increased or decreased binding to the biological particle under a second condition. The conditions can include identity of the biological particle or cell, tissue type from which a cell is derived, cell age, cell passage number, cell density, media composition in which the cell is propagated, culture conditions, plating efficiency, cell growth or proliferation rate, cell viability, cell metabolism, and the like. In some embodiments, differential peptide binding can inform as to the condition of a cell (e.g., cell health). In some embodiments, differential peptide binding can be diagnostic of a cell condition (e.g., cancer).

In some embodiments, peptides that differentially bind to a biological particle depending on a condition or state of the particle can include AVAGLFTGPQVDTVV (SEQ ID NO: 1); HHFLFPSFVWAVAYS (SEQ ID NO: 2): YYVGFGPLRVVRSVE (SEQ ID NO: 3): TSRASWCCAVVVDSL (SEQ ID NO:4); or AGATGYRYGSPKTRF (SEQ ID NO: 5), or peptides at least 90% identical thereto.

In some embodiments, methods for reconstructing bacteriophage or phagemid clones encoding a foreign peptide, from a bacteriophage or phagemid library, can include obtaining a nucleotide sequence of a genome segment of the bacteriophage or phagemid that encodes the foreign peptide, incorporating the nucleotide sequence of the genome segment into a bacteriophage or phagemid genome to reconstitute the genome of the bacteriophage or phagemid that encodes the genome segment, and introducing the reconstituted genome into a cell to produce a reconstituted bacteriophage or phagemid particle.

### BRIEF DESCRIPTION OF THE DRAWINGS

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1 is a schematic diagram supporting the concept that efficient cell line characterization and/or identification can require multiple forms of investigation. There can be many points of potential difference between tissue culture cell lines between laboratories. Thus, cell line identification can require protocols able to probe multiple elements of cell biology. (A) STR analysis can probe DNA status. While gene expression can be probed via (B) qRT-PCR to quantify RNA levels within the cell. Expression of cell surface biomarkers can be probed by (C) qPCR of the biomarker targeting phage clones bound to the surface of the cell. The three methods can be used in combination to give an overall view of cell health.

FIG. 2 illustrates phenotypic characterization of a cell line using a proposed kit. (A) describes use of phage clones (each with a unique DNA sequence highlighted in a distinct shape and color) incubated with cells that might be contaminated (left side of dish) or cultured for too long (right side of dish). A normal, desired cell expressing the biomarker compatible with the triangle-shaped phage clone (blue biomarker) is pictured, far left. Contaminating cells would likely express a different biomarker or might be distinct because of the absence of blue biomarker. In the drawing, over-cultured cells begin to overexpress the blue biomarker. Another possibility might be that the blue biomarker and associated RNA may remain the same, but a new oval-shaped, yellow RNA and biomarker are expressed. (B) represents the removal of excess phage and phage with no available biomarker. The final cell surface bound phage and the tissue culture cells are then processed, and the total RNA and DNA purified. (C) illustrates use of the unique phage sequences along with their matched RNA transcripts for templates within a qPCR/qRT-PCR protocol.

FIG. 3. Comparison of multiple parallel phage display selections. All peptide sequences identified within the negative selection (wells with media, but no cells) were deleted from all other sequences found in experimental selections. Further controls included comparison of LNCaP to HEK-293 binding/selection. This was done to verify that the selection process was not taken over by nonspecific peptide sequences and/or phage clones with growth advantages. (A) Selection 1 against low passage number LNCaP cells-Comparing different Phage Selection Methods; (B) Selection 1 against low passage number-Comparing phage sequences found on LNCaP versus HEK293; (C) Selection 2 against high passage number LNCaP cells-Comparing different Phage Selection Methods; (D) Selection 2 against high passage number-Comparing phage sequences found on LNCaP versus HEK293; (E) Comparison of Round 4 LNCaP specific phage clone sequences from Selection 1 (low passage number) versus Selection 2 (high passage number); (F) Comparison of Round 4 HEK-293 specific phage clone sequences from Selection 1 (low passage number) versus Selection 2 (high passage number).

FIG. 4 shows a table related to use and comparison of different quantification methods of cell surface bound phage. Fixed LNCaP and HEK293 cells in 6-well plates were incubated with 1E11 v/mL for 1 hr at room temperature, washed, eluted and quantified via qPCR (f88=no targeting peptide).

FIG. 5 shows a table related to use and comparison of different elution strategies. Fixed LNCaP and HEK293 cells in 6 well plates were incubated with 1E11 v/mL for 1 hr at room temperature, washed, eluted (using 0.1N HCl-pH2.2, 2.5% CHAPS detergent, or 1.25% trypsin), and quantified via qPCR. (f88=no targeting peptide).

FIG. 6 shows EC50 values (v/mL) for phage display selected phage clones against LNCaP and HEK293 cells. Serial dilutions of phage clones were incubated with fixed cells, washed, and trypsin eluted. The concentrations of eluted phage were then determined via qPCR and EC50 values, reported in v/mL, calculated using Prism 7 software (GraphPad Software, La Jolla, California).

FIG. 7. Cell binding of phage clones 44463 and 44465 to LNCap cells of increasing age. Frozen aliquots of LNCaP cells at passage number 10, 20, 30, and 40 were defrosted and expanded for cell binding assays. Serial dilutions of phage were incubated with fixed cells, washed, trypsin eluted and quantified via qPCR.

FIG. 8. Changes in cell surface binding of phage clones in response to confluency of cells. LNCaP cells were grown to different confluencies (10, 50, 70, 80, 100 and 150% confluence). The cell numbers were estimated using Deep Red Cell Mask (FisherSci).

FIG. 9 Cluster of functionally related genes identified in the loss of binding assay with p30-1 positive control phage clone. Legend for the connecting lines: Blue=from curated databases; Pink=experimentally determined; Green=predicted from gene neighborhood; Red=predicted from gene fusions; Purple=predicted from gene co-occurrence; Yellow=textmining; Black=co-expression; Light Purple=protein homology

FIG. 10 Cluster of functionally related genes identified in the loss of binding assay with the experimental phage clone, 44463. Legend for the connecting lines: Blue=from curated databases; Pink=experimentally determined; Green=predicted from gene neighborhood; Red=predicted from gene fusions; Purple=predicted from gene co-occurrence; Yellow=textmining; Black=co-expression; Light Purple=protein homology.

FIG. 11 Cluster of functionally related genes identified in the loss of binding assay with the experimental phage clone, 44465. Legend for the connecting lines. Blue=from curated databases; Pink=experimentally determined; Green=predicted from gene neighborhood; Red=predicted from gene fusions; Purple=predicted from gene co-occurrence; Yellow=textmining; Black=co-expression; Light Purple=protein homology.

FIG. 12 is a schematic diagram illustrating an example method for reconstruction of a bacteriophage or phagemid clone encoding a foreign peptide from a library, using the nucleotide sequence encoding the foreign peptide.

FIG. 13 Comparison of phage clone binding to the surfaces of three different cell lines. Low passage number LNCaP, PC3, and HEK-293 cells were grown to 80% confluency, fixed, rinsed, incubated with phage, washed, and cells stained with Cell Mask. Phage were then trypsin eluted and quantified via qPCR. The reported data is normalized by Cell Mask values (AU).

FIG. 14 illustrates example Measures of Cell Health, as described herein, and example methods for their measurement.

FIG. 15 illustrates example biomarker expression levels within human versus mouse cell lines. Illustrated are example changes in cell surface binding of phage to biomarkers on various cultured cell lines. The data show ratios of phage clone binding to negative-control phage clone binding.

FIG. 16 illustrates example changes in biomarker expression levels within human cell lines due to growth and maintenance of cells in different media formulations. Changes in cell surface binding of phage to biomarkers on various cultured cell lines. First, cells were defrosted and maintained within the ATCC specified media (Reg Media) or an alternative media formulation (Alt Media). The cell surface binding of the phage clones was probed when the cells were in these media. The data show ratios of phage clone binding to negative-control phage clone binding.

FIG. 17 illustrates example differential gene expression in old versus young LNCaP cells. RNAseq analysis of biological replicates showed about 455 differentially expressed genes. About 337 genes were up-regulated (Red, right panel), while about 118 genes were down-regulated (Blue, left panel) as determined by the filtering criteria. Specifically, Log 2 FC<=−1 or Log 2 FC>=1 and FDR<0.05.

FIG. 18 illustrates the top eight cellular pathways with significant changes in gene expression in old LNCaP cells as compared to young LNCAP cells (based on P Value of 0.0026 or less).

### DETAILED DESCRIPTION OF THE INVENTION

Disclosed herein are reagents and methods for identifying and/or characterizing cells. In some embodiments, the reagents and methods can determine a phenotypic profile of a cell or cell line.

In some embodiments, the reagents disclosed here include peptides that differentially bind to a cell or cell line depending on a condition of the cell or line. In some embodiments, a peptide may bind to a cell line, for example, if the cell line has been passaged a low number of times in vitro. The peptide may not bind or may bind at a reduced level if the cell line has been passaged a high number of times. Other peptides may differentially bind to cells or cell lines dependent on different factors. In some embodiments, a peptide may differentially bind a cell based, for example, on identity of the cell, the tissue type from which the cell was derived, cell density in vitro, composition of the medium in which the cells are grown, other culture conditions, plating efficiency of the cells, growth or proliferation rate, viability, cellular metabolism, production of reactive oxygen species, mitochondrial membrane potential, and the like. Binding data for a combination of these peptides may be indicative of a phenotypic profile of the cells. Disclosed herein are such peptides and methods for identifying and obtaining these peptides. Disclosed also herein are methods to identify a cellular gene associated with expression of the peptides on the cell.

While not wishing to be bound by theory, the peptides generally bind to a specific molecule or molecules on a surface of the cell (i.e., a cellular analyte). Generally, binding of the peptides to the analytes is saturable, meaning that once enough peptide is contacted with the cell such that all of the analytes on the cell surface are bound, no additional peptides will bind to the cell. Generally, the amount of peptide bound to a cell at saturation positively correlates with the amount of the analyte on the cell surface. Therefore, saturable binding of the peptides disclosed herein can be used to determine an amount or level of an analyte in a cell.

The disclosed methods for obtaining a phenotypic profile of a cell or line can include methods for binding the peptides to cells and quantifying the binding. In some embodiments the peptides can be displayed on the surface of a particle and binding of the particle to a cell can be used to quantify peptide binding. For example, the particle can be a virus, including a bacteriophage or phagemid, which displays the peptide on the virus surface, in some embodiments as a fusion with a viral coat protein. In some embodiments, binding of the virus to a cell can be quantified by measuring the amount of viral genome from the cell-bound virus. In some embodiments, quantitative polymerase chain reaction (qPCR) can be used to quantify the amount of the viral genome.

In some embodiments, the disclosed methods for determining an amount of an analyte in a cell, obtained using peptide binding to the cell, as described above, can be coupled with methods that determine an amount of mRNA that encodes the analyte or is related to expression of the analyte in the cell. In some embodiments, the methods for determining amount of mRNA may include real-time quantitative reverse transcription polymerase chain reaction (qRT-PCR). Relationships between the amount of a cell analyte (as determined by qPCR of virus binding) with the amount of an mRNA encoding the analyte (as determined by qRT-PCR of the mRNA) can provide insights into cell health.

Also disclosed herein are kits for determining a phenotypic profile of a cell. The phenotypic profile can be indicative of cell health. In some embodiments, the kits may include one or more peptides, viruses expressing the peptides, and/or genomes of the virus that encode the peptides, as described above.

Also disclosed here are methods for reconstructing a bacteriophage or phagemid clone that encodes a foreign peptide, the clone generally obtained by screening a bacteriophage/phagemid library, by obtaining the nucleotide sequence encoding the foreign peptide from the bacteriophage or phagemid genome, cloning the sequence into a genome to reconstitute the clone's genome, then introducing the reconstituted genome into a host bacterial cell that can support replication of the bacteriophage/phagemid, and reproduce the bacteriophage/phagemid.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Herein, “analyte” refers to a molecule or collection of associated molecules, like proteins, lipids, carbohydrates, glycoproteins and the like. “Cellular” analytes include biomolecules from cells.

Herein, “associated with” generally refers to being in close proximity to. “Associated with” can be used herein to refer to the proximity of a peptide and nucleic acid. In some embodiments, associated with can refer to a virus particle having a recombinant peptide displayed on the viral particle's surface and a genome of the virus inside the viral particle. In this example, the genome and peptide can be said to be associated with one another.

Herein, “bacteriophage”, “phage” or “o” is a virus that infects prokaryotic cells, including bacteria.

Herein, “Ct” refers to threshold cycle in real-time PCR (also called quantitative PCR or qPCR), which is a relative measurement of a concentration of a target in the PCR reaction.

Herein, differentially” refers to something that is variable depending on certain conditions. Herein. “differentially” can refer to peptide binding to a cellular analyte based on a condition or state of the cell.

Herein, “display” refers to show or to make prominent. Herein, “display” can be used to refer to a peptide that is configured on a surface of a virus such that the peptide can bind to a cellular analyte.

Herein, “drift” refers to changes in a cell, for example, over time or based on conditions to which the cell is subjected. For example, “drift” can refer to biochemical changes resulting from epigenetic, transcriptional and/or translational changes, or changes to post-translational modification or processing,

Herein, “marker” can refer to a cellular analyte that is indicative of a condition or state of a cell.

Herein, “peptide-nucleic acid hybrid” can refer to a nucleic acid associated with a peptide that is not part of a virus.

Herein. “phagemid” refers to a genome that has properties of both bacteriophages and bacterial plasmids. Generally, these genomes can contain both a bacteriophage and plasmid origin of replication. A “phagemid particle” can refer to a bacteriophage containing a phagemid as its genome.

Herein, “phenotypic profile” refers to a combination of particular characteristics and/or properties of a cell. Herein, detection of cellular analytes, generally through differential binding of peptides to the analytes, can indicate a phenotypic profile.

### Peptide Binding to Cells as Indicators of Cell Phenotypes

Disclosed here are peptides that bind to cells. In some embodiments, the peptides differentially bind to cells (e.g., the peptides bind to specific cells, but not others). In some embodiments, binding or non-binding of the peptide to cells is indicative of a certain condition or state of the cell.

Generally, the peptides that bind to cells can bind specifically to molecules or analytes on, in or of the cell. In some embodiments, the peptides bind to an analyte on the exterior surface of the cell (e.g., in or on the cell membrane). “Specific” binding generally means that the peptide can bind to one analyte or to a family of related analytes, but does not bind to other, unrelated analytes. In some embodiments, the peptides can bind to molecules or analytes on the surface of a cell. In some embodiments, the peptides can bind to analytes that are biomolecules like proteins, lipids, carbohydrates, glycoproteins, and the like. In some embodiments, the peptides can bind to cellular receptors, ligands, cluster of differentiation (CD) molecules, MHC molecules, tumor antigens, and the like. In some embodiments, the peptides can bind to molecules involved in neuronal guidance or axon outgrowth, peroxisome synthesis, metabolism of fatty acids/lipids, regulation of transcription e.g., ligand-activated transcription factors), and the like.

Binding of the peptides to cellular analytes generally is saturable. Saturable binding means that the amount of peptide that a cell can bind is limited (e.g., as an increasing amount of peptide is contacted with a cell, at some point no more peptides can be bound by the cell). Saturable binding generally depends on and is indicative of the amount of a cellular analyte on a cell surface, for example, to which the peptide can bind. While not wishing to be bound by theory, at saturation, all analytes on a cell to which the peptide can bind have bound peptide. Generally, when binding of these peptides to a cell is saturable, the amount of the peptide bound to the cell at saturation positively correlates with the amount of analyte on the cell surface.

In some embodiments, the peptides can be 4, 5, 6, 7, 8, 9, 10, 11, 12, 13, 14, 15, 16, 17, 18, 19, 20, 21, 22, 23, 24, 25, 26, 27, 28, 29 or 30 amino acids in length. In some embodiments, the peptides can be between about 4 and 35, 5 and 33, 6 and 31, 7 and 29, 8 and 27, 9 and 25 or 10 and 23 amino acids in length. Generally, the peptides include amino acids. In some embodiments, any amino acid can be used. In some embodiments, the peptides can include the 20 essential and nonessential amino acids. Generally, the amino acids in the peptides are L enantiomers.

In some embodiments, the peptides can be as follows:

(SEQ ID NO: 1)
AVAGLFTGPQVDTVV
(SEQ ID NO: 2)
HHFLFPSFVWAVAYS;
(SEQ ID NO: 3)
YYVGFGPLRVVRSVE;
(SEQ ID NO: 4)
TSRASWCCAVVVDSL;
and
(SEQ ID NO: 5)
AGATGYRYGSPKTRF.

In embodiments, the peptides having amino acid sequences at least 85, 86, 87, 88, 89, 90, 91, 92, 93, 94, 95, 96, 97, 98 or 99 percent identical to the sequences of SEQ ID NOs. 1-5. The amino acid sequences that make up the the peptides can be part of larger molecules, like polypeptides or proteins.

Herein, in some embodiments, the peptides are those that differentially bind to cells. In some embodiments, differential binding of a peptide refers to a peptide binding to one cell and binding to a lesser or greater degree to another cell. In some embodiments, a peptide binds to a first cell and does not bind to a second cell. In some embodiments, differential binding of a peptide refers to a peptide binding to a cell under one condition or state, or a set of conditions or states, and binding to a lesser or greater degree to that cell, or a related cell, when the cell is in or has a second set of conditions or states. While not wishing to be bound by theory, differential binding of a peptide can be interpreted to mean that the analyte to which the peptide can specifically bind is present when the peptide binds to a cell, and that the analyte is not present when the peptide does not bind to the cell. While not wishing to be bound by theory, the extent to which a peptide binds to a cell positively correlates with the number of analytes on the cell.

In some embodiments, a peptide may differentially bind to a cell based on identity of the cell, tissue from which the cell originated, cell age, cell passage number, cell density, media composition in which the cell is propagated, culture conditions and the like. In some embodiments, a peptide may differentially bind to a cell based on plating efficiency of the cell, growth or proliferation rate, cell viability, cellular metabolism including citrate metabolism or triclyceride metabolism, oxygen consumption and/or extracellular acidification rate, production of or sensitivity to reactive oxygen species (ROS), mitochondrial membrane potential, and the like. Peptides can differentially bind to cells based on other cellular properties or conditions imposed on the cells. In some embodiments, a combination of these properties or conditions can be considered a measure of cell health (MCH).

In some embodiments, a peptide can bind to a cell if the cell has a property, state or condition and cannot bind or can bind less to the cell if the cell does not have or has less of the property, state or condition, or has less of the property. In some embodiments, a peptide can bind to a cell if the cell does not have a property, state or condition and can bind if the cell has the property.

In some embodiments, a peptide can bind to a cell when the cell has been passaged fewer times in culture and cannot bind or can bind less when a cell has been passaged many times in culture. In some embodiments, a peptide cannot bind to a cell when the cell has been passaged fewer times in culture and can bind to the cell when the cell has been passaged many times in culture.

In some embodiments, a peptide can bind to a cell when the cell is sub-confluent on a surface to which it is attached in vitro and cannot bind or can bind less to the cell when the cell is confluent on the surface. In some embodiments, a peptide cannot bind to a cell when the cell is sub-confluent on a surface when grown in vitro and can bind to the cell when the cell is confluent on the surface.

While not wishing to be bound by theory, differential binding of a peptide to a cell under example conditions like these can be interpreted to mean that the analyte to which a peptide binds is present on a cell under conditions or circumstances in which the peptide binds to the cell (e.g., cells at a low passage number in vitro: low confluency of cells that grow attached to a surface in vitro, low density cells in suspension) and is not present, or is present at lower amounts, under conditions or circumstances in which the peptide does not bind to the cell (e.g., cells at a high passage number in vitro: high confluency of cells that grow attached to a surface in vitro, high density cells in suspension).

In some embodiments, differential binding of a peptide to a cell under example conditions like these can be interpreted to mean that the analyte to which a peptide binds is not present on a cell under conditions or circumstances in which the peptide does not bind to the cell (e.g., cells at a low passage number in vitro; low confluency of cells that grow attached to a surface in vitro) and is present under conditions or circumstances in which the peptide binds to the cell (e.g., cells at a high passage number in vitro; high confluency of cells that grow attached to a surface in vitro).

In some embodiments, one or more cellular conditions, states, and the like, as detected using peptide binding/peptide non-binding, can be referred to a cell phenotype.

In some embodiments, differential binding of a peptide to a cell can depend on a cell's type (e.g., epithelial vs. muscle), the species of origin of a cell, the composition of a medium, and the like.

In some embodiments, peptides having the amino acid sequence of SEQ ID NO: 3 or SEQ ID NO: 4 can bind to cells at a relatively low passage number (e.g., passage number≤30) and bind less well at a relatively high passage number (e.g., passage number≥40). In some embodiments, the cells are prostate cells. In some embodiments, the cells are LNCaP cells.

In some embodiments, peptides having the amino acid sequence of SEQ ID NO: 4 can bind to cells that are at low confluency when grown attached to a surface and can bind better/more extensively to cells that are at high confluency. In some embodiments, peptides having the amino acid sequence of SEQ ID NO: 3 or SEQ ID NO: 5 can bind to cells that are at low confluency when grown attached to a surface and can bind worse/less extensively to cells that are at high confluency. In some embodiments, the cells are prostate cells. In some embodiments, the cells are LNCaP cells.

Generally, the peptides disclosed herein can bind/differentially bind cells. In embodiments, the cells can be from any mammal. In some embodiments, the cells can be human cells. In some embodiments, the cells can be cultured cells. In embodiments, the cells can be primary cells or cell lines. Generally, the peptides disclosed herein can bind/differentially bind to any cell, to any subset of cells, or to any particular cell or cell type. In some examples, the cells to which the peptides can bind are prostate cells. The cells can be prostate cancer cells. In embodiments, the prostate cancer cells can be obtained from the American Type Culture Collection (ATCC). In embodiments, the cells can be LNCaP, DU145, PC3, PC3M, and the like.

### Methods for Identifying Peptides and Assaying Peptide Binding to Cells and Analytes

Herein, methods for identifying peptides that bind cells can use peptides that are associated with a nucleic acid. In some embodiments, the peptide associated with a nucleic acid can be a peptide-nucleic acid hybrid molecule. In some embodiments, the peptide in these associations can be covalently attached to a nucleic acid (see Drygin, Yu F. “Natural covalent complexes of nucleic acids and proteins: some comments on practice and theory on the path from well-known complexes to new ones.” Nucleic acids research 26.21 (1998): 4791-4796). In some embodiments, the peptide associated with a nucleic acid can be a peptide nucleic acid (PNA: see Swenson, Colin S., and Jennifer M. Heemstra. “Peptide nucleic acids harness dual information codes in a single molecule.” Chemical Communications 56.13 (2020): 1926-1935). In some embodiments, peptides can be associated with nucleic acids in the context of a virus (e.g., a bacteriophage or phagemid particle). In some embodiments, the nucleic acid portion of a peptide-nucleic acid association can encode the peptide portion of the association.

Generally, the peptides and associated nucleic acids can be in any configuration as long as the peptide portion of the association can bind to its specific cellular analyte and the nucleic acid portion: i) can be detected and/or quantified as a proxy for peptide binding to the analyte and, optionally, ii) encodes the peptide portion. In embodiments where the nucleic acid portion encodes the peptide portion, the nucleotide sequence of the nucleic acid portion can be determined and used to infer the amino acid sequence of the peptide portion.

In some embodiments, the peptides associated with nucleic acids can themselves be associated with or be part of “particles,” like bacteriophages or phagemid particles that have a nucleic acid genome. In embodiments, the genome of the particles may be a viral/bacteriophage genome, a phagemid, a plasmid, and the like. In embodiments, the genomes may be DNA or RNA. In embodiments, the genomes may be single-stranded or double-stranded.

Bacteriophages that display foreign peptides on their surfaces are known in the art (see Smith, G. P., “Filamentous fusion phage: novel expression vectors that display cloned antigens on the virion surface.” Science, 228.4705 (1985): 1315-7). In some embodiments, a nucleic acid sequence encoding a peptide is part of viral genome sequences that encode coat/capsid proteins of the virus. Expression of such a nucleic acid can result in a viral coat/peptide fusion protein, where the peptide is displayed on the surface of the virus or bacteriophage and is available for binding to a specific analyte. Libraries of bacteriophages that contain, in some embodiments, up to 10⁹ or more unique random peptides are known. In some embodiments, the bacteriophages may be filamentous bacteriophages.

Bacteriophages in these libraries that display peptides, and that can bind to cells/cell analytes through their displayed peptides can be identified using strategies as described in nonlimiting Example 1 of this application. In some embodiments, bacteriophages in a library of bacteriophages that encode different peptides and display the peptides on the surface of individual bacteriophages can be contacted with cells under conditions where the displayed peptides can bind to analytes on the cells. In some embodiments, the cells can have a desired first condition or state. For example, the cells may have a high passage number or low passage number. The cells may be more confluent or less confluent. Bacteriophages that bind to cells can be isolated (bacteriophages that do not bind to cells can be washed away) and individual phages can be tested for those that saturably bind the cells (i.e., at some point, as more bacteriophages are added to the cells, the cells do not bind more of the bacteriophages).

Once bacteriophages that can bind/saturably bind to cells/cell analytes are identified, sequence of the genome of the bacteriophage can identify the nucleotide sequence in the genome that encodes the peptide. The amino acid sequence of the peptide can be inferred from the nucleotide sequence. Data from these types of studies are described in nonlimiting Example 2 of this application.

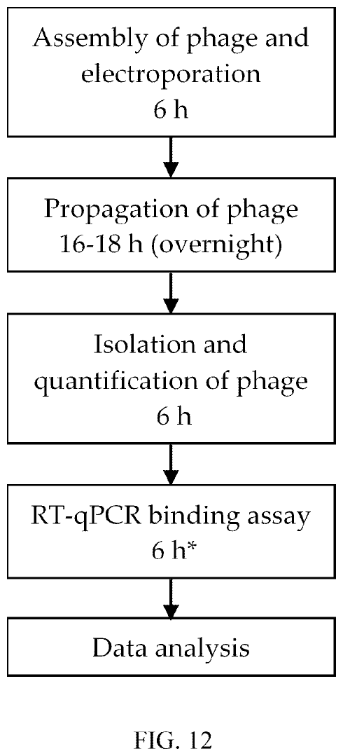

Once a nucleotide sequence that encodes a peptide is obtained from a bacteriophage genome, for example, the bacteriophage from which the nucleotide sequence is obtained can be reconstructed as described in nonlimiting Example 3 and shown in FIG. 12. In embodiments, the nucleotide sequence of the peptide can be used to synthesize an oligonucleotide that encodes the peptide, and the oligonucleotide can be cloned/constructed into a bacteriophage/phagemid/plasmid genome. This genome can be introduced into a bacterial cell (e.g., using transformation, electroporation, and the like) so that the bacteria can replicate and reproduce bacteriophages or phagemid particles.

As described in nonlimiting Example 3, this reconstruction process can be used in high throughput propagation and screening of bacteriophage clones. These methods can speed up propagation and screening of bacteriophage clones by identifying bacteriophages using the nucleotide sequences of their genomes and then reconstructing the bacteriophages and minimizing the use of isolating and propagating individual bacteriophages on bacterial cells.

Bacteriophages that saturably bind to cells under the first condition, as described above, can be tested for their ability to bind to cells under a second condition. For example, if the first condition is low cell passage number, the second condition can be high cell passage number. If the first condition is high cell passage number, the second condition can be low cell passage number. If the first condition is high cell confluency, the second condition can be low cell confluency. If the first condition is low cell confluency, the second condition can be high cell confluency.

Peptides that differentially bind to cells and their analytes under many different conditions can be detected. Generally, as long as one can detect peptide/bacteriophage binding under a first condition and detect a change in binding (e.g., higher or lower binding) under a second condition, peptides that differentially bind can be detected. In some embodiments, one may screen for peptides/bacteriophages that do not bind or bind at a low level under the first condition and bind or bind at a higher level under the second condition. Example first and second conditions, for example, can include cell age/cell passage number (e.g., low or high), cell confluency/cell density (e.g., low or high), identity of a cell (e.g., peptides that bind to a specific cell line, to cells from a specific tissue, from a specific species, and the like, but differentially bind to other cells), cell growth rate, cell cycle state (e.g., G1, S, G2/M), media composition (e.g., good or bad for cell viability, growth, metabolism and the like), oxygen consumption, extracellular acidification rate, production of specific substances by cells (e.g., proteins/recombinant proteins, toxic substances, and the like). Differential binding of the peptides, and the corresponding cellular analytes to which the peptides differentially bind, under these or other conditions or states, can be correlated with the presence or absence of properties or combinations of properties (e.g., cell phenotypes) displayed by the cells.

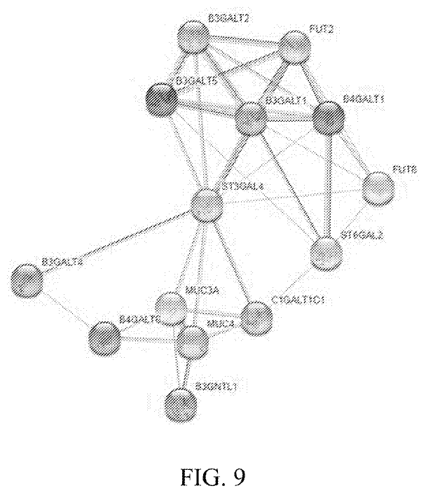

In some embodiments, genes involved in synthesis or regulation of an analyte to which a peptide binds can be identified (see nonlimiting Examples 1, 2 and 4). For example, a CRISPR knockout library in cells to which a peptide binds can be constructed. The library can be screened for cells containing a knockout that eliminates or decreases binding of the peptide under conditions in which the peptide should bind the cells. Identification of the gene or genes associated with the location that has been knocked out in the cell can identify these genes and/or pathways. Data from these types of studies are described in nonlimiting Example 2 and shown in FIGS. 9-11.

Once peptides that can bind/differentially bind to cells/analytes are identified, the peptides in association with a nucleic acid can be used to measure and/or quantify peptide binding to the cells and/or analytes, and/or to determine a condition, state or phenotype of a cell. The peptides in association with a nucleic acid can be, for example, peptide-nucleic acid hybrids, peptide nucleic acids (PNAs), viruses, and the like, as described at the beginning of this section of this application. Detection and/or quantification of peptide binding to cells and their analytes can be used to determine whether cells, for example, possess certain conditions or states that can be used to define cell phenotypes. Example studies of this type are found in nonlimiting Example 2 of this application.

In some embodiments, a peptide associated with a nucleic acid is contacted with a cell analyte and/or cell containing the analyte under conditions in which the peptide can bind to the analyte and/or cell. Binding of the peptide to the analyte and/or cell is detected/measured/quantified using the nucleic acid molecule associated with the bound peptide. In some embodiments, the nucleic acid associated with the bound peptide is detected using polymerase chain reaction (PCR). In embodiments, the PCR can be quantitative PCR (qPCR).

In some embodiments, the peptide associated with a nucleic acid is a virus that displays the peptide on the virus surface. In some embodiments, the peptide associated with a nucleic acid can be a bacteriophage or phagemid particle that contains a genome. In embodiments, the virus, bacteriophage, or phagemid particle is contacted with cells under conditions in which the virus, bacteriophage or phagemid particle can bind specifically to an analyte on the cells. Virus not binding to the analyte on the cells can be washed away. Virus binding to the analyte on the cells, through the displayed peptide, can be quantified by detection of the nucleic acid genome of virus bound to the analytes. In some embodiments, the detection of the viral nucleic acid genome can use PCR. In embodiments, the PCR can be qPCR.

As described earlier in this section, genes involved in synthesis or regulation of analytes bound by peptides can be identified, in some examples using CRISPR knockout libraries. In some examples, quantification of peptide binding to cells, as described above, can be used in combination with detection/quantification of RNA (e.g., mRNA) encoded by the genes involved in synthesis or regulation of analytes bound by the peptides. In embodiments, comparison of amounts of a cellular analyte, though peptide binding as described above, with amounts of mRNA that encode the peptide or encode regulators of the peptide, can provide additional insights into cellular phenotype. In some embodiments, mRNA amounts can be quantified using PCR. In embodiments, mRNA amounts can be quantified using real-time quantitative reverse transcription PCR (qRT-PCR).

In some embodiments, amounts of analyte (as determined by peptide binding) and/or amounts of mRNA can be used in combination with analysis of the genome of cells. Determination of this DNA parameter can provide additional insights into cellular phenotype. In some embodiments, cell genomes can be probed or analyzed using short tandem repeat (STR) profiling.

### Applications of Peptides

The peptides described herein can have a variety of uses. In some embodiments, the peptides can be used alone. In some embodiments, the peptides described herein can be used in the context of the peptide associated with a nucleic acid (e.g., bacteriophage). In some embodiments, the peptides can be used to identify, measure and/or quantify peptide binding to cells and/or analytes. In some embodiments, the peptides can be used to target (e.g., bind to) molecules/biomarkers of cells in imaging, therapeutic, diagnostic, and other methods.

In some embodiments, the peptides described herein, and/or molecules associated with the peptides (e.g., a nucleic acid, a bacteriophage) can be labeled. Generally, the labeling can be any type of labeling used in biological or biochemical applications. In some embodiments, the labeling can include fluorescent labels, chemiluminescent labels, enzymatic labels, chemical labels, labeling using a peptide tag, biotin/digoxigenin labeling, radionuclide labeling, and the like.

In some embodiments, the peptides can be used in imaging. The peptides or molecules associated with the peptides (e.g., molecules of a bacteriophage coat) can be labeled. In some embodiments, the peptides can be labeled in such a way that the label and associated peptides can be detected by various types of imaging. Example types of imaging can be positron emission tomography (PET), single-photon emission computed tomography (SPECT), magnetic resonance imaging (MRI), photoluminescence imagine (PL), and others. In an example embodiment, a peptide that is specific for binding to a cancer cell or a type of cancer cell can be labeled, contacted with cells from a patient or administered to a patient, and detection of the label can be indicative of cancer in the patient.

In some embodiments, the peptides can be used in a therapeutic molecule. In some embodiments, the peptides can be conjugated to a drug, radionuclide, or other therapeutic to target the therapeutic to a particular cell. In some embodiments, the peptides can be conjugated to or associated with a nanoparticle or liposome, for example, to target the nanoparticle/liposome or their contents to a particular cell. In some embodiments, the peptides can be attached to, for example, a peptide that has biological activity to target the peptide to a particular cell. Administration of any of these peptide conjugates can target the attached drug, radionuclide, and the like, to a desired location in the body after administration to a patient. In some embodiments, the peptides that are specific for binding to a cancer cell or a type of cancer cell can be used to target these and other types of therapeutics to the cancer in a patient.

In some embodiments, the peptides can be used in various diagnostic assays. In some embodiments, the peptides or molecules associated with the peptides (e.g., molecules of a bacteriophage coat) can be labeled in such a way that they can be detected by various assay methods. Example types of assay methods can be enzyme-linked immunoassay (ELISA), bead-based luminescent amplification methods (e.g., AlphaLISA), SPR assay (e.g., Biacore), quantitative polymerase chain reaction (qPCR), microscopy, and the like. In some embodiments, the peptides that have been labeled so they can be detected in an assay, can be contacted with a patient sample. Detection of the labeled peptide or associated molecule can be diagnostic for a condition, disease, and the like. In some embodiments, peptides that are specific for binding to a cancer or a type of cancer cell can be used to identify the cancer in a patient sample using these various assays.

In some embodiments, peptides as described herein can be used as replacements for antibodies. In some embodiments, the peptides can be used as replacements for anti-idiotypic antibodies.

In some embodiments, binding of peptides as described herein, to an analyte, may indicate that the analyte to which it binds is bioactive (e.g., the peptide cannot bind to non-bioactive analytes). In some embodiments, the peptides can be used in cosmetology.

### Kits

Kits containing any of the reagents (e.g., peptides, nucleic acids, bacteriophages, and the like) and/or for use in any of the methods disclosed herein are also contemplated in this application.

In some embodiments, the kits can contain any of the peptides, bacteriophages, phagemid particles, genomes of bacteriophages or phagemid particle, and the like, disclosed herein. In some embodiments, the peptides, bacteriophages, phagemid particles, genomes of bacteriophages or phagemid particle, and the like, can be labeled. In some embodiments, the label can be a fluorescent tag, biotin tag, or other affinity tag (e.g., for detection by a plate reader, flow cytometry, and the like) for use in a kit.

In some embodiments, the peptides, bacteriophages, phagemid particles, genomes of bacteriophages or phagemid particles, and the like, labeled or unlabeled, can be part of a kit used for flow cytometry detection of cells or cell sorting to identify and/or isolate subpopulations of cells. In some embodiments, the kits can be used for microscopy, histochemistry, and the like. In some embodiments, the peptides, bacteriophages, phagemid particles, genomes of bacteriophages or phagemid particle, and the like in a kit can be used in place of antibodies, for example, in typical laboratory applications.

In some embodiments, the kits can contain reagents (e.g., templates and/or PCR primers) for detecting/quantifying analytes, mRNA or genome sequences, as described herein.

In some embodiments, the kits can contain instructions for using the reagents and/or for performing the methods.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Specific Aims: Experiments are proposed to develop a kit/service able to both identify and characterize cell lines by dually targeting and assessing both mRNA transcripts and cell surface biomarkers as markers of phenotype. This kit/service will also work with Short Tandem Repeat analysis to enable scientists to probe three tiers of cell line biology; DNA sequence, RNA transcription, and biomarker expression. With these methods, it is possible to quantify non-traditional phenotypic markers in a sensitive, quantitative, easy to use, and cost-effective manner: using novel peptide and DNA primer sequences. In short, bacteriophage (o) expressing multiple copies of a targeting peptide will be used to bind a cell surface biomarker. The ssDNA genome of the o, with the unique foreign peptide genetic sequence, can be used to translate protein/carbohydrate/lipid biomarkers into a PCR based quantifiable signal. Quantification of bound ø via qPCR can be paired to qRT-PCR analysis of correlating mRNA transcripts. The final kit will include many unique targeting ø clones for incubation with any available cell line. After free ø are washed away and total nucleic acid isolated, kit provided primers specific for mRNA transcripts and ø clones can be used for qRT-PCR/qPCR quantification. The resulting Ct values can then be normalized and compared to a database containing validated levels of both mRNA and biomarker expression. Analysis of a matrix of biomarkers/ø clones/mRNA transcripts allows for sensitive detection of both the presence/absence of biomarkers. The ability to directly compare changing mRNA and cell surface biomarker levels over the course of time can provide useful information about altered biochemical pathways. In this way, we can use a single common protocol to probe an array of different cell types/lines. Experiments will be reported along with the expression levels of the mRNA/biomarkers within that cell line's matrix. This information will identify aged or inappropriately cultured cells, evaluate published data, and aid in the production of reproducible results.

In some embodiments, kits for the identification and characterization of ATCC prostate cell lines will be developed. The kit can identify the type of cell line and detect the presence of cells that are “drifting” or “ageing” due to improper conditions or over culturing. In embodiments, this concept can be utilized for many different cell types. In some embodiments, a panel of ø clones with identified biomarkers for LNCaP cells is disclosed. In some embodiments, a database of phenotype markers is disclosed for all prostate cell lines available from ATCC. In some embodiments. RNAi is used to correlate mRNA variants and biomarker expression levels. While not wishing to be bound by theory, the combined data from all ATCC human prostate cell lines including identification of many biomarkers, their appropriate expression levels, and the functional relationships between the genes will aid in the fine tuning and simplification of the final panel of ø clones for the prostate cell line kit. The disclosed methods can verify the phenotypic profile of the tested cells, verify optimum culturing conditions and/or age of the tested cells, and discern “drifting” within the cells.

Parallel ø display selections against low and high passage number LNCaP cells. In some embodiments, parallel ø display selections against low/high passage number LNCaP prostate carcinoma cells were performed. Two selection techniques, traditional and depletion, were used for both high and low abundance biomarkers. The genetic sequence of the peptides were PCR amplified and sequenced using Illumina next-generation sequencer. Finally, the resulting sequences were trimmed, translated, and sorted. Positive selection sequences were cleared of negative control sequences and known “target unrelated peptide” clones. Finally, sequences were weighted by the number of identical clones and via motif identifying software.

Screening of selected ø clones. Ø clones were reconstructed and the clonal populations biotinylated. Biotinylated ø were used in a cell-based ELISA for 4 rounds of screening. The 1st round of screening identified clones with saturable binding and the apparent affinity (Bmax and KD) for prostate carcinoma and normal lines. Specificity was tested by cell binding to breast carcinoma, colon cancer, and HEK293 cells. The 2nd round of screening used the same cells and clones in cell binding assays once a month for 4 months, plus a 7-month old LNCAP was used to observe binding correlating with passage number. Rounds 3 and 4 screening repeated the protocol described above though using cells grown in media of a different composition (round 3) or cells grown at different confluence (round 4). In this way, we identified ø clones/biomarkers sensitive to many of the common variabilities found between laboratories.

Utilize CRISPR knockout library to identify gene(s) required for biomarker expression. The ø clones of interest from above were utilized in a loss of binding screen on LNCaP cells transfected using a genome wide CRISPR knockout library. A Beckman Coulter MoFlo XDP was used for cell sorting flow cytometry to isolate LNCaP cells with little to no ø binding. Transfected gRNA of the isolated cells was identified using next generation sequencing and web-based software was used to determine the functional relationships amongst the identified genes.

Significance: The persistent existence of contaminated/misidentified cell lines (CMCL) within the scientific community is a significant problem [1]. The continued use of CMCL is affecting scientific research and thus impacting the development of medically important drugs [2-5]. HeLa contamination was first noted in the literature in the 1960's [6, 7]. CMCL have continuously been utilized through the decades with a large percentage being either contaminated with HeLa or mislabeled [4, 6, 8-10]. Recently published papers reporting large scale use of CMCL predict that 20%-30% of all cell lines in use may be CMCL [1, 7, 10, 13]. The presence of “false” cell lines has been attributed to lack of awareness, documentation, and access to tools/equipment needed for verification.

Additional problems of over-culturing are beginning to be addressed within literature. Culturing cell lines for too long with various unintentional selective pressures results in drift [14-16]. This in turn generates a myriad of cell lines with the same name but unpredictable, reduced, and/or altered biology [5, 17]. These divergent cell lines exhibit different gene expression leading to different morphology, development, and key functions [16, 18, 19]. Thus, reproducible results between laboratories are negatively affected. Additionally, generated data is no longer representative of the original source material [5]. For example, high passage number human prostate carcinoma cells. LNCaP, respond differently to androgens and retinoids when compared to low passage number cells [20, 21]. It is well documented that passage number, media, and seeding density all affect morphology, proliferation rate, cell density, glucose transporter expression, and brush border enzyme activities of human intestinal Caco-2 cells [17, 22-26]. These reports highlight the sensitivity of mammalian cells to their environment.

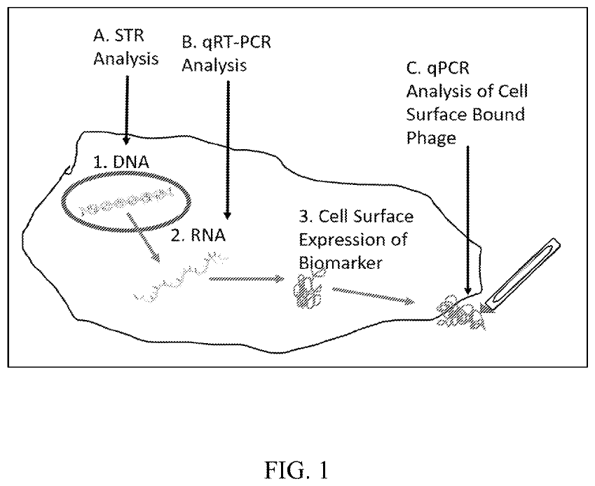

These combined studies underscore multiple pitfalls (CMCL, drifting phenotype, and/or divergent evolution) currently preventing the goal of improved reproducibility. Importantly, there is currently no way to methodically verify the phenotypic status of cell lines within the common laboratory, thus, the scientific community continues to build upon research performed with CMCL and/or cells with changed phenotype [1-3, 5, 10]. Proposals to overcome this problem include improved documentation and reporting of spurious results within the literature [13, 14, 27]. A feasible option is to develop a cheap and easy to use kit or service to monitor actively growing cultures for drifting phenotype. Results published from all cell lines should have short tandem repeat (STR) analytics and a description of the phenotype (both mRNA and biomarker) profiles of the cell line at the time of the experiment (FIG. 1). This in turn would provide researchers with valuable information for comparison of data between research groups and potentially help explain the differences in results.

Currently, STR analysis can genetically identify cell lines and has been validated by American National Standards Institute and the American Type Culture Collection (ATCC) resulting in a documentary standard (ASN-0002) [11, 12, 28]. However, STR analysis is unable to identify spontaneous mutations outside of the amplicon region or identify cell lines that are genetically identical but phenotypically different. Our approach is development of a dual targeting tissue type specific kit able to describe the phenotype of a cell line at the time of the experiment. This kit can be provided to the end user directly or may be offered as a service. This kit can 1) verify tissue type, 2) verify cell line. 3) verify lack of contamination by other cell types, and 4) be able to discern “drifting” cell lines. Here “drift” is defined as biochemical changes resulting from epigenetic, transcriptional and/or translational changes, or changes to post-translational modification or processing. This kit will target and quantify both mRNA and cell surface biomarker levels to be able to truly monitor phenotype.

Innovation: While there are many proposals for the quality control and identification of cell lines, most of them focus on either DNA/RNA/protein expression profiling or rely upon characterization of a single biochemical pathway (e.g., receptor signaling) [15, 20, 21, 27, 28]. These approaches are unable to encompass a broad biochemical profile of a cell line, and thus, are unable to truly monitor a cell line for phenotypic drift. Partially because of the inability of these techniques to monitor expression of lipid and/or carbohydrate antigens. Herein, however, disclosed is a unique protocol able to truly evaluate the phenotypic status of a cell line by quantifying both mRNA and all forms of associated cell surface biomarkers. Filamentous bacteriophages (ø) containing a single stranded DNA (ssDNA) genome genetically modified to express a unique foreign targeting peptide [29] are used. This targeting peptide can possess specificity and affinity for any type of cell surface target [30]. This in turn allows us to utilize the ssDNA of cell surface bound ø as a nucleic tag able to translate protein/lipid/carbohydrate expression into a polymerase chain reaction (PCR) based quantifiable signal (FIG. 1). Additionally, this PCR assessment of ø ssDNA is easily coupled to the analysis of cellular mRNA sequence. This is the basis of a kit that would contain multiple unique ø clones each expressing a different targeting peptide, thus allowing for quick and efficient investigation of multiple biomarkers along with associated mRNA transcripts. Simultaneous probing of multiple mRNA/biomarker pairs into a single PCR based system can provide direct evidence of tissue type, cell line identity, lack of common contaminating cell lines, as well as be able to provide initial evidence of phenotypic drift. Directly comparing changing mRNA levels to changing cell surface biomarker levels within the same cell line over the course of time will provide additional information about altered biochemical pathways. Use of this proposed product in conjugation with STR analysis enables probing three tiers of cell biology. Finally, the combination of two distinct blinded combinatorial techniques, ø display (PD) and clustered regularly interspaced short palindromic repeats (CRISPR) knockout libraries, is disclosed. To date, the identification of a single PD selected clone's binding partner/biomarker has required intense scrutiny and labor. Utilization of a CRISPR whole genome knockout library enables the identification of a family of functionally related genes required for o/biomarker binding phenotypically relevant mRNA transcripts directly related to the selected clones/biomarkers. The disclosed techniques probe more forms of biologically relevant biomarkers (lipids and carbohydrates) in a way that the “-omics” technology is not currently. Unfortunately, to date, there are no forms of “-omics” able to probe network signaling and/or noncanonical biomarker status. The disclosed kit offered to individual laboratories will be low-throughput, however, the offered service can easily be scaled up via robotics and other high-throughput technologies to include increasing numbers and complexities of biomarker surveillance.

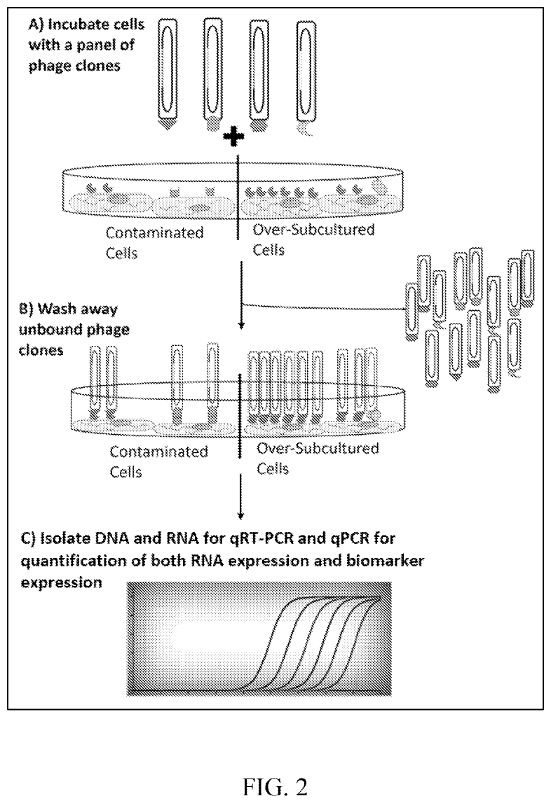

This disclosure provides a panel of multiple ø clones, each specific for a different biomarker, will be utilized to simultaneously probe the status of multiple cell surface biomarkers (FIG. 2). Each paired ø clone/biomarker is characterized and validated for a known expression level within multiple cell lines and specific tissue type (LNCaP vs DU145 vs PC3, etc). Furthermore, the levels of expressed mRNA transcript(s) associated with biomarker expression are also characterized and validated. A final matrix (or scorecard) of biomarkers/ø clones/mRNA transcripts allows for the sensitive detection of the presence/absence of important biomarkers. Utilization of a matrix database (or scorecard) system then enables the identification of drifting cells (ie. Changes in biomarker/mRNA levels) due to prolonged culturing, inappropriate media composition, confluency, and identification of contaminating cells. In this way, we can specifically probe for numerous biomarkers in an array of different cell lines using a single common panel of ø clones. Importantly, this kit will employ common techniques in an innovative way. Thus, the proposed kit meets the FOA requests of; “reliable, rapid, cost effective, and easy to use”, “facilitate the type of frequent, small-scale use prevalent in individual laboratories”, and provide “method for distinguishing between cell lines based on phenotype [and] signaling network activities.”

There exists a need for a kit/service able to describe mRNA transcript/biomarker status within a cell line. This is due specifically to the unfortunate fact that many researchers may be slow to relinquish old frozen stocks, stocks with incomplete historical data, and/or unique cell lines derived from questionable stock. Thus, a common, cheap, and easy-to-use kit (or service) with a long shelf-life, used to determine/verify biomarker expression levels may help change the scientific culture. If a researcher is not amenable to removing questionable cells from their laboratory, they may report the biomarker matrix along with experimental results. This added information can aid the scientific community in evaluating data. Immune-polymerase chain reaction (IPCR), a PCR template conjugated to an antibody, has been utilized within various protocols for the detection of ultra-low concentrations of antigen(s) [31, 32]. An IPCR ELISA possesses a linear dynamic range of ˜6 orders of magnitude with an associated gain of sensitivity of about 1,000-10,000-fold greater than a traditional ELISA [32]. Unfortunately, production of antibodies and coupling to oligoes are time consuming, expensive, and the antibody displays shortened shelf life. Instead, use of ø has been successfully demonstrated in ELISA format [33]. The ssDNA is protected from the environment by coat proteins and is well known to be very stable within a range of temperatures, pH's, and other harsh conditions [30, 34, 35]. Importantly, production and purification of ø is extremely cheap [30, 34, 35]. These factors enable a novel, stable, and cheap kit able to target nontraditional biomarkers.

PD technology has been repeatedly used throughout the past 30 years to identify cell, tissue, and organ specific peptides [35]. The strength of PD selection lays in the “genotype-phenotype link”, which enables blinded combinatorial selection of peptides that can bind to any part of the cell surface [35]. Two different basic selection types, traditional and depletion, are often used [35-37]. In the traditional PD technique, the ø library is incubated with cells and bound ø are eluted. This process is repeated multiple times. The remaining peptide sequences are amplified and then compete for binding sites within subsequent rounds of selection. However, the competition for binding sites upon whole cells is not very robust and the resulting identified ø clones usually bind high abundance biomarkers. To the contrary, in a depletion PD protocol, the ø library is incubated with cells, and unbound ø are collected. In this way, non-specific and high abundance binders are removed. This depletion aids in identifying peptides specific for biomarkers displayed at low levels. Parallel PD selections using both protocols will significantly increase the likelihood of selecting tissue/cell type specific and phenotype specific peptides.

Development of CRISPR/Cas9 gene editing technology has proven itself to be a powerful method [38, 39]. Genome wide Traditionally, targeted gene editing for the study of biology focused on individual genes [40]. However, laboratories have recently developed CRISPR knockout libraries are commercially available [41, 42]. Consequently, large scale screening of cells based on loss of function combined with high-throughput analysis has unlocked a potent tool for target discovery. Use of a genome wide knockout library for the identification of biomarkers targeted by peptide-displaying ø is a novel technique not yet reported in the literature, and will allow for an unprecedented level of blinded, combinatorial investigation into all forms of cell surface biomarkers (protein, lipid, carbohydrate, etc). With this technique we will be able to identify groups of functionally related genes. And thus, determining whether the targeted biomarker is a product of the canonical protein expression pathway requires the functional pathways of carboxylation/lipidation, or other forms of biomarker regulation.

Objectives: In some embodiments, the proposed kit will include a panel of ø clones for validation of cell lines. In some embodiments, the cell line can be any prostate cell line offered by ATCC. The ø panel can be incubated with an actively growing human prostate cell line available from ATCC. Free, unbound ø will be washed away and total cell and ø nucleic acids, isolated and purified. Next, quantitative PCR/quantitative reverse transcriptase-PCR (qPCR/qRT-PCR) protocols will be performed to quantitate i) mRNA transcripts and ii) associated biomarker targeting ø clones. Finally, the resulting Ct values from qPCR and qRT-PCR will be normalized for comparison to a matrix of validated expression levels of each mRNA/ø clone/biomarker. The information obtained from the kit/service can i) (in) validate the tested cell line, and/or ii) provide evidence of drift.

In some embodiments, the human prostate carcinoma cell line, LNCaP can be used. Included is identification of a panel of ø clones, initial characterization of their cell binding specificity, and identification of genes necessary for the expression of the biomarker. The ø clone panel includes individual ø clones that specifically identify i) prostate tissue biomarkers, ii) LNCaP biomarkers, iii) low vs high passage number biomarkers, iv) biomarkers sensitive to media composition, and v) biomarkers sensitive to cell density. Parallel PD selections utilizing traditional and depletion PD protocols are used. The resulting ø clones of interest were subjected to four rounds of screening. Once ø clones of interest were identified and cell binding characterized, a CRISPR knockout library was utilized to probe for genes required for expression of each biomarker. This allowed for identification of each biomarker and characterization/validation of biomarkers. PD selections, screening, and CRISPR mediated identification of biomarkers will experimentally confirm “proof of concept” and feasibility of a commercial kit/service.

In some embodiments, additional PD selections, screening, and CRISPR identification of biomarkers for the other human prostate cell lines available from ATCC can be performed. This will greatly increase the complexity of the panel of mRNA/o/biomarkers requiring more high-throughput automated technologies, such as robotics. RNAi can be used to outline relationships between mRNA variants and biomarker, as well as define the relation of the gene to the biomarker. Importantly, functional relationships between the genes can be investigated and biological relevance of the biomarkers probed. Validation of each o/target can be performed by acquiring Ct values from the qPCR/qRTPCR protocol from across this large panel of similar and dissimilar cell lines. While not wishing to be bound by theory, individual ø clones may not have 100% specificity for a specific cell line. Expression of at least some identified biomarkers have a defined range that may differ between cell lines/cell types. Biomarker expressed on cell lines derived from similar source material (ie. DU145, PC3, PC3M, etc.) may be similar, but each cell line can possess its own unique combination of biomarkers and expression levels. In some embodiments, a database (matrix) containing the gene expression profiles for each o/biomarker/mRNA transcript is developed. This matrix can be used to determine a final panel of ø clones that are able to specifically identify and monitor, in some embodiments, any prostate cell line offered by ATCC. In some embodiments, kits may include multi-fluorescent flow cytometry/cell sorting for simultaneous cell characterization and elimination of contaminating cells. While not wishing to be bound by theory, our ø panels can be useful for detection and correction of low levels of contaminating cells (10% or less). In some embodiments, kits will include ø panels for mouse, rat, human, and other specific cell types to address issues of cross species contamination. In some embodiments, kits can include: breast, kidney, lung, liver, ovary, etc. tissue culture cell lines.

Data: The HER-2 receptor is a receptor tyrosine kinase (RTK), a member of the epidermal growth factor receptor family, is known to be expressed on LNCaP cells, and is strongly implicated in the development and progression of prostate cancer [43]. A HER-2 targeting ø clone. KCCYSL [44-46] has been developed. KCCYSL has been successfully utilized as both a tumor targeting ø clone, as well as a tumor specific peptide [45-47]. This ø clone will be a positive control (as a known biomarker and gene) within this study. Comparatively, the Thomsen-Friedenreich (TF) antigen is a carbohydrate tumor antigen [48]. It is a Core-1 (Galβ1-3GalNAcα-1Thr/Ser) intermediate structure of O-glycans [49]. Many groups have independently verified the tumor specificity of TF antigen, and most show that TF antigen is a result of truncated glycosylation [48-51]. An ø clone, p30-1, specific for TF antigen [52-56] has been identified, maturated and characterized. p30-1 will be utilized, within the disclosed studies, as a positive control (as a known biomarker, but unknown gene(s)). For example, we showed the anti-TF ø, p30-1, binding TF positive human breast carcinoma and SKOV-3 tumors in vivo. While the KCCYSL and p30-1 ø clones were identified from PD selections against purified antigen, the previously selected ø clones, H5 and G1, were PD selected against whole PC-3 human prostate carcinoma cells [46, 47, 57]. These results highlight the ability of this type of protocol to isolate peptides/biomarkers that range from a propensity of carcinoma cell binding to binding only prostate carcinoma cell lines. Importantly, the levels of ø binding (fluorescence intensity) are unique for each cell type.

Parallel PD selections against low and high passage number LNCAP cells. All cell lines utilized for proposed experiments were obtained from ATCC and maintained as described by ATCC. The 1st thaw and expansion included freezing back multiple vials of “first passage” cells. ATCC's STR Profiling Cell Authentication Service can be used each time a vial is thawed. The 1st flask of thawed LNCaP cells was split into two “lines”. The 1st line can be continuously passaged for 4-7 months and then submitted to the described PD selection. The 2nd line was immediately expanded for use in round 1 of the two distinct PD selection protocols (traditional and depletion). To keep all conditions consistent, the two PD protocols were performed within the same 6-well plate. Two wells were used for each of the following conditions a) growth media without cells (negative control), b) LNCaP cells (80% confluency) for traditional PD selection, and c) LNCaP cells (80% confluency) for depletion PD selection. And to better enable a service, the cells were rinsed and then fixed with 10% buffered formalin (BF) prior to ø display selection. In this way, researchers looking for a screening service can send plates of fixed cells to us for testing. A naïve fUSE5 15-mer random peptide PD library was incubated within each of the six wells. For the control and traditional selection, fixed cells were washed, and bound ø eluted. For the depletion selection, unbound ø from the supernatant was collected. At this point, an aliquot of ø was removed for Illumina next generation sequencing (NGS) to monitor the selection process. Ø from the duplicate selections were pooled, amplified, and used in subsequent rounds generally 4 rounds). For the traditional and depletion protocol, the last round of selection collected ø that bound to the cells. Eluted ø DNA from all wells was immediately isolated and Illumina adapters were added to the DNA sequence for each peptide using PCR. All six variables (negative control, traditional, and depletion selection ø from both new and 4-month-old LNCaP cells) were submitted for NGS. Subsequent DNA sequence data was submitted for trimming, translation, and sorting. At this time sequences identified within both experimental and negative control samples set were eliminated. In this way, we control ø clones displaying peptides that bind to plastic, plastic bound serum proteins, or other non-specific backgrounds. Sequences were weighted by the number of clones displaying the same peptide sequence and via motif identifying software. Finally, sequences of interest were compared to databases of “target unrelated peptide” [58, 59].

In some embodiments, 4 rounds of selection might be too stringent in the depletion selection protocol, thus, ø clones identified in earlier rounds of selection may be used.

Screening of selected ø clones. Ø clones of interest were reconstructed. fUSE5 vector (GenBank accession AF218364) was digested with SfiI, hybridized synthesized oligos ligated into the vector, electroporated into E. coli, sequence verified, and ø purified. Ø clones p30-1 (TF targeting), KCCYSL (SEQ ID NO: 9) (HER-2 targeting), or no foreign peptide (neg) were utilized throughout the screening as positive and negative controls. Each ø was biotinylated using N-hydroxysuccinimide (NHS) ester chemistry for the covalent linkage of NHS-PEO4-biotin to the terminal amine of ø coat protein VIII [60]. These biotinylated ø were then used within round I screening via modified ELISA protocol [61]. LNCaP cells were grown within 96-well tissue culture treated plates, rinsed, and fixed with 10% BF. Serial dilutions of biotinylated ø clones were incubated in the presence and absence of LNCaP cells, unbound ø washed away, and bound ø detected via streptavidin conjugated to horseradish peroxidase. Ø found to be plastic binders were eliminated and the apparent affinity (Bmax and KD) of each ø clone for LNCaP cells determined. Ø clones with no saturation of binding were eliminated from the panel. Next, the screening was expanded to include human prostate carcinoma cell lines PC3, DU145, and NCI-H660 and normal prostate cell lines, RWPE-1, RWPE-2, and PWR-1E. In some embodiments, specificity of clones for prostate cells can be tested by expanding the experiments to include breast carcinoma MCF-7 and MDA-MB-231, colon cancer LS174T, and HEK293 cells. Apparent affinity of ø clones for each cell line can be determined and entered into a preliminary database. From these data, only ø clones with statistically significant differences in binding for the various variables (e.g., carcinoma vs normal. LNCaP vs other prostate cell lines, etc) were utilized within the CRISPR knockout library experiments.

Round 2 screening probed for ø clones that bind preferentially to either low or high passage number human prostate cells. All of the same ø clones again were utilized within the above-described cell-based ELISA protocol using human prostate carcinoma and normal prostate cell lines. However, within round 2 screening the panel of ø clones were tested on each of the cell lines once a month for 4 months. Additionally, we probed the “first line” of LNCaP previously described, which at this time, was in continuous culture for about 7 months. In this way, we were able to observe increases or decreases of individual ø clone binding correlating with passage number. Rounds 3 and 4 screening repeated the protocol described above though using cells grown in media of a different composition (round 3) or cells grown at 20%, 40%, 60%, 80% and 100% confluence (round 4). In this way, we identified ø clones/biomarkers sensitive to many of the common variabilities found between laboratories.

Utilize CRISPR knockout library to identify gene(s) required for biomarker expression. The ø clones of interest resulting from the 2 rounds of screening previously described were next utilized in a loss of binding screen on LNCaP cells transfected with LentiPool™ Human CRISPR library (ThermoFisher). The commercially available negative and positive control lentivirus particles were used to determine the multiplicity of infection needed for optimal transduction of LNCaP cells. These predetermined variables were then used in a protocol to stably transfect LNCaP cells with Cas9. In short, LNCaP cells grown to a density of ˜50% within a 6 well plate were incubated with Cas9 Lentivirus particles leaving one well with no lentivirus. After which time the media was replaced and the cells allowed to grow for 2 days. Blasticidin selection of transfected cells was performed. The remaining cells were serially diluted, single clones expanded, examined for Cas9 protein expression, and editing efficiency verified. Transduction of LNCaP-Cas9 with the knockout library was performed as described above using Puromycin. In preparation of the loss of binding screen, p30-1, KCCYSL, neg, and each ø of interest was fluorescently labeled using the same chemistry and protocol as described earlier for the ø biotinylation. Ø fluorescently labeled with NHS-AlexaFluor488 were utilized for the development of a cell sorting flow cytometry protocol on a Beckman Coulter MoFlo XDP. After which, the LNCaP-Cas9 knockout library population was utilized in a loss of binding screen. The screen sorted and collected cells with no ø binding from the LNCaP-Cas9 knockout library cells. Finally, gRNA targeted genes within the selected LNCaP-Cas9 knockout library subpopulations can be identified. To this end, genomic DNA can be isolated from each population collected and PCR performed using primers specific for the lentiviral construct. The resulting PCR product was submitted for blunt end ligation of the necessary Illumina adaptor sequences for NGS. DNA sequence was submitted trimming, analysis, and gene identification. Using Feature Annotation Using Nonnegative matrix factorization and GeneMesh web-based software, we were able to determine the functional relationships amongst the identified genes. It is here that the p30-1 and KCCYSL positive control ø were useful. KCCYSL ø targets a known protein product, whose expression follows the canonical DNA-RNA-protein product pathway, while the p30-1 ø targets a carbohydrate antigen whose expression does not follow the straight-forward canonical expression pathways.

At the 12th month, multiple genes were identified for each biomarker/ø clone of the panel resulting in a panel of sensitive and specific ø clones targeting known biomarkers.

### BIBLIOGRAPHY

1. Horbach, S. and W. Halffman, The ghosts of HeLa: How cell line misidentification contaminates the scientific literature. PLOS One, 2017. 12 (10): p. e0186281.
2. Freedman. L. P., Know Thy Cells: Improving Biomedical Research Reproducibility. Sci Transl Med, 2015. 7 (294): p. 294ed7.
3. Freedman, L. P., I. M. Cockburn, and T. S. Simcoe, The Economics of Reproducibility in Preclinical Research. PLOS Biol, 2015. 13 (6): p. e1002165.
4. Vaughan, L., et al., Widespread Use of Misidentified Cell Line KB (HeLa): Incorrect Attribution and Its Impact Revealed through Mining the Scientific Literature. Cancer Res, 2017. 77 (11): p. 2784-2788.
5. Hughes. P., et al., The costs of using unauthenticated, over-passaged cell lines: how much more data do we need? Biotechniques, 2007. 43 (5): p. 575, 577-8, 581-2 passim.
6. Gartler, S. M., Apparent Hela cell contamination of human heteroploid cell lines. Nature, 1968. 217 (5130): p. 750-1.
7. Lavappa, K. S., M. L. Macy, and J. E. Shannon, Examination of ATCC stocks for HeLa marker chromosomes in human cell lines. Nature, 1976. 259 (5540): p. 211-3.
8. Stacey. G. N., Cell culture contamination. Methods Mol Biol. 2011. 731: p. 79-91.
9. Nardone, R. M., Curbing rampant cross-contamination and misidentification of cell lines. Biotechniques. 2008. 45 (3): p. 221-7.
10. Lacroix, M., Persistent use of “false” cell lines. Int J Cancer, 2008. 122 (1): p. 1-4.
11. American Type Culture Collection Standards Development Organization Workgroup, A.S.N., Cell line misidentification: the beginning of the end. Nat Rev Cancer, 2010. 10 (6): p. 441-8.
12. Identity crisis. Nature, 2009. 457 (7232): p. 935-6.
13. Capes-Davis, A., et al., Check your cultures! A list of cross-contaminated or misidentified cell lines. Int J Cancer. 2010. 127 (1): p. 1-8.
14. Freedman, L. P., et al., Reproducibility: changing the policies and culture of cell line authentication. Nat Methods, 2015. 12 (6): p. 493-7.
15. Masters, J. R. and G. N. Stacey, Changing medium and passaging cell lines. Nat Protoc, 2007. 2 (9): p. 2276-84.
16. Mathieson, W., et al., Antimicrobials and in vitro systems: antibiotics and antimycotics alter the proteome of MCF-7 cells in culture. J Cell Biochem, 2011. 112 (8): p. 2170-8.
17. Briske-Anderson, M. J., J. W. Finley, and S. M. Newman, The influence of culture time and passage number on the morphological and physiological development of Caco-2 cells. Proc Soc Exp Biol Med. 1997. 214 (3): p. 248-57.
18. Chang-Liu, C. M. and G. E. Woloschak, Effect of passage number on cellular response to DNA-damaging agents: cell survival and gene expression. Cancer Lett. 1997. 113 (1-2): p. 77-86.
19. Wenger, S. L., et al., Comparison of established cell lines at different passages by karyotype and comparative genomic hybridization. Biosci Rep, 2004. 24 (6): p. 631-9.
20. Esquenet, M., et al., LNCaP prostatic adenocarcinoma cells derived from low and high passage numbers display divergent responses not only to androgens but also to retinoids. J Steroid Biochem Mol Biol, 1997. 62 (5-6): p. 391-9.
21. Langeler, E. G., et al., Effect of culture conditions on androgen sensitivity of the human prostatic cancer cell line LNCaP. Prostate, 1993. 23 (3): p. 213-23.
22. Behrens, I. and T. Kissel, Do cell culture conditions influence the carrier-mediated transport of peptides in Caco-2 cell monolayers? Eur J Pharm Sci, 2003. 19 (5): p. 433-42.
23. Riley, S. A., et al., Active hexose transport across cultured human Caco-2 cells: characterisation and influence of culture conditions. Biochim Biophys Acta, 1991. 1066 (2): p. 175-82.
24. Sambuy, Y., et al., The Caco-2 cell line as a model of the intestinal barrier: influence of cell and culture related factors on Caco-2 cell functional characteristics. Cell Biol Toxicol, 2005. 21 (1): p. 1-26.
25. Yu, H., T. J. Cook, and P. J. Sinko, Evidence for diminished functional expression of intestinal transporters in Caco-2 cell monolayers at high passages. Pharm Res, 1997. 14 (6): p. 757-62.
26. Zucco, F., et al., An inter-laboratory study to evaluate the effects of medium composition on the differentiation and barrier function of Caco-2 cell lines. Altern Lab Anim, 2005. 33 (6): p. 603-18.
27. Geraghty, R. J., et al., Guidelines for the use of cell lines in biomedical research. Br J Cancer, 2014. 111 (6): p. 1021-46.
28. Masters, J. R., et al., Short tandem repeat profiling provides an international reference standard for human cell lines. Proc Natl Acad Sci USA, 2001. 98 (14): p. 8012-7.
29. Smith, G. P., Filamentous fusion phage: novel expression vectors that display cloned antigens on the virion surface. Science, 1985. 228 (4705): p. 1315-7.
30. Smith. G. P. and V. A. Petrenko. Phage Display. Chem Rev. 1997. 97 (2): p. 391-410.
31. Sano, T., C. L. Smith, and C. R. Cantor, Immuno-PCR: very sensitive antigen detection by means of specific antibody-DNA conjugates. Science, 1992. 258 (5079): p. 120-2.
32. Adler, M., R. Wacker, and C. M. Niemeyer, A real-time immuno-PCR assay for routine ultrasensitive quantification of proteins. Biochem Biophys Res Commun, 2003. 308 (2): p. 240-50.
33. Guo, Y. C., et al., Phage display mediated immuno-PCR. Nucleic Acids Res, 2006. 34 (8): p. e62.
34. Smith, G. P. and T. R. Gingrich, Hydroxyapatite chromatography of phage-display virions. Biotechniques, 2005. 39 (6): p. 879-84.
35. Newton, J. and S. L. Deutscher, Phage peptide display. Handb Exp Pharmacol, 2008 (185 Pt 2): p. 145-63.
36. Even-Desrumeaux, K. and P. Chames, Phage display and selections on cells. Methods Mol Biol, 2012. 907: p. 225-35.
37. Fahr, W. and A. Frenzel, Phage Display and Selections on Cells. Methods Mol Biol, 2018. 1701: p. 321-330.
38. Li, Y., et al., The potential application and challenge of powerful CRISPR/Cas9 system in cardiovascular research. Int J Cardiol, 2017. 227: p. 191-193.
39. Men, K., et al., CRISPR/Cas9-mediated correction of human genetic disease. Sci China Life Sci, 2017. 60 (5): p. 447-457.
40. Zhang. Z., et al., CRISPR/Cas9 Genome-Editing System in Human Stem Cells: Current Status and Future Prospects. Mol Ther Nucleic Acids, 2017. 9: p. 230-241.
41. Rauscher. B., et al., GenomeCRISPR—a database for high-throughput CRISPR/Cas9 screens. Nucleic Acids Res, 2017. 45 (D1): p. D679-D686.
42. Kim, H. S., et al., CRISPR/Cas9-mediated gene knockout screens and target identification via wholegenome sequencing uncover host genes required for picomavirus infection. J Biol Chem, 2017. 292 (25): p. 10664-10671.
43. Poovassery, J. S., et al., Antibody targeting of HER2/HER3 signaling overcomes heregulin-induced resistance to PI3K inhibition in prostate cancer. Int J Cancer, 2015. 137 (2): p. 267-77.
44. Karasseva, N. G., et al., Identification and characterization of peptides that bind human ErbB-2 selected from a bacteriophage display library. J Protein Chem. 2002. 21 (4): p. 287-96.
45. Deutscher, S. L., S. D. Figueroa, and S. R. Kumar, In-labeled KCCYSL peptide as an imaging probe for ErbB-2-expressing ovarian carcinomas. J Labelled Comp Radiopharm, 2009. 52 (14): p. 583-590.
46. Newton-Northup, J. R., S. D. Figueroa, and S. L. Deutscher, Streamlined In Vivo Selection and Screening of Human Prostate Carcinoma Avid Phage Particles for Development of Peptide Based In Vivo Tumor Imaging Agents. Combinatorial Chemistry & High Throughput Screening, 2011. 14 (1): p. 9-21.
47. Newton, J. R., et al., In vivo selection of phage for the optical Imaging of PC-3 human prostate carcinoma in mice. Neoplasia, 2006. 8 (9): p. 772-780.
48. Yu, L. G., The oncofetal Thomsen-Friedenreich carbohydrate antigen in cancer progression. Glycoconj J, 2007. 24 (8): p. 411-20.
49. Goletz. S., et al., Thomsen-Friedenreich antigen: the “hidden” tumor antigen. Adv Exp Med Biol, 2003. 535: p. 147-62.
50. Springer, G. F., et al., T antigen, a tumor marker against which breast, lung and pancreas carcinoma patients mount immune responses. Cancer Detect Prev, 1983. 6 (1-2): p. 111-8.
51. Springer, G. F., et al., Further studies on the detection of early lung and breast carcinoma by T antigen. Cancer Detect Prev, 1985. 8 (1-2): p. 95-100.
52. Glinsky, V. V., et al., The role of Thomsen-Friedenreich antigen in adhesion of human breast and prostate cancer cells to the endothelium. Cancer Res, 2001. 61 (12): p. 4851-7.
53. Glinsky, V. V., et al., Effects of Thomsen-Friedenreich antigen-specific peptide P-30 on beta-galactoside mediated homotypic aggregation and adhesion to the endothelium of MDA-MB-435 human breast carcinoma cells. Cancer Res, 2000. 60 (10): p. 2584-8.
54. Kumar, S. R., et al., (64) Cu-labeled peptide for PET of breast carcinomas expressing the Thomsen-Friedenreich carbohydrate antigen. J Nucl Med, 2011. 52 (11): p. 1819-26.
55. Landon, L. A., et al., Combinatorial evolution of high-affinity peptides that bind to the Thomsen-Friedenreich carcinoma antigen. J Protein Chem, 2003. 22 (2): p. 193-204.
56. Peletskaya, E. N., et al., Characterization of peptides that bind the tumor-associated Thomsen-Friedenreich antigen selected from bacteriophage display libraries. J Mol Biol. 1997. 270 (3): p. 374-84.
57. Northup, J. R. and S. L. Deutscher, Cytotoxic Tumor-Targeting Peptides From In Vivo Phage Display. Comb Chem High Throughput Screen, 2016. 19 (5): p. 370-7.
58. Newton-Northup, J. and S. Deutscher, Contending With Target Unrelated Peptides from Phage Display. J Mol Imaging Dynam, 2012. 2: p. e101.
59. Thomas, W. D., M. Golomb, and G. P. Smith, Corruption of phage display libraries by target-unrelated clones: diagnosis and countermeasures. Anal Biochem, 2010. 407 (2): p. 237-40.
60. Jin, X., et al., A generalized kinetic model for amine modification of proteins with application to phage display. Biotechniques, 2009. 46 (3): p. 175-82.
61. Soendergaard, M., J. R. Newton-Northup, and S. L. Deutscher, In vitro high throughput phage display selection of ovarian cancer avid phage clones for near-infrared optical imaging. Comb Chem High Throughput Screen, 2014. 17 (10): p. 859-67.

### Example 2

Additional results from the studies described in nonlimiting Example 1 are described in nonlimiting Example 2.

Parallel phage display selections against low and high passage number LNCaP cells. Perform parallel phage display selections against low/high passage number LNCaP prostate carcinoma cells were performed. Two selection techniques, traditional and depletion, were used for both high and low abundance biomarkers.

Results: All phage display selections against LNCaP (experimental) and HEK293 (control) cells lines were successfully completed.

Details: Phage display selections against complex selectors (i.e., Cells) can result in an array of peptide sequences with little to no sequence similarity and/or identifiable motifs, leaving the researcher with the potential to screen thousands of selected peptide sequences. Thus, parallel phage display selections were performed, each with multiple variables, in an effort to aid in the identification of lead peptide sequences. Two parallel phage display selections were performed against both LNCaP and HEK293 (control) cells. The first selection was performed using low passage number cells (newly purchased from ATCC). Four rounds of phage selection were performed using cells of passage number 6 or less. The cells were then allowed to grow with media replacement every two to three days and passaging about once per week. Aliquots of the cells were collected and frozen at regular intervals (Passage numbers, 10, 20, 30, 40). A second parallel phage display selection was then performed at passage number 40.

Additionally, we utilized two different selection protocols. 1) An elution protocol, in which, phage library is incubated with cells, unbound phage washed away, and bound phage eluted. The eluted phage are then amplified and used in the next round of selection. Creates competition for binding sites-preferentially selects for high abundance biomarkers. 2) A depletion protocol, in which phage library is incubated with cells, unbound phage collected, and used in next round of selection. Should deplete the library of phage clones that bind to high abundance biomarkers. And finally, a negative selection was also performed. This selection identified phage that bind to wells with media, but no cells. Thus, we had a final tally of 8 separate parallel phage display selections/samples (see list of variables below). Each of these were then prepared for Next Generation Sequencing (NGS) and sequenced.

### List of Variables:

A. Cell Line
   a. LNCaP
   b. HEK 293
B. Phage Selection Method
   a. Elution (traditional)
   b. Depletion
C. Passage Number
   a. Low (passage 4 to 6)
   b. High (passage 40)

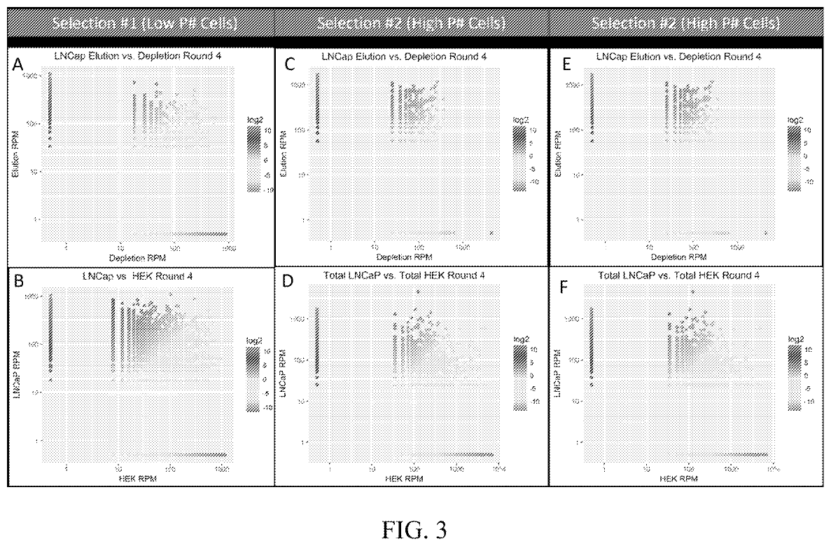

Resulting NGS data was then trimmed and translated into amino acid sequences (15 amino acid long). All peptide sequences identified within the negative selection (wells with media, but no cells) were deleted from all other sequences resulting from experimental selections. Below we provide an example of the phage display selection results. The particular example below is comparing the elution and depletion protocols on LNCaP cells-low passage number (FIG. 3A).
[symbol] 127,662 unique sequences (with RPM of 3 or more) (RPM=read per million)
[symbol] LNCaP-Elution Protocol
   A total of 32,106 sequences found binding to LNCaP using the Elution Protocol
   Of which 10,721 bound only to the LNCaP Cells in the Elution protocol (none found in HEK samples or depletion protocol)
   19,110 sequences with positive log 2 (+/−) ratio (More often found within Elution)
   12,996 sequences with neg log 2 (+/−) ratio (More often found within Depletion)
[symbol] LNCaP-Depletion Protocol
   For a total of 54,427 sequences found binding to LNCaP using the Elution Protocol
   Of which 13,273 bound only to the LNCaP Cells in the Elution protocol (none found in HEK samples or elution protocol)
   21,932 sequences with positive log 2 (+/−) ratio (More often found within Elution)
   32,495 sequences with neg log 2 (+/−) ratio (More often found within Depletion)
[symbol] 2,086 sequences were found within both the LNCaP selection protocols—but not the HEK293 cells.
[symbol] Of the 2,086 sequences-362 sequences had positive log 2 (+/−) ratios for both protocols

Other comparisons of parallel phage displays for various parameters are shown in FIGS. 3B-F.

Comparison of LNCaP to HEK-293 was utilized as an internal control to verify that the selection was not taken over by nonspecific target unrelated phages (TUPs) (1). And that to have a reference list of sequences that suggests the LNCaP specific peptide sequences identified may or may not be unique to LNCaP. While absence of an LNCaP peptide sequence from the HEK293 list of sequences is not a guarantee of cell line specificity: the presence of a peptide sequence within both lists is a guarantee of non-specificity. We then chose 40 unique phage clones from selections against both low and high passage numbers with which to carry forward.

Screening of selected phage clones. PHAGE clones were reconstructed and the clonal populations biotinylated. Biotinylated phage were used in a cell-based ELISA for 4 rounds of screening.

Results: 40 clones were screened using a qPCR-based method of quantifying cell bound phage. We did not find 24 phage clones of interest, instead we focused on two clones. Nonlimiting Example 3 describes the qPCR-based phage quantification of cell surface bound phage.

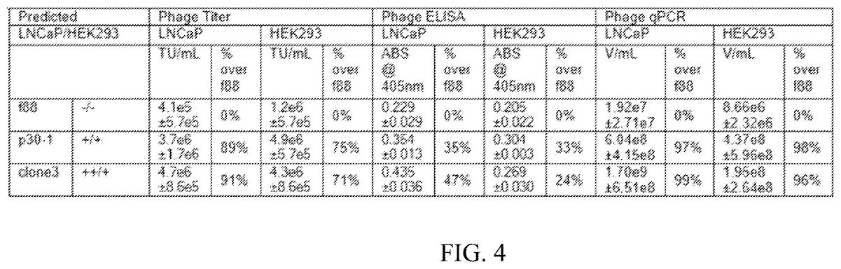

Details: The original proposal described the use of cell based ELISAs with biotinylated phage clones. However, this protocol was not sensitive enough and had high background (FIG. 4). The table in FIG. 4 shows that use of phage titer and qPCR protocols result in a much larger difference between f88 negative control and p30-1 (positive control) and clone3 (experimental) phage clones. The higher background in the cell-based phage ELISA is most likely due to the use of biotinylated phage. These biotinylated phages have multiple biotin molecules per phage virion. More specifically, the biotinylation of phage requires NHS ester-activated crosslinkers which react with primary amines: such as the terminal amine of coat protein VIII (˜3000 coat protein VIII per virion). This reaction results in multiple biotins per virion, which in turn leads to multiple streptavidin-HRP conjugate molecules per virion. Thus, it was not possible to truly quantify signal from the cell surface bound phage in the cell-based ELISA. Thus, we pivoted and began development of a qPCR-based phage quantification method. This method is described below and in nonlimiting Example 3.

Primers specific to phage vector/genome were generated and tested against LNCaP and HEK gDNA and total RNA for specificity. We then performed multiple experiments comparing the range of detection and repeatability. These primers were used to develop a qPCR protocol using whole phage in solution as the template. From this, a repeatable quantitative standard curve with a range of detection of 10³ to 10¹² v/mL was produced. This protocol has a limit of detection of 10³ v/mL and a limit of quantification of 10⁴ v/mL. In comparison, a cell-based ELISA protocol utilizing biotinylated phage has a lower limit of ˜105-10⁶ v/mL. And a cell binding assay using phage titer for quantification has a lower limit of ˜10⁴-10⁵ TU/mL. It is important to keep in mind that only about 5 to 10% of fUSE5 phage clones are infectious, thus this lower limit of ˜10⁴-10⁵ TU/mL is equivalent to ˜10⁶-10⁷ v/mL.

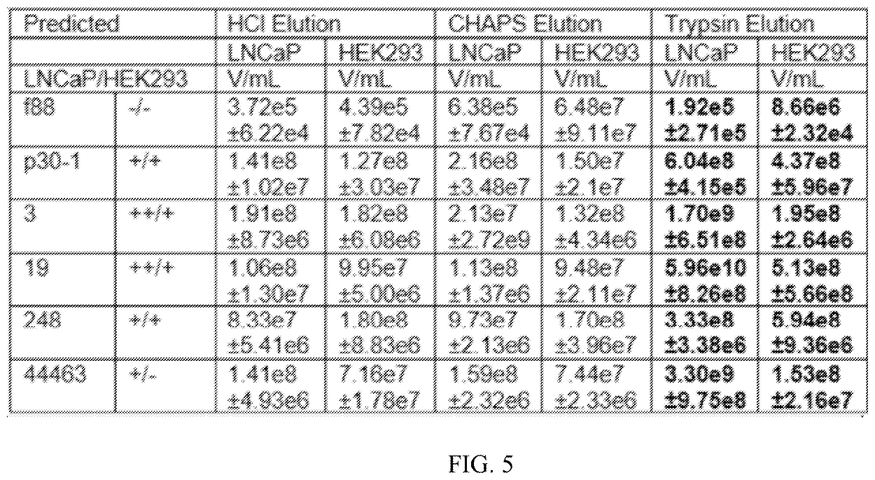

We next developed a protocol for the quantification of phage bound to the cell surface using qPCR (FIG. 5). To this end, we compared three different forms of elution for the removal of cell surface bound phage: acid elution (HCl), detergent elution (CHAPS), and trypsin elution. There are two different phage display vectors/phage genomes available for the display of 15 amino acid peptides on coat protein III of the fd type phage; fUSE5 and f3TR1. fUSE5 vector is the original display vector developed ˜1980 (2). While f3TR1 vector was modified by the addition of a trypsin cleavage site between the displayed peptide and the coat protein III (3). Use of trypsin release with the phage display vector, f3TR1, allows for a significant increase in sensitivity of detection of phage clones over previously utilized protocols (FIGS. 4 and 5). Additionally, the use of trypsin release phage display vector, f3TR1, helps to further reduce artifact from methodology. Acid and detergent elution protocols favor the elution of different types of peptides: i.e., Charged/hydrophilic vs nonpolar/hydrophobic. In summary, the qPCR and phage titer data are more quantitative than the cell-based ELISA. And the qPCR of cell surface bound phage is more sensitive and has a larger range of detection.

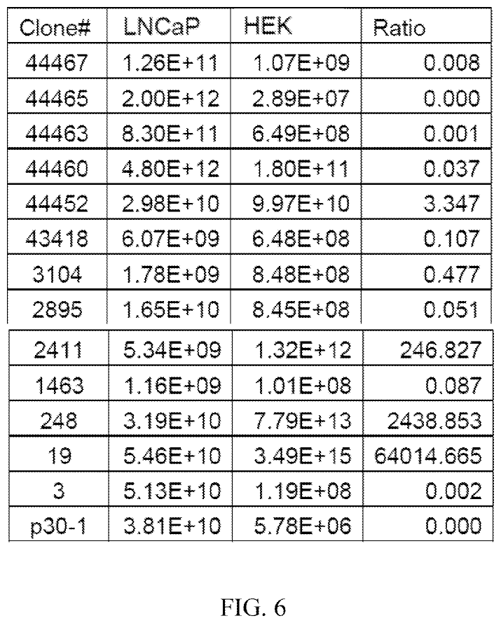

Thus, the 40 phage clones previously described were reconstructed within the trypsin cleavable phage display vector, f3TR1. Cell binding assays with the 40 clones revealed that 27 phage clones did not have saturable binding and thus did not result in Bmax or Kd. And 9 clones did not bind LNCaP cells with a high enough preferential binding. Cell line specificity is defined as the ratio of LNCaP:HEK293 EC50 values. In comparison, the data from cell binding assays for 4 clones (44467, 44465, 44463, and 3) did result in a ratio of LNCaP:HEK binding of 0.009 or less (FIG. 6).

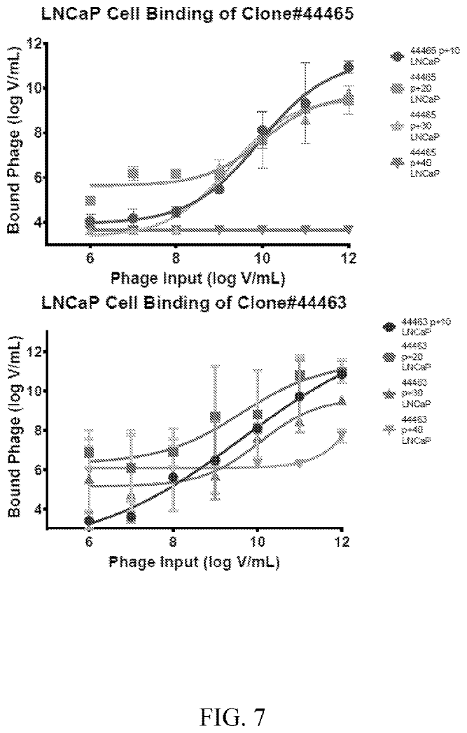

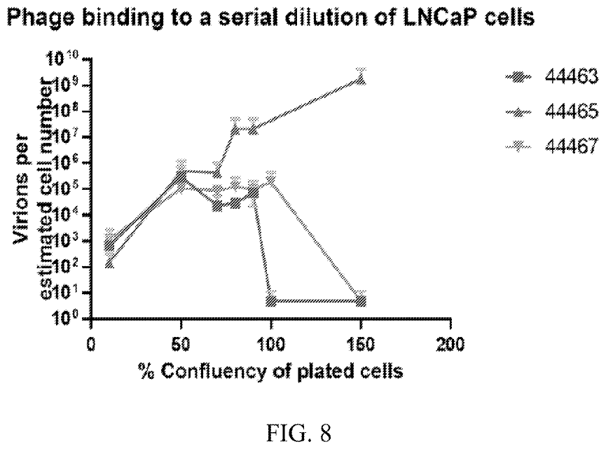

Of the 4 clones with saturable binding and preferential binding to LNCaP cells, the binding of 2 clones (44465 and 44463) were sensitive to the age of the cell lines (FIG. 7) and 3 clones (44467, 44465, and 44463) were sensitive to the confluency of cells at the time of cell binding (FIG. 8). LNCaP cells had previously been expanded and maintained for a set number of passages before being frozen (passage numbers 10, 20, 30, and 40). An aliquot of each were defrosted, expanded, and utilized within cell binding assays for the four phage clones of interest. Of these four clones, two revealed a reduction in cell binding in response to increasing passage number (FIG. 7).

FIG. 8 shows changes in cell surface binding of phage clones, 44463, 44465, and 44467, in response to cell confluency within the plate. The numbers of cells on the plate are estimated. There were significant differences in plating efficiency across the dilution series of cells, which in turn, prevented a manual cell count (unknown levels of lost cell numbers after plating). Thus, we tried using trypan blue to stain the fixed cells and use absorbance as a way to quantify cells. However, the presence of trypan blue at the end of the cell binding assay inhibited fluorescent detection necessary for qPCR of the phage clones in the solution. Next, we tried Cell Mask Deep Red with a fluorescent spectrum far removed from SYBR green. This cell stain works with fixed cells, survives the phage cell binding protocol, and gives repeatable RFU data within a certain range of cell numbers. However, the range linearity of the stain is not broad. Thus, the information presented here is an estimation.

Utilize CRISPR knockout library to identify gene(s) required for biomarker expression. The phage clones of interest described above were utilized in a loss of binding screen on LNCaP cells transfected using a genome wide CRISPR knockout library. Results: We successfully generated a library of LNCaP cells with a pooled, whole genome knockout CRISPR library from Cellecta (Mountain View, CA). Using the library, a loss of binding assay was performed, cells with reduced/loss of phage binding were sorted and collected. And finally, clusters of functionally related genes were identified. Nonlimiting Example 4 describes the phage display selection, screening, and CRISPR gene cluster identifications of selected phage clones.

### Details:

A loss of binding assay was performed on a mixed library of LNCaP cells; each transfected with a single CRISPR knockout cassette from the whole-genome knockout library. In short, phage clones positive (+) control (p30)-1), negative (−) control (f88), 44463, and 44465 phage clones were fluorescently labeled with AF488. Next, LNCaP cells at passage number 4 were first stably transfected with Cas9 with antibiotic selection. Then transfected with a whole genome knockout library of CRISPR/gRNA constructs, again with antibiotic selection and RFP internal positive control. Finally, successful transfection (RFP expression) was verified, and changes in phage cell binding quantified using cell flow cytometry. Next, LNCaP cells with no phage binding or reduced phage binding were sorted out of the LNCaP/CRISPR knockout library population (sorted out and collected RFP+/AF488-cell population). The gDNA of these individual cell populations were then isolated, purified, and used in nested PCR. The nested PCR first amplified gRNA identification sequences and then added Illuminia sequencing adaptors with index codes. The resulting NGS data was analyzed at the MU-IRCF where they organized the gRNA/gene ID by counts. Finally, the gene IDs were inputted into the STRING database version 11 (www.string-db.org) and groups of functionally related genes were identified and organized.

The positive control, p30-1, was selected and affinity maturated against purified Thomsen-Friendreich (TF) Antigen, a disaccharide carbohydrate tumor antigen (4-7). It is a Core-1 (Galβ1-3GalNAcα-1Thr/Ser) structure of O-glycans (7). The TF antigen is a confirmed tumor antigen, though the exact pathway of synthesis of it is unknown, it is generally thought that TF antigen is usually hidden within longer carbohydrate chains on normal tissues (8). The cell population analyzed for the loss of binding to the p30-1 clone revealed 2 of the 6 known mucin proteins within prostate, MUC3A and MUC4 (FIG. 9) (9). Both are membrane bound and MUC4 is known to display TF (10). Interestingly, there were 4 different β-1,3-galactosyltransferases (B3GALT1, -2, -4, and -5). These enzymes transfer galactose from UDP-galactose to substrates with a terminal β-GlcNAc residue; required for the first step after the Core-1 formation (4. 6). From there the Core-1 O-glycosylation biochemical pathways exist in equilibrium between sulfotransferases, sialyltransferases, and Fuc-transferases. While not wishing to be bound by theory, we hypothesize that the decrease in p30-1 binding on cells with ST3GAL4, ST6GAL2, FUT2, and FUT8, knockouts would be due to further perturbations within the O-glycosylation equilibrium potentially pushing glycosylation towards Core-2 (Core-1 with branched (1-6GlcNAc additions) (4). Importantly, the C1GALT1C1 is a molecular chaperone with known influence on the synthesis of TF antigen (11).

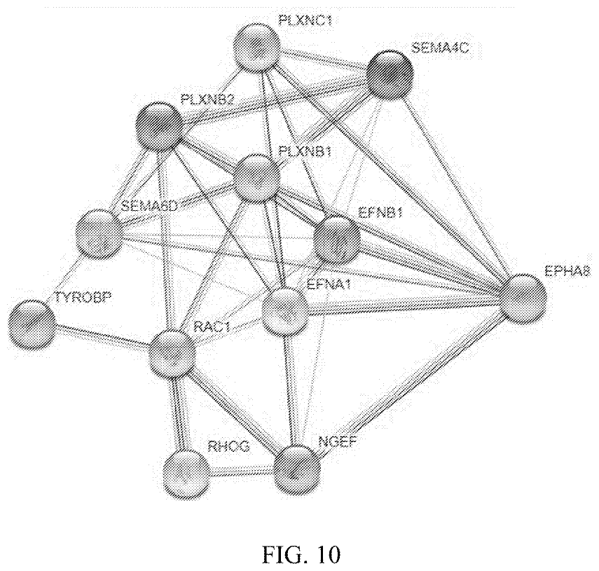

Analysis of clone 44463 loss of binding data revealed a cluster of functionally related genes in the semaphorin, plexin, ephrin/eph receptor neuronal guidance system/axon outgrowth pathways (FIG. 10). However, this system has recently been linked to cancer progression, angiogenesis, and metastasis (12, 13). The string database reports 7 experimentally determined interactions (out of the 12 total genes) between EPHA8 (Ephrin type-A receptor 8) and various ephrins, semaphorins, and plexins. Suggesting that EPHA8 may be the center of this cluster. These pathways are known to influence/control cytoskeleton remodeling through the GTPases Rho and Rac (14-17).

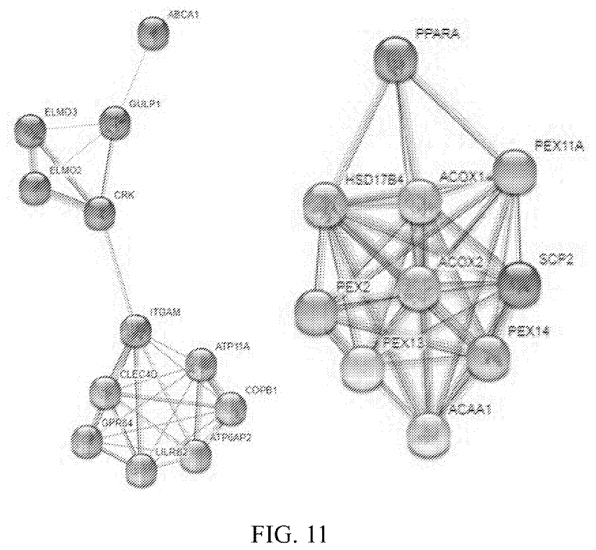

Analysis of clone 44465 data revealed 2 related clusters (FIG. 11). The larger cluster contains 10 genes (FIG. 11). Nine were confirmed for expression within peroxisomes and the last (PPARA=peroxisome proliferator-activated receptor alpha) is a ligand-activated transcription factor and key regulator of lipid metabolism (18, 19). Four genes are members of the peroxisomal biogenesis factor (PEX) family: specific for peroxisome proliferation and movement (20). While the ACOX1/2, SCP2, ACAA1, and the HSD17B4 genes are all involved in metabolism of fatty acids/lipids (21-23). The smaller cluster contains genes required for phagocytosis (green circles), genes associated with membrane rafts (purple circles), and genes associated with vesicle membranes (red circles) (FIG. 11) (24-30). We, thus, hypothesize that 44465 binds to a type of lipid.

Results Summary: We have generated enough data for proof of concept for two phage clones, 44463 and 44465. These two clones were selected, identified, and characterized as proposed.

### BIBLIOGRAPHY

1. Newton-Northup J, Duetscher S L. Contending with target unrelated peptides from phage display. J Mol Imaging Dynam. 2012; 2 (2). doi: 10.4172/2155-9937.1000e101.
2. Smith G P. Filamentous fusion phage: novel expression vectors that display cloned antigens on the virion surface. Science. 1985; 228 (4705): 1315-7. Epub 1985 Jun. 14. doi: 10.1126/science.4001944. PubMed PMID: 4001944
3. Thomas W D, Smith G P. The case for trypsin release of affinity-selected phages. Biotechniques. 2010; 49 (3): 651-4. Epub 2010 Sep. 22. doi: 10.2144/000113489. PubMed PMID: 20854266.
4. Brockhausen I. Pathways of O-glycan biosynthesis in cancer cells. Biochim Biophys Acta. 1999; 1473 (1): 67-95. Epub 1999 Dec. 2. doi: 10.1016/s0304-4165 (99) 00170-1. PubMed PMID: 10580130.
5. Brockhausen I. Mucin-type O-glycans in human colon and breast cancer: glycodynamics and functions. EMBO Rep. 2006; 7 (6): 599-604. Epub 2006 Jun. 3. doi: 10.1038/sj.embor.7400705. PubMed PMID: 16741504: PMCID: PMC1479595.
6. Brockhausen I, Yang J M, Burchell J, Whitehouse C, Taylor-Papadimitriou J. Mechanisms underlying aberrant glycosylation of MUC1 mucin in breast cancer cells. Eur J Biochem. 1995; 233 (2): 607-17. Epub 1995 Oct. 15. doi: 10.1111/j.1432-1033.1995.607_2.x. PubMed PMID: 7588808.
7. Hanisch F G, Baldus S E. The Thomsen-Friedenreich (TF) antigen: a critical review on the structural, biosynthetic and histochemical aspects of a pancarcinoma-associated antigen. Histol Histopathol. 1997; 12 (1): 263-81. Epub 1997 Jan. 1. PubMed PMID: 9046061.
8. Goletz S, Cao Y, Danielczyk A, Ravn P, Schoeber U, Karsten U. Thomsen-Friedenreich antigen: the “hidden” tumor antigen. Adv Exp Med Biol. 2003; 535:147-62. Epub 2004 Jan. 13. doi: 10.1007/978-1-4615-0065-0_10. PubMed PMID: 14714894.
9. Russo C L, Spurr-Michaud S, Tisdale A, Pudney J, Anderson D, Gipson I K. Mucin gene expression in human male urogenital tract epithelia. Hum Reprod. 2006; 21 (11): 2783-93. Epub 2006 Sep. 26. doi: 10.1093/humrep/del164. PubMed PMID: 16997931: PMCID: PMC2893033.
10. Brocke C, Kunz H. Synthetic Tumor-Associated Glycopeptide Antigens from the Tandem Repeat Sequence of the Epithelial Mucin MUC4. Synthesis. 2004; 4:525-42. doi: 10.1055/s-2004-815962.
11. Liu J, Xu F, Li J, Jiang H. Overexpression of Cosmc suppresses cell migration and invasion in different subtypes of breast cancer cells via Tn and T glycans. Biosci Rep. 2020; 40 (6). Epub 2020 May 27. doi: 10.1042/BSR20191062. PubMed PMID: 32452513: PMCID: PMC7300286.
12. Nasarre P, Potiron V, Drabkin H, Roche J. Guidance molecules in lung cancer. Cell Adh Migr. 2010; 4 (1): 130-45. Epub 2010 Feb. 9. doi: 10.4161/cam.4.1.10882. PubMed PMID: 20139699; PMCID: PMC2852570.
13. Neufeld G, Sabag A D. Rabinovicz N, Kessler O. Semaphorins in angiogenesis and tumor progression. Cold Spring Harb Perspect Med. 2012; 2 (1): a006718. Epub 2012 Feb. 9. doi: 10.1101/cshperspect.a006718. PubMed PMID: 22315716; PMCID: PMC3253028.
14. Hota P K, Buck M. Plexin structures are coming: opportunities for multilevel investigations of semaphorin guidance receptors, their cell signaling mechanisms, and functions. Cell Mol Life Sci. 2012; 69 (22): 3765-805. Epub 2012 Jun. 30. doi: 10.1007/s00018-012-1019-0. PubMed PMID: 22744749.
15. Le A P, Huang Y, Pingle S C, Kesari S, Wang H, Yong R L, Zou H, Friedel R H. Plexin-B2 promotes invasive growth of malignant glioma. Oncotarget. 2015; 6 (9): 7293-304. Epub 2015 Mar. 13. doi: 10.18632/oncotarget.3421. PubMed PMID: 25762646; PMCID: PMC4466685.
16. Leung C, Liu Y, Lu X, Kim M, Drysdale T A, Feng Q. Rac1 Signaling Is Required for Anterior Second Heart Field Cellular Organization and Cardiac Outflow Tract Development. J Am Heart Assoc. 2015; 5 (1). Epub 2016 Jan. 2. doi: 10.1161/JAHA.115.002508. PubMed PMID: 26722124: PMCID: PMC4859369.
17. Taddei M L, Parri M, Angelucci A, Onnis B, Bianchini F, Giannoni E, Raugei G, Calorini L, Rucci N, Teti A, Bologna M, Chiarugi P. Kinase-dependent and -independent roles of EphA2 in the regulation of prostate cancer invasion and metastasis. Am J Pathol. 2009; 174 (4): 1492-503. Epub 2009 Mar. 7. doi: 10.2353/ajpath.2009.080473. PubMed PMID: 19264906; PMCID: PMC2671379.
18. Segawa Y, Yoshimura R, Hase T, Nakatani T, Wada S, Kawahito Y, Kishimoto T. Sano H. Expression of peroxisome proliferator-activated receptor (PPAR) in human prostate cancer. Prostate. 2002; 51 (2): 108-16. Epub 2002 Apr. 12. doi: 10.1002/pros. 10058. PubMed PMID: 11948965.
19. Collett G P, Betts A M, Johnson M I, Pulimood A B, Cook S, Neal D E, Robson C N. Peroxisome proliferator-activated receptor alpha is an androgen-responsive gene in human prostate and is highly expressed in prostatic adenocarcinoma. Clin Cancer Res. 2000; 6 (8): 3241-8. Epub 2000 Aug. 24. PubMed PMID: 10955810.
20. Okumoto K, Tamura S, Honsho M, Fujiki Y. Peroxisome: Metabolic Functions and Biogenesis. Adv Exp Med Biol. 2020; 1299:3-17. Epub 2021 Jan. 9. doi: 10.1007/978-3-030-60204-8_1. PubMed PMID: 33417203.
21. Chen J, Chen J, Fu H, Li Y, Wang L, Luo S, Lu H. Hypoxia exacerbates nonalcoholic fatty liver disease via the HIF-2alpha/PPARalpha pathway. Am J Physiol Endocrinol Metab. 2019; 317 (4): E710-E22. Epub 2019 Aug. 21. doi: 10.1152/ajpendo.00052.2019. PubMed PMID: 31430204.
22. Thompson J, Lepikhova T, Teixido-Travesa N, Whitehead M A, Palvimo J J, Janne O A. Small carboxyl-terminal domain phosphatase 2 attenuates androgen-dependent transcription. EMBO J. 2006; 25 (12): 2757-67. Epub 2006 May 26. doi: 10.1038/sj.emboj. 7601161. PubMed PMID: 16724108: PMCID: PMC1500849.
23. Zha S, Ferdinandusse S, Hicks J L, Denis S, Dunn T A, Wanders R J, Luo J, De Marzo A M, Isaacs W B. Peroxisomal branched chain fatty acid beta-oxidation pathway is upregulated in prostate cancer. Prostate. 2005; 63 (4): 316-23. Epub 2004 Dec. 16. doi: 10.1002/pros.20177. PubMed PMID: 15599942.
24. Beron W. Mayorga L S. Colombo M I. Stahl P D. Recruitment of coat-protein-complex proteins on to phagosomal membranes is regulated by a brefeldin A-sensitive ADP-ribosylation factor. Biochem J. 2001; 355 (Pt 2): 409-15. Epub 2001 Apr. 4. doi: 10.1042/0264-6021:3550409. PubMed PMID: 11284728: PMCID: PMC1221752.
25. Feller S M. Crk family adaptors-signalling complex formation and biological roles. Oncogene. 2001; 20 (44): 6348-71. Epub 2001 Oct. 19. doi: 10.1038/sj.one.1204779. PubMed PMID: 11607838.
26. Gumienny T L, Brugnera E, Tosello-Trampont A C, Kinchen J M, Haney L B, Nishiwaki K, Walk S F, Nemergut M E, Macara I G, Francis R, Schedl T, Qin Y, Van Aelst L, Hengartner M O, Ravichandran K S. CED-12/ELMO, a novel member of the CrkII/Dock180/Rac pathway, is required for phagocytosis and cell migration. Cell. 2001; 107 (1): 27-41. Epub 2001 Oct. 12. doi: 10.1016/s0092-8674 (01) 00520-7. PubMed PMID: 11595183.
27. Hamon Y, Trompier D, Ma Z, Venegas V, Pophillat M, Mignotte V, Zhou Z, Chimini G. Cooperation between engulfment receptors: the case of ABCA1 and MEGF10. PLOS One. 2006; 1:e120. Epub 2007 Jan. 6. doi: 10.1371/journal.pone.0000120. PubMed PMID: 17205124; PMCID: PMC1762421.
28. Jaumouille V, Waterman C M. Physical Constraints and Forces Involved in Phagocytosis. Front Immunol. 2020; 11:1097. Epub 2020 Jul. 1. doi: 10.3389/fimmu.2020.01097. PubMed PMID: 32595635: PMCID: PMC7304309.
29. Lishko V K, Moreno B, Podolnikova N P, Ugarova T P. Identification of Human Cathelicidin Peptide LL-37 as a Ligand for Macrophage Integrin alphaMbeta2 (Mac-1, CD11b/CD18) that Promotes Phagocytosis by Opsonizing Bacteria. Res Rep Biochem. 2016; 2016 (6): 39-55. Epub 2016 Dec. 19. doi: 10.2147/rrbc.s107070. PubMed PMID: 27990411: PMCID: PMC5157691.
30. Park M, Raftery M J, Thomas P S, Geczy C L, Bryant K, Tedla N. Leukocyte immunoglobulin-like receptor B4 regulates key signalling molecules involved in FcgammaRImediated clathrin-dependent endocytosis and phagocytosis. Sci Rep. 2016; 6:35085. Epub 2016 Oct. 12. doi: 10.1038/srep35085. PubMed PMID: 27725776; PMCID: PMC5057125.

### Example 3

Introduction: In 1985. Dr. George Smith pioneered phage display technology, which is a powerful combinatorial technique used to select and identify peptide- and antibody-based ligands. Phage display libraries of up to 10⁹ unique clones are assembled from genetically modified virions that display a foreign polypeptide on the N-terminus of coat proteins. Polypeptide-based ligands with high specificity and affinity for a molecular target may then be selected from the phage library through multiple rounds of competitive affinity selections. In this manner, phage display technology has been used to develop peptides, antibodies, and antibody fragments with excellent binding properties. These ligands have been used to target biomolecules both in vitro and in vivo for drug delivery, imaging, and therapeutic purposes. Here, we focus on phage libraries developed by George Smith that display 6 or 15 amino acid random peptide on the surface of coat protein III. Specifically, the first generation fUSE5 peptide libraries and the second generation f3TR1 library.

In traditional random peptide phage display selection protocols, multiple rounds of affinity selections are utilized, in which the library is incubated with the molecular target and bound phage are collected by elution. After each round, the phage clones are amplified and then compete for binding within subsequent rounds of selection. After each round, DNA amplicons corresponding to the foreign polypeptide insert are typically sequenced to evaluate if the phage population is undergoing sufficient selection and to detect overgrowth of known target unrelated phages (TUPs). After the last selection round, deep next generation sequencing (NGS) of DNA amplicons and bioinformatic analysis are carried out to identify, translate and sort selected peptide ligands.

While the phage display selection rounds are typically completed in a time-efficient manner, the subsequent screening of selected peptide ligands is often the rate-limiting step in an otherwise high-throughput technology. Screening of soluble peptide ligands require extensive and expensive synthesis, purification, and validation steps using solid-phase synthesis, high-pressure liquid chromatography (HPLC), and mass spectrometry. To the contrary, screening of the foreign peptide-displaying phage is more cost-effective as these can be assembled and propagated using straightforward molecular cloning procedures and isolation by precipitation and centrifugation. However, the screening process of both soluble ligands and phage particles has typically taken months to years to perform. Analysis of binding interactions is often carried out by enzyme-linked immunosorbent assay (ELISA)-based methods, surface plasmon resonance (SPR), and dynamic light scattering (DLS). Methods such as ELISA that use fluorescent or colorimetric measurements suffer from low sensitivity, while SPR and DLS often have low throughput and/or expensive to use.

Researchers are thereby often limited to selecting just a few ligands to screen and analyze, which may prevent discovery of important polypeptides. To address this challenge, we have developed a method of high-throughput cloning, propagation and screening of phage that is both cost- and time-efficient.

The methods described here are post-phage display selection and sequence analysis. We have streamlined the phage reconstruction and screening process needed after NGS sequencing of phage display selections. Here a seven-day work flow is illustrated with end products of clonal phage populations and known Kd values for immobilized target.

Experimental Design: The phage clones used in this protocol are derived from the f3TR1 peptide library that is based on genetically modified fd phage that display a foreign 15-mer peptide on coat protein III (cpIII) with a trypsin cleavage site between the peptide and the N-terminus of cpIII. The phage genome includes a tetracycline resistance gene (tetA) and the entire wild type (WT) fd phage genome (GenBank: HM355479. 1). Thus, these phages carry the genetic information necessary for infection of and propagation in E. coli. Although our protocol utilizes phage that are fully capable of infection and propagation, phagemid particles derived from other phage display libraries may also be used if an appropriate helper phage is used to ensure proper propagation.

This protocol describes the reconstruction (cloning) of individual phage clones that were identified via phage display technology, followed by the propagation, isolation, and screening of ligand binding interactions. Traditionally, phage eluted/collected within the final round of selection were plated and individual colonies picked, propagated, and the DNA sequenced. This allowed researchers to save aliquots of phage clones for future screening experiments. However, with the development of NGS the eluted phage sample is identified via bioinformatic analysis only. Consequently, each phage clone of interest must be reconstructed to obtain a physical clone. Here we describe a protocol for clone reconstruction and screening amenable to a high throughput workflow using a 96-sample format (FIG. 12).

For reconstruction of phage clones, the individual DNA sequences corresponding to the foreign peptide inserts are synthesized, hybridized, and ligated into the coat protein III gene for display on the N terminus. Use of fd-tet phage vector digested with SfiI allowed for directional cloning, thus the hybridized oligoes were designed with single stranded overhangs matching those of the digested vector. The restriction enzymes used to digest the phage genome must be determined for each experiment if using a different vector. The ligated vectors/clones are then transformed into electrocompetent MC1061 (Lucigen Corp, Middleton, WI. USA) E. coli cells by electroporation. Alternatively, transformation using the heat-shock method may be used if the vector and E. coli strain are compatible.

The fUSE5 and f3TR1 phage vectors are low copy number vectors. Specifically, the minus-strand synthesis of the genome is disrupted (Smith 1988). This in turn reduces copy number of the viral DNA and significantly reduces cell killing of infected E. coli cells. Thus, infected E. coli cultures can be grown for longer periods of time to produce higher numbers of fd-tet phage, in comparison to other phage vectors. Propagation of phage clones often involves growing liter volumes of E. coli followed by several steps of isolation and purification, which leads to phage quantities in the 1013-1014 virion range. The typical large volume of E. coli cultures complicates the isolation of more than a few clones per week. Instead, phage may be propagated in 1 mL cultures on deep 96-well plates, from which the clones can be directly isolated. This provides sufficient virions for subsequent analysis of binding properties. A centrifuge that can accommodate a 96 well plate is required. Alternatively, the phage cultures may be transferred to microcentrifuge tubes for centrifugation. First, the E. coli cells are cleared from the growth media via centrifugation. Next, the phage are precipitated from the growth media using polyethylene glycol (PEG)/NaCl followed by centrifugation. The isolation protocol described here was modified from the method by Dr. George Smith [1]. Following isolation, the concentration (virions per mL; V/mL) of individual phage clones may be determined spectrophotometrically (A269 and A320).

Screening of phage binding interactions using quantitative polymerase chain reaction (qPCR) is sensitive, relatively cost effective, and can be carried out in a high-throughput manner. Here, we used a qPCR assay to evaluate the binding of selected phage clones to various human cell lines (FIG. 12). Primers against cpIII of the fd phage were used to ensure consistent hybridization kinetics between different phage clones. A standard curve of phage was generated to relate the cycle threshold (Ct) to V/mL. Additionally, it allowed for the probing of the range of detection, linearity, and repeatability of the new protocol. Next, different human cell lines were plated on 96-well plates and grown overnight in appropriate media. For protein and other molecular antigens, these may instead be immobilized on suitable plates according to the manufacturer's instructions. The individual phage clones were then incubated with the cells to allow binding, which was followed by washing, and elution. For phage clones constructed within the f3TR1 vector, trypsin elution is most efficient. Other phage vectors including fUSE5, the elution of bound phage may be accomplished by interrupting the non-covalent binding interactions via changes in the pH or use of detergent (ref). However, the pH must be adjusted prior to qPCR to accommodate the polymerase (the optimal pH of the polymerase should be determined from the manufacturer's instructions). The pH may be adjusted using 2-amino-2-(hydroxymethyl)-1,3-propanediol. While phage are often eluted using detergents in various assays, caution should be taken using detergents when conducting qPCR. Ionic detergents are highly inhibitory for the PCR reaction due to denaturation of the polymerase. Non-ionic detergents may be used at lower concentrations without inhibiting the polymerase. Nevertheless, low detergent concentrations may not be sufficient to elute bound phage [2]. Following elution, the collected phage may be used directly in qPCR reactions to determine the Ct-value, which subsequently is used to calculate the V/mL using the standard curve. In this experiment, the calculated V/mL was directly related to the binding affinity of the polypeptide-displaying phage for the human cell lines.

### Day 1: Preparation of Phagemid Phage Genome

Vector digest

Digest f3TR1 vector with SfiI. Follow manufacturer's directions.

In short, incubate 1 μg phage DNA with 20 units SfiI enzyme in provided buffer system at 50° C. for 15 minutes.

CIP treatment to dephosphorylate the 5′ and 3′ DNA ends. This is done to prevent re-cyclization and increase shelf-life of digested phage genome.

In short, add 10 units of CIP to digestion reaction tube and incubate at 37° C. for 10 minutes. Followed by heat-inactivation of enzymes at 80° C. for 2 minutes.

Remove stuffer region oligo and enzymes via PCR/DNA cleanup kit.

Note: alternative methods to remove the stuffer region are dialysis or gel purification of the vector.

Hybridize/anneal DNA oligos

Prior to Day 2: Design DNA oligos

SfiI enzyme leaves sticky ends

Prior to Day 2: Order DNA oligos

Option 1: order prehydbridized oligos

Mix purchased oligoes in a 1:1 molar ratio in room temperature STE buffer.

Heat samples in water bath or in heat block to 95° C. for 10 minutes.

Leave samples in water bath/on heat block but turn off heat.

Allow the samples to slowly cool back to room temperature.

Stable in storage for short term (about 2 days) at 4° C.

Note: if necessary, oligoes can be reheated and reannealed.

Annealed Oligo Cloning

Perform the ligation reaction as required by the manufacturer.

Note: It is important to ensure that the provided ligation reaction buffer contains 1 mM ATP. If not, the buffer should be supplemented. ATP supplementation is necessary due to the previous step of CIP dephosphorylation.

In short, prepare reaction in provided buffer with 400 units of T4 DNA Ligase, digested phage genome, and hybridized oligo insert.

Note: Depending upon the size of the oligo insert, mix the annealed oligo and digested vector in a molar ratio of 1:10 up to 1:20 (vector:insert).

Incubate at room temperature for 20 minutes. Followed by heat inactivation at 65° C. for 10 minutes.

### Day 2: Electroporation

Set up electroporator for 1250V, with 400Ω×25 μF (which usually results in a time constant of about 10 msec-depending upon the system).

Prepare SOC with 0.2 μg/mL tetracycline.

Note: This low concentration of tetracycline is not enough to kill the E. coli, it's presence is intended to induce expression of the phage genome's tetracycline resistance gene within the 45 minute outgrowth (see below).

Ensure NZY/Tet plates are ready and clean with no contaminating growth.

Bucket of ice with prechilled DNA samples and empty cuvettes.

Layer 25 μL of 25% glycerol on the bottom of the chilled cuvette ensuring that there are no bubbles.

Defrost MC1061 E. coli cells and transfer 25 μL to fresh tube with 1 to 5 μL ligated DNA and gently mix by pipetting up and down. Careful not to introduce bubbles.

Carefully layer the E. coli/DNA mix on top of the glycerol inside of the cuvette.

Quickly place the cuvette in the electroporator and “shock” the sample only once (only one pulse).

Quickly pipette 0.5 mL SOC/Tet into cuvette and transfer entire volume to clean 15 mL culture tube. Followed immediately by rinsing the cuvette with a further 0.5 mL SOC/Tet which is also added to the same 15 mL culture tube.

Place the 15 mL culture tube on a shaker incubator at 37° C. and 250 rpm for 45 minutes.

Working quickly with small batches of about 4 to 6 samples helps to ensure the maintenance of low temperature within the first couple of steps. Speed also helps ensure the recovery of the E. coli by placing them within the shaker incubator as soon as possible after the electroporation.

At the end of the 45 minute out growth/gene expression period the cultures should be plated on NZY/Tet plates. It is optimal to plate various dilutions of the liquid culture (1×, 0.5×, 0.1×). and incubate at 37° C. overnight.

### Day 3: Phage Propagation

Inspect the NZY/Tet plates with transfected E. coli.

You will need clearly isolated colonies with no other colonies touching. If the colonies are too dense or have grown into a lawn then you must pause this work flow and streak another plate. In short, using an autoclaved toothpick or sterile inoculating loop lightly touch the overgrown E. coli. Transfer the E. coli to a new clean plate by lightly streaking multiple lines across the plate.

If there are no colonies or very few colonies (with no insert) then (1) check the buffer of ligation reaction. (2) use a PCR/DNA cleanup kit to remove ligase enzyme and/or inappropriate buffer. (3) increase volume of ligated DNA used in electroporation (up to 5 μL). Or (4) repeat process verifying appropriate conditions for enzyme digest, CIP treatment, and/or oligo annealing.

Add 1 mL NZY/kanamycin/tetracycline to the wells of a deep (2 mL capacity) 96-well plate.

Using an inoculation loop or autoclaved toothpick, pick a single isolated E. coli colony from NZY/Tet plate and swirl in the 1 mL NZY media.

Cover the plate using breathable sealing tape making sure that each well is properly sealed to avoid cross contamination.

Incubate the plate on a shaker at 250 rpm overnight at 37° C.

Day 4: Separation of Phage from E. coli and Sequence Verification
Separation of E. coli and Phage Virions

Centrifuge the deep 96 well plate at 3000×g for 5 min (xxx). Alternatively, the overnight cultures may be transferred to microcentrifuge tubes. The centrifugation speeds and incubation times will remain the same.

Carefully, transfer the supernatant which contains the phage virions onto a fresh deep 96 well plate being cautious to not disturb the pellet of E. coli cells.

Cover the new 96-well plate of supernatant/phage virions with a non-breathable sealing tape and store at 4° C.

Note: The collected supernatants contain phage of unverified sequence. Once each colony/clone is sequence verified these supernatants will be utilized to infect a different strain of E. coli for the further propagation of phage.

### Plasmid DNA Preparation and Sequence Verification

The phagemid/phage genome can be treated as a large bacterial plasmid. Thus, using plasmid DNA purification kit isolate and purify the DNA of each bacterial pellet.

Use a spectrophotometer to quantify DNA of each sample.

Submit DNA samples to a DNA sequencing service (traditional Sanger sequencing is sufficient) with an appropriate primer. We have utilized multiple primers successfully. Refer to service provider for appropriate primer concentration, length, Tm, etc.

TABLE 1
Primers

| Primer | | | | |
|---|---|---|---|---|
| Name | Sequence | Length | Tm | Sequencing direction |
| fUSE5 | 5′-TGAATTTTCTGTATGAGG-3′ | 18 bp | 43.6° C. | Antisense or Reverse |
| primer | (SEQ IN NO: 6) | | | Complement |
| Long fUSE | 5′- | 20 bp | 52.5° C. | Antisense or Reverse |
| | CGTTACGCTAACTATGAGGG-3′ | | | Complement |
| | (SEQ ID NO: 7) | | | |
| LeftfUSE | 5′-TTTTGGAGATTTTCAACGTG- | 20 bp | 49.1° C. | Sense or In-line |
| | 3′ (SEQ ID NO: 8) | | | |

Add 150 μL PEG/NaCl and shake the plate gently (50 rpm) for 2 h at 4° C. to precipitate the phage particles.

PAUSE STEP The phage may be precipitated overnight at 4° C.

Centrifuge at 5000×g for 15 min at 4° C. Carefully discard the supernatant.

Add 1 mL TBS and gently resuspend the phage pellet.

CRITICAL STEP The phage pellet should be resuspended gently by pipetting slowly to avoid cross contamination of phage between the wells. Ideally cover the other wells when pipetting.

Centrifuge at 5000×g for 10 min. Carefully collect the supernatant and transfer to a fresh deep 96 well plate.

Add 150 μL PEG/NaCl and shake the plate gently (50 rpm) for 20 min at 4° C. to precipitate the phage particles.

PAUSE STEP The phage may be precipitated overnight at 4° C.

Centrifuge at 5000×g for 15 min at 4° C. Carefully discard the supernatant.

Add 100 μL TBS and gently resuspend the phage pellet making sure to avoid cross contamination.

Store the resuspended phage at 4° C.

PAUSE STEP The phage may be stored at 4° C. for up to a month and still be used in binding interaction studies. Phage can be stored long term (years) at 4° C. and maintain their infectivity of E. coli (ref). Phage that are stored long term should be re-propagated in E. coli to ensure that the displayed polypeptide ligand is intact.

### Measurement of Phage Concentration (V/mL)

Dilute the isolated phage 10-100 times in TBS. For example, for a 100-time dilution add 1 μL phage to 99 μL TBS in a well on a 96 well plate suitable for UV/Vis spectrophotometry.

Add 100 μL TBS to a separate well as a blank measurement.

Measure the absorbance at 269 nm and 320 nm.

Calculate the V/mL using the following equation:

$$V / mL = ( {A}_{269} - {A}_{320} ) \cdot 6 \times {10}^{16} ) / ( number of bases )$$

The number of bases is different for each type of phage used. For the fd phage derived from the f3TR1 library the number of bases is 8958.

### Cell Culture

Propagate the human cell lines in complete medium at 37° C. and 5% CO2 until 80-90% confluency. Here, hTERT-HPNE, Mia Paca-2, SKOV-3, and LNCaP cells were used. Other cell lines may be used instead.

Plate 10⁵ cells per well on a 96 well plate in complete medium and incubate overnight at 37° C. and 5% CO2. The number of cells per well may be different for other cell lines. Ideally, the number of cells to be used should lead to 80-90% confluence overnight.

Fixed cells.

### RT-qPCR

Dilute the phage to 1010 V/mL in TBS.

Add 100 μL of the 1010 V/mL phage into each well of plated cells and incubate for 1 hr at room temperature.

Gently aspirate the phage solution and wash the cells three times with TBS. Note: Depending on the binding affinity of the polypeptide ligand and the nature of the antigen, washing may be done using 0.05% Tween-20 in TBS. This step may need to be optimized for each antigen studied.

Elute the bound phage by addition of trypsin.

In at least triplicates, use 5 μL eluted phage as input and use qPCR to quantify the relative binding of each phage to each cell line.

### BIBLIOGRAPHY

1. Smith, G. P. G. P. Smith Lab Homepage. 2018 [cited 2018 06012019]: Available from: www.biosci.missouri.edu/smithGP/.
2. Weyant. R. S., P. Edmonds, and B. Swaminathan, Effect of ionic and nonionic detergents on the Taq polymerase. Biotechniques, 1990. 9 (3): p. 308-9.

### Example 4

Specific Aims: Development of a methodology able to characterize aspects of cellular phenotype within cells by dually targeting and assessing both mRNA transcripts (i.e., RNA transcripts) and cell surface biomarkers (i.e., biomarker expression). This methodology can be developed into a cheap and easily accessible kit. In short, in concert with Short Tandem Repeat analysis, this methodology can enable probing three tiers of cell biology; DNA sequence, RNA transcripts, and biomarker expression. This methodology can quantify non-traditional phenotypic markers in a sensitive, quantitative, easy-to-use, and cost-effective manner. Bacteriophage (ø) expressing multiple copies of a targeting peptide are used to bind cell surface biomarker. The ssDNA genome of the o, with the unique foreign peptide genetic sequence, can then be used to translate protein/carbohydrate/lipid biomarkers into a PCR based quantifiable signal. Quantification of bound ø via qPCR is paired to qRT-PCR analysis of correlating mRNA transcripts. An outcome of this project can include a panel of unique ø clones for incubation with any cells. After free ø are washed away and total nucleic acid isolated, mRNA transcripts and ø clones are quantified via qRT-PCR/qPCR. The resulting Ct values can then be normalized and compared to a database containing validated levels of both mRNA and biomarker expression. Analysis of a matrix of sentinel biomarkers/o clones/mRNA transcripts can give researchers cost-effective, easy access to information about a cell line's phenotypic profile. The ability to directly compare changing mRNA and cell surface biomarker levels over the course of time can provide useful information about altered biochemical pathways. In this way, we can use a single common protocol to probe an array of different cell types/lines. It is envisioned that experiments will be reported along with the expression levels of the sentinel mRNA/biomarkers within that cell line's matrix. This information can identify aged or inappropriately cultured cells, evaluate published data, and aid in production of reproducible results. In some embodiments, a panel of ø clones and corresponding mRNA transcripts able to characterize specific aspects of cellular phenotype of all ATCC prostate cell lines are developed. The method can 1) verify phenotypic profile of the cells, 2) verify correct culturing conditions and/or age of cells, and 3) be able to discern “drifting” within the cells. This panel of ø clones/mRNA transcripts can show feasibility, and the developed method will then be utilized in the future for many different cell types.

In these studies, screening for saturable binding of ø clones to 2D cultured LNCaP cells was performed. Next, the cell surface binding of these ø clones to cells that were grown in inappropriate culture conditions was compared to the results of 8 measures of cell health. In these studies, ø clones identified as above were used in a loss of binding screen on LNCaP cells transfected using a genome wide CRISPR knockout library in order to identify functionally related genes required for biomarker expression. Next, RNA-seq experiments are utilized to probe differential gene expression (pathway analysis). Further. mRNA transcript isoforms of the CRISPR identified genes within various culture conditions and methods are identified. In these studies, information provided by the CRISPR knockout library screening and RNA-seq studies can be validated via qRTPCR of mRNAs identified as above and western blots of down-/upregulated gene products can be performed. “Dose” response can be performed to characterize the range and sensitivity of biomarker expression to the correlated culture condition. Also, using RNA interference for knockdown studies will allow for disruption of biochemical pathways to verify relevance of pathways/genes to biomarker expression. In these studies, cell surface binding of the ø clones described above and expression levels of identified mRNA transcripts are investigated upon other human prostate carcinoma cell lines. Further, these cell lines can be grown in various culture conditions and methods, and statistically correlated to 8 measures of cell health. In these studies, investigation into the biomarkers expression within xenografted tumors and human clinical samples is performed. Excised xenografted LNCaP tumor tissues along with human normal prostate, benign growth, primary tumor, and metastatic tumor tissues can be probed for biomarker expression using the ø clones and qPCR. Further, single cell RNA-seq analysis of excised xenografted LNCaP tumor tissue can allow for the study of biomarker mRNA expression levels and pathway analysis within an animal model of human cancer. This, in turn, can be compared to that of 2D cultured LNCaP.

Significance: Issues with Data Reproducibility: In recent years the scientific community has begun a serious conversation about reproducibility of published data. While this issue is expansive and covers researchers of all backgrounds, a significant amount of attention has been focused upon cultured cell lines. A study commissioned by ATCC in 2020 revealed that around 90% of the respondents “would like to see granting agencies and publishers do more to enforce requirements that could improve reproducibility” (1). They disclosed that 65% of respondents felt that the issue of reproducibility is an urgent problem. Interestingly, 87% said that they borrow cell lines, but only 29% said that they re-authenticate cell lines. Scientists report that data generated from vendorsupplied cells resulted in fewer problems with reproducibility. And while, 9 out of 10 scientists continue to borrow cell lines from colleagues, they also report more issues with data replication. These findings are in agreement with similar surveys published by Nature and eLife (2. 3).

Unchecked Cell Line Contamination and Misidentification: A well-documented problem impacting data repeatability is contaminated/misidentified cell lines (CMCL) (4-6). The continued use of CMCL affects scientific research and thus impacts development of medically important drugs (7-10). HeLa contamination was first noted in the 1960's (11, 12). CMCL have continuously been utilized through the decades with a large percentage of these being either contaminated with HeLa or mislabeled (8, 11, 13-15). Recently published papers predict that 20%-30% of all cell lines in use may be CMCL (4, 12, 15, 16). The presence of “false” cell lines has been attributed to lack of awareness, documentation, and access to equipment needed for verification.

Unchecked Variables Adding to the Reproducibility Crisis: A larger and more complex issue is the myriad of experimental variables that have direct impact upon cultured mammalian cell phenotype. In the late 1950s, Harry Eagle and Theodore Puck (17, 18) described the use of fetal bovine serum (FBS) for the attachment and growth of mammalian cells in vitro, as well as other nutritional requirements of mammalian cells (17-22). FBS has since been utilized as the main source of biochemical growth signals and nutrients for tissue culture media. Unfortunately, FBS has a long and very well documented list of problems including bacterial, mycoplasma, viral contamination, wide variability of components, exogenous molecules, and even geographical location of cattle production effecting composition (23-28). To this end the International Serum Industry Association has instituted guidelines for quality control and standardization. These guidelines entail a long list of tests required for Certificate of Analysis (CoA) (29). These tests include at a minimum, sterility, mycoplasma, virus, pH, osmolality, total protein, endotoxin, hemoglobin, immunoglobulin, electrophoretic pattern, and performance testing. Unfortunately, batch to batch variability still exists in serum components and presence of contaminants, such as, antibiotics, hormones, and other drugs utilized by the cattle industry. These differences lead to wide ranging biological activities within the cultured cells and thus leads to experimental variability. This in turn has serious impact upon cell growth, expansion, and performance of cultured cells, which then negatively effects intra/interlaboratory reproducibility (30, 31). It is important to note that the use of FBS is slowly being phased out. Media formulations with FBS are slowly being replaced with defined medias. However, to date, the majority of these defined medias are for use with primary and/or multipotent/pluripotent cells.

In addition to the above-mentioned CMCL and FBS composition variables, methodology also has a great impact upon cell phenotype. It is well documented that passage number, media formulation, and seeding density all effect morphology, proliferation rate, cell density, glucose transporter expression, and brush border enzyme activities of human intestinal Caco-2 cells (32-37). Another example, granulosa cells at low plating density exhibit estrogenic phenotype, and those at high density exhibit luteinization (38). Additional problems of over-culturing are beginning to be addressed within the literature. Culturing cell lines for too long with various unintentional selective pressures results in drift (39-41). This in turn generates a myriad of cell lines with the same name but unpredictable, reduced, and/or altered biology (9, 42). These divergent cell lines exhibit different gene expression leading to different morphology, development, and key functions (41, 43, 44). Thus, reproducible results between laboratories are negatively affected. Additionally, generated data is no longer representative of the original source material (9). For example, high passage number prostate carcinoma cells, LNCaP, respond differently to androgens and retinoids when compared to low passage number (45, 46). Reports of cell lines responding to various/small changes in their environment highlight the plasticity and sensitivity of mammalian cells.

Canonization of False Facts and How to Define Variables: These multiple pitfalls (CMCL, different methodologies, drifting phenotype, and/or divergent evolution) currently prevent the goal of improved reproducibility. Importantly, there is currently no universally accepted way to methodically verify the phenotypic status of cell lines within the common laboratory, thus, the scientific community continues to build upon research performed with CMCL and/or cells with changed phenotype (4, 7, 9, 10, 15). This process has been referred to as “the canonization of false-facts” (3). We propose that, while the reporting of data from CMCL may indeed be “false facts”, it might be that many of the so called “false facts” are not truly false, but instead work performed within an ill-defined set of variables. In other words, the culturing conditions of the cell lines and the resulting phenotype are not well defined. Thus, the problem facing the scientific community is how to define and control the multitude of variables facing researchers using mammalian tissue culture. Additional and equally important questions are within which context should these variables be investigated? And what phenotype should be agreed upon?

Current “-omics” Technologies and Their Limitations: While there is no universal protocol for the definition of cell phenotype, some techniques include use of transcriptomics, proteomics, or metabolomics for the analysis of phenotype (54). While there are academic databases available for the comparative analysis of “-omics” data, to date, there remain issues with the standardization of the data formatting and analysis (54. 55). Another significant issue with the use of “-omics” data is the enormous and unwieldy amount of data generated (56). The increasing complexity of data from thousands of genes, to hundreds of thousands of RNA transcripts, to millions of protein products results in the need for specialized training; both for the generation and for the analysis of the data. All together these issues place the analysis of phenotypic characterization of cell lines outside of the abilities of most laboratories. The cost of equipment and analysis, and the technical requirements, are too heavy of a burden for the common laboratory (56). This is especially true if we wish to develop and encourage frequent, small-scale use within individual laboratories (57). Additionally, these analytical approaches using “-omics” data are limited in the type of biomarkers that are investigable. The definition of cell phenotype should be based upon more than just a single type of molecule (DNA vs RNA vs protein): regardless of how broad the analysis of the macromolecule type might be. In order to truly understand the biochemical processes within cultured cells multiple tiers of biologically relevant molecules must be monitored. And while, theoretically, the use of “-omics” technologies is able to do that, again, the technical hurdles and costs are too large for most laboratories.

Use of Biomarkers Across Multiple Models of Human Biology: Use of biomarkers within the context of translational studies from in vitro to in vivo is a current hot topic (47,48). Recent publications call for improved biomarker identification strategies in order to improve translatability and drug development (49-51). These studies highlight the utility of biomarkers in the framework of biological research. In this light, we believe that in order to modify/optimize the traditional 2D monolayer culture method, we must characterize the basic phenotype of cells grown within traditional 2D culture conditions but also maintain an eye on how the 2D in vitro phenotype differs from in vivo models and/or clinical presentation. In other words, it would be more practical to wait to change 2D culture systems until after relevant information of cellular phenotype and/or biomarker expressions within 2D culture systems has been collected and how these data compare with in vivo models and/or clinical presentation. Only then would it be prudent to alter/optimize the in vitro growth method without adding further confusion and complications to the myriad of undefined variables within the current models of human disease. To this end, we are proposing to collect a wide range of information, including, biomarker/mRNA expression levels—due to various in vitro growth conditions—and how they correlated to basic measures of cell health.

Strategies to Bring Cell Line Characterization to Every Laboratory; Some products have been/are currently being developed specifically for genetic identification. Currently, short tandem repeat (STR) analysis is able to genetically identify cell lines and has been validated by American National Standards Institute and the American Type Culture Collection (ATCC) resulting in a documentary standard (ASN-0002) (5, 6, 58). This technology has successfully focused upon a small subset of genetic sequence for the identification of individuals and/or cell lines: versus analyzing the entire genome. However. STR analysis is unable to identify spontaneous mutations outside of the amplicon or identify cell lines that are genetically identical but phenotypically different. Thus, we've proposed to identify and characterize a small subset of biologically relevant biomarkers and associated mRNA transcripts for the characterization of a set of phenotypes within cultured cells. A cheap and easy to use kit (or mail-in service) able to monitor cultured cells for drifting phenotype can be of great benefit to the scientific community. To this end, we disclose development of a method able to describe aspects of the cell lines' phenotype at the time of an experiment. This method can be developed into a kit that can be provided to the end user directly or may be offered as a mail-in service. This kit can 1) verify phenotypic characteristics of the tissue type and/or specific cell line, 2) verify correct culturing conditions and/or age of cell line, and 3) be able to discern “drifting” cell lines. Here “drift” is defined as biochemical changes resulting from epigenetic, transcriptional and/or translational changes, or changes to post-translational modification or processing. This method targets and quantifies both mRNA and cell surface biomarkers to truly monitor a phenotype profile. In short, to the disclosed methods identify and characterize a subset of biomarkers with relevant mRNA transcripts that have variable expression levels in response to changes in culture conditions and/or age. Results published from all cell lines should have short tandem repeat (STR) analytics and a description of the phenotype (both mRNA and protein biomarker) profiles of the cell line either at the time of the experiment or available from monthly reports (as part of general laboratory maintenance). These data would provide focused information from three tiers of cell biology: DNA (via STR analysis), cell surface biomarkers, and relevant RNA transcripts. This analysis will provide researchers with information for the comparison of data between research groups, and may also explain the differences in results.

The Benefit of Employing the Proposed Kit Service: There exists a real need for a kit/mail-in service able to describe mRNA transcript/biomarker status within cells (e.g., cell lines). This is due specifically to the unfortunate fact that many researchers may be slow to relinquish old frozen stocks, stocks with incomplete historical data, and/or unique cell lines derived from questionable sources. Additionally, there remains within the scientific community a large number of journal articles reporting the use of different media for the same cell line (59-61). Thus, a common, cheap, and easy-to-use kit (or mail-in service) with a long shelf-life, used to determine/verify biomarker expression levels may help change the scientific culture. If a researcher is not amenable to removing questionable cells from their laboratory, or using a different media composition, they may report the biomarker matrix along with experimental results. Additionally, the continued use of FBS and the unintended repercussions upon the cellular phenotype profile resulting from it, can, at minimum, be noted. Additionally, this technology can aid in the development of synthetic media recipes (i.e., FBS free media). This added information can aid the scientific community in evaluating data and/or aid in the determination of the need for further standardization of cell culture methods/media. The information from this kit can be intended to provide a framework from which researchers can compare their specialized data.

### Innovation: Limitations of Current Technologies:

While there are some proposals for the quality control and identification of cell lines, most of them focus on either DNA, RNA, or protein expression profiling, or rely upon characterization of a single biochemical pathway (ex. receptor signaling) (40, 45, 46, 58, 62). These approaches are unable to encompass a broad biochemical profile of a cell line, and thus, are unable to truly monitor a cell line for phenotypic drift. Phenotypic drift could, theoretically, first reveal itself within changes to DNA transcription, due to epigenetic changes (63). But it could also be defined as changes to the cell surface levels and/or localization of biomarkers (i.e., receptor internalization or dimerization) in response to extracellular stimuli (64, 65). Thus, simultaneously monitoring both RNA and cell surface biomarker expression levels in order to properly define a cellular phenotype profile has value. To date, there is no technology able to globally identify and/or quantify changes of carbohydrate antigens. Thus, the state-of-the-art “-omics” technologies currently available are unable to 1) globally monitor multiple tiers of biology in a cost efficient and user-friendly manner, and 2) are not able to assess all forms of cell surface biomarkers.

Closing the Gap by Monitoring Three Tiers of Biology: Disclosed here is a unique protocol able to truly evaluate the phenotypic status of a cell line by quantifying both mRNA and all forms of associated cell surface biomarkers. Disclosed is use filamentous bacteriophage (phage) containing a single stranded DNA (ssDNA) genome, genetically modified to express a unique foreign targeting peptide (66). This targeting peptide can possess specificity and affinity for any type of cell surface target (67). This in turn allows use of the ssDNA of cell surface bound phage as a nucleic tag able to translate protein/lipid/carbohydrate expression into a quantitative polymerase chain reaction (qPCR) based signal. Additionally, this PCR assessment of phage ssDNA can be easily coupled to the analysis of cellular mRNA sequence. This idea is the basis of a method that uses multiple unique phage clones, each expressing a different targeting peptide, thus allowing for quick and efficient investigation of multiple biomarkers along with associated mRNA transcripts. Instead of analyzing the total expression of a specific type of biomarker (RNA/transcriptomics, protein/proteomics, etc.), the disclosed method analyzes as many types of biomarkers as possible (modified protein, carbohydrates, lipids, etc.), but in a targeted and focused manner. Utilization of a matrix database (or scorecard) system containing validated ranges of expression levels for each biomarker/mRNA pair, can enable the identification of drifting or phenotypically different cells.

Questions Answered by the Proposed Method: Simultaneous probing of multiple mRNA/biomarker pairs into a single PCR based system can provide direct evidence of tissue type, cell line identity, as well as be able to provide initial evidence of phenotypic drift. Directly comparing changing mRNA levels to changing cell surface biomarker levels within the same cell line over the course of time can provide additional information about altered biochemical pathways. Use of this proposed product in conjugation with STR analysis can enable scientists to probe three tiers of cell biology. Our proposed techniques will probe more forms of biologically relevant biomarkers (lipids and carbohydrates) in a way that the “-omics” technology are not currently. Importantly, to date, there are no forms of “-omics” able to probe network signaling and/or non-canonical biomarker status. Of equal importance, this proposed method will be simple enough to be utilized within a wide range of cell types and culture conditions/methods. For example, we envision the future kit to be able to compare and contrast biomarker/mRNA status across 2D, 3D, animal model, and human clinical samples.

Details of the Disclosed Kit: The disclosed kit will be low-throughput with a minimum number of phage clones, however, an offered mail-in service can easily be scaled up via robotics and other high-throughput technologies to include increasing numbers and complexities of sentinel biomarker surveillance. A panel of multiple phage clones, each specific for a different biomarker, can be utilized to simultaneously probe the status of multiple cell surface biomarkers. The paired phage clones/biomarkers can be characterized and validated for a known expression level within multiple cell lines of a specific tissue type (LNCaP vs DU145 vs PC3, etc). Furthermore, the levels of expressed mRNA transcript(s) associated with biomarker expression can also be characterized and validated. A matrix of biomarkers/phage clones/mRNA transcripts can allow for sensitive detection of the presence/absence of important biomarkers. Utilization of a matrix database system can identify drifting cells (i.e., changes in biomarker/mRNA levels) due to prolonged culturing, inappropriate media composition, confluency, etc. In this way, one can probe for numerous biomarkers in an array of different cell lines (but same tissue type), in multiple culturing methods, using a single common panel of phage clones. This kit/service can be utilized prior to initiation of important experiments, or possibly to be performed monthly as part of the general laboratory maintenance

A New Process for New Data: The disclosed methods combine techniques in an innovative way. The proposed method can provide an answer to NIH/NIGMS previous requests for “reliable, rapid, cost effective, and easy to use” technologies in order to “facilitate the type of frequent, small scale use prevalent in individual laboratories” (57). And it will accomplish this by providing a “method for distinguishing between cell lines based on phenotype └and┘ signaling network activities” (57). This new process will yield new data, and potentially reveal new mechanisms of cancer systems biology, targets, and understanding in the field of cancer research. This added information will 1) help the standardization of mammalian tissue culture by further defining and aiding in the control of variables, 2) aid in the comparison of data within and across laboratories, and 3) enable the fine tuning of in vitro models of human biology. Finally, the combination of two distinct blinded combinatorial techniques, that of phage display and clustered regularly interspaced short palindromic repeats (CRISPR) knockout libraries, is also innovative. To date, identification of a single phage display-selected clone's binding partner/biomarker requires scrutiny and labor. Utilization of a CRISPR whole genome knockout library has enabled identification of a family of functionally related genes required for the targeted biomarker. This in turn can aid in timely identification of phenotypically relevant mRNA transcripts directly related to the selected clones/biomarkers.

Objective: Disclosed herein is panel of sentinel biomarker targeting phage clones and associated mRNA transcripts as well as (2) characterization/validation of both biomarker cell surface expression and associated mRNA transcript expression levels within various in vitro and ex vivo settings. A commercialized kit can be developed. Sentinel biomarkers can be developed with expression patterns identified across multiple models of human diseases. Furthermore, the data generated from selection and screening protocols can be directly compared to concurrently generated data from multiple assays to describe aspects of the cell health. Thus, phage clones/biomarkers can be selected for characteristic cell binding within a known set of methodology variables and cell health characteristics.

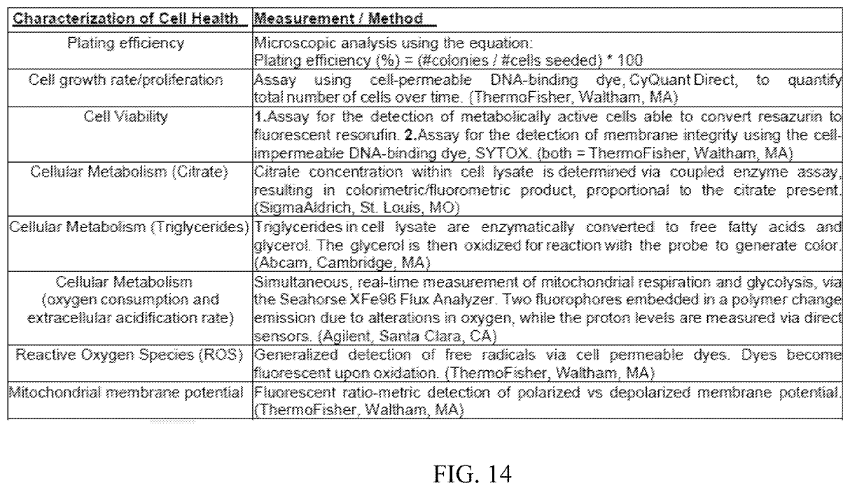

Eight Measures of Cell Health (MCH): Within this proposed project, biomarker expression levels are compared to data generated from 8 assays intended to describe and quantitate aspects of cell health (FIG. 14). For example, oxidative stress, mitochondrial disfunction, and altered metabolism have all been described in various prostate cancer cell lines, including, LNCaP, DU145, and PC3 (68, 69). Changes in these systems can have global impact upon cellular phenotype/gene expression levels (52,53), in part, due to the biochemical crosstalk between oxidative phosphorylation, aerobic glycolysis, lipid metabolism, and hypoxia within prostate cancer cells (69-72). Thus, the proposed 8 MCH can include a generic measurement of cellular metabolism (oxygen consumption and extracellular pH change), as well as cell-specific metabolism of citrate and triglycerides. Mitochondrial membrane potential, ROS production, and the more traditional observations of cell health can also be recorded; plating efficiency, growth rate/proliferation, and viability. These 8 MCH were selected to provide general information about the status of cell health as a consequence of culturing method and/or condition. And the more traditional observations of cell health (plating efficiency, growth rate, and viability) are included in an effort to bridge old data to newer, more specific forms of phenotypic data. Knowledge of the general health of the cells cultured with the various proposed methodologies/conditions disclosed herein and determination of any correlation between the targeted biomarker and MCH, can aid in the understanding of the data generated from the CRISPR and RNASeq protocols disclosed here.

In some embodiments, normal prostate tissue can be unique in its metabolic phenotype: the human prostate gland contains and excretes high levels of citrate, in contrast to most cell types where the mitochondria oxidize citrate via the TCA cycle (73, 74). However, a signature of many cancerous prostate cells, is the use of the TCA cycle for more efficient energy production (75). Consequently, cancerous prostate cells often contain a reduced level of citrate (74). Higgins et al. reported that high passage number LNCaP cells possessed altered gene expression resulting in metabolism conversion from oxidative phosphorylation to glycolysis. And Gao et al. showed that individual prostate cell lines each possessed their own glycolytic features (74). Thus, quantification of citrate can aid in the differentiation of normal vs. cancer prostate phenotypes and to track changes in cellular metabolism over time.

Another hallmark of cultured prostate cancer cell lines is accumulation of fatty acids. Sorvina et al. described complex lipid profiles for cancerous, benign, and normal prostate cell lines (69). These studies highlight the need of both endogenous and exogenous sources of lipids for cancerous prostate cells (69, 76). However, a simplified measure of de novo lipogenesis is accumulation of triglycerides. Additionally, lipid droplets are common in cells under stress (77, 78). Thus, quantification of triglycerides can be analyzed both to differentiate between normal and cancer lines and to characterize cellular responses to stress.

Mitochondria are essential for multiple cellular functions including metabolism, cellular respiration, calcium storage, apoptosis regulation, etc (79, 80). Normal cellular/mitochondrial activities require a balanced redox state. However, when there is an imbalance in this state, oxidative stress occurs, which in turn, contributes to cytotoxicity (81). Thus, quantification of the mitochondrial membrane potential as a measure of mitochondrial health is included in the seven MCH. Mitochondrial health status can aid in the characterization of cell age/passage number and appropriate/inappropriate culture conditions (82).

Relative oxygen species (ROS) are generated via many different biochemical pathways. Importantly, excess ROS are coupled with oxidative stress and are a marker of aggressiveness in prostate cancer (70, 71). Kumar et. al. revealed increased ROS production within PC3, DU145, and LNCaP cell lines and were able to show that the ROS were produced via both mitochondria and NAD(P)H oxidase (Nox) systems (70). Appropriate levels of ROS within cells are required to maintain redox balance and proper cellular signaling (83). Additionally, increased ROS production is proportional to aggressive phenotype. Thus, ROS is measured/quantified as a part of the eight MCH.

Data: Use of multiple phage display selections to overcome the inefficiency of combinatorial selection against a complex selector: The main principle of phage display technique is the use of multiple iterations of phage binding/selection, washing, and subsequent amplification. This process reduces the number of unique peptide sequences, amplifies the number/concentration of each clone, and thus generates greater same extent when selecting against a complex target (i.e., a mammalian cell surface). Consequently, we performed a comparative selection technique: in which the sequence output of multiple selections were compared to each other in order to identify peptide targeting sequences specific to the intended target (FIGS. 3A-3F). More specifically, we performed such a comparative phage display selection against LNCaP (+ cell line) and HEK-293 (− control cell line). Further, to maximize the potential of these phage display selections we utilized different elution techniques to probe for high abundance biomarkers (direct phage display selection), as well as low abundance biomarkers (depletion phage display selection). From this, a large number of phage clones unique to the elution protocol, as well as the depletion protocol were found (FIGS. 3A & 3C). Many of these were found binding to low passage number cells and not high passage number cells (and vice versa) (FIG. 3E). It was equally important to discern whether or not these phage clones are unique to the LNCaP cell line. Thus, the parallel selections on the control cell line. HEK293 (FIGS. 3B & 3D) were performed. All of this work was performed upon cells at passage number 6 or less (˜20 days of continuous culture) (Selection #1). This work was then repeated using cultured cells at passage number ˜40 (˜6 months of continuous culture) (Selection #2) (FIGS. 3C-3E). The combined number of identified unique phage clones=127,662.

Development and optimization of phage quantification method for use in the screening of selected phage clones: We developed a protocol for the quantification of phage bound to cell surface using qPCR with a significant increase in both range and greater sensitivity of detection over previously utilized protocols. We first developed a repeatable quantitative standard curve with a range of detection of 10³ to 10¹² v/mL. This protocol has a limit of detection (LOD) of 10³ v/mL and a limit of quantification (LOQ) of 10⁴ v/mL. In comparison, a cell-based ELISA protocol utilizing biotinylated phage has a lower limit of ˜10⁵-10⁶ v/mL. While a cell binding assay using phage titer for quantification has a lower limit of ˜10⁴-10⁵ TU/mL. It is important to keep in mind that only about 5-10% of fUSE5 phage clones are infectious, thus this lower limit is equivalent to ˜10⁶-10⁷ v/mL. Moreover, recreation of selected phage clones in a novel vector, f3TR-1, allowed for trypsin elution and further optimizes quantification of phage by avoiding potential bias in elution (detergent vs acid) (84, 107).

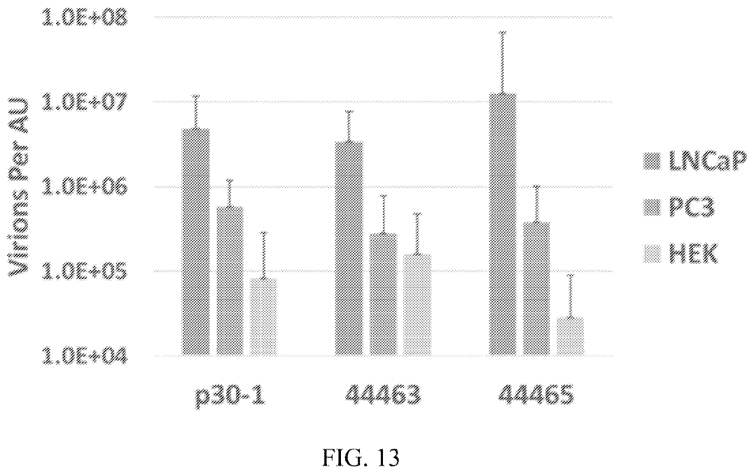

Screening of selected phage clones. In total, as a proof of principle, 40 phage clones were recreated and screened for saturable binding to LNCaP. Of these 40, 11 were found to possess saturable binding. In short, using four parameter logistic regression to analyze the binding data, we found that the maximum binding values (aka. Bmax) to range from 5.42×10⁹ to 2.69×10¹¹ v/mL. These values are equivalent to 9.02 pM to 446.95 pM of phage. The calculated EC₅₀ is equivalent to KD because the binding is performed upon fixed cells. Thus, there is no “response” and the subsequent relationship between the receptor occupancy and response is linear. These KD values ranged from 4.83 pM to 14.30 pM. Of note, the f3TR-1 phage display 5 copies of targeting peptide, thus apparent KD values of multivalent particles are improved by the avidity effect. Cell binding assays with the 40 clones revealed that 27 phage clones did not have saturable binding and thus did not result in Bmax or KD. And 9 clones did not bind LNCaP cells with a high enough preferential binding. Cell line specificity is defined as the ratio of HEK293:LNCaP EC50 values. In comparison, the data from cell binding assays for 4 clones (44467, 44465, 44463, and 3) did result in a ratio of HEK:LNCaP binding of 0.009 or less. Next, these 4 clones were further screened against LNCaP and HEK293 cells at four different passage numbers (10, 20, 30, and 40). Clones 44465 and 44463 were then identified as targeting biomarkers with LNCaP expression that reduces over time (FIG. 7). Further, we screened these clones for changes in cell surface binding due to changes in confluency. Clones 44463, 44465, and 44467 were then identified as sensitive to changes in confluency (FIG. 8). And finally, we screened the 44463 and 44465 clones for biomarker expression levels on another human prostate cell line, PC3 (FIG. 13). Preliminary testing of phage binding to multiple cell lines suggest that each cell line has a unique biomarker expression level. These data agree with previous phage display selection publications. For example, data exist showing KCCYSL phage clone (identified from phage display selections against purified antigen) and the previously selected phage clones. H5 and G1. (selected against whole PC-3 human prostate carcinoma cells) (108, 109, 111). These results highlight the ability of this type of protocol to isolate peptides/biomarkers that range from a propensity of carcinoma cell binding to binding only prostate carcinoma cell lines. Importantly, the levels of phage binding (fluorescence intensity) is unique for each cell type.

Loss of binding assay performed on LNCaP cells transfected with CRISPR knockout library with whole genome coverage: First, positive (+) control (p30-1), negative (−) control (f88), 44463, and 44465 phage clones were fluorescently labeled with AF488. Next, LNCaP cells at passage number 4 were first stably transfected with Cas9 with antibiotic selection. Then transfected with a whole genome knockout library of CRISPR/gRNA constructs, again with antibiotic selection and RFP internal positive control. Finally, successful transfection (RFP expression) was verified, and changes in phage cell binding quantified using cell flow cytometry. Next, LNCaP cells with no phage binding or reduced phage binding were sorted out of the CRISPR knockout library population (sorted out and collected RFP+/AF488-cell population). The gDNA of these individual cell populations were then isolated, purified, and used in nested PCR to amplify gRNA identification sequences and then add Illuminia sequencing adaptors with index codes. Finally, NGS data was analyzed at the Bioinformatics and Analytics Core (BAC) where they organized the gRNA/gene ID by counts.

The positive control, p30-1 clone, was selected and affinity maturated against purified Thomsen-Friendreich (TF) Antigen (105), a disaccharide carbohydrate tumor antigen (85-88). It is a Core-1 (Galβ1-3GalNAcα-1Thr/Ser) structure of O-glycans (88). The TF antigen is a confirmed tumor antigen, though the exact pathway of synthesis of it is unknown, it is generally thought that TF antigen is usually hidden within longer carbohydrate chains on normal tissues (89). The cell population analyzed for the loss of binding to the p30-1 clone revealed 2 of the 6 known mucin proteins within prostate. MUC3A and MUC4 (FIG. 9) (90). Both are membrane bound and MUC4 is known to display TF (91). Interestingly, there were 4 different β-1,3-galactosyltransferases (B3GALT1, -2, -4, and -5). These enzymes transfer galactose from UDP-galactose to substrates with a terminal β-GlcNAc residue; required for the first step after the Core-1 formation (85, 87). From there the Core-1 O-glycosylation biochemical pathways exist in equilibrium between sulfotransferases, sialyltransferases, and Fuc-transferases. While not wishing to be bound by theory, we hypothesize that the decrease in p30-1 binding on cells with ST3GAL4, ST6GAL2, FUT2, and FUT8, knockouts would be due to further perturbations within the O-glycosylation equilibrium potentially pushing glycosylation towards Core-2 (Core-1 with branched B1-6GlcNAc additions) (85). Importantly, the C1GALT1C1 is a molecular chaperone with known influence on the synthesis of TF antigen (92).

Analysis of clone 44463 loss of binding data revealed a cluster of functionally related genes in the semaphorin, plexin, ephrin/eph receptor neuronal guidance system/axon outgrowth pathways (FIG. 10). However, this system has recently been linked to cancer progression, angiogenesis, and metastasis (93, 94). The string database reports 7 experimentally determined interactions (out of the 12 total genes) between EPHA8 (Ephrin type-A receptor 8) and various ephrins, semaphorins, and plexins (pink lines in FIG. 7). Suggesting that EPHA8 maybe the center of this cluster. These pathways are known to influence/control cytoskeleton remodeling through the GTPases Rho and Rac (95-98).

Analysis of clone 44465 data revealed 2 related clusters (FIG. 11). The larger cluster contains 10 genes (FIG. 11). Nine were confirmed for expression within peroxisomes and the last (PPARA=peroxisome proliferator activated receptor alpha) is a ligand-activated transcription factor and key regulator of lipid metabolism (99, 100). 4 genes are members of the peroxisomal biogenesis factor (PEX) family: specific for peroxisome proliferation and movement (101). While the ACOX1/2, SCP2, ACAA1, and the HSD17B4 genes are all involved in metabolism of fatty acids/lipids (102-104). The smaller cluster contains genes required for phagocytosis (green circles), genes associated with membrane rafts (purple circles), and genes associated with vesicle membranes (red circles) (FIG. 11). We, thus, hypothesize that 44465 binds to a type of lipid.

Approach: We can screen more clones by comparing binding within appropriate versus inappropriate culture methods. The clones of interest/biomarkers of interest can then be further characterized within mouse model of cancer and clinical samples; each of which can be normalized via cell number. The cell numbers of complex samples can be determined microscopically via Celleste Image Analysis software. To minimize variables the same source material, LNCaP clone FGC, can be used throughout. All screenings and binding characterizations can be performed against formalin fixed cells/tissues; to minimize artifact from handling and enable future work for mailin service.

Screening of Selected Phages. We can continue the data mining of selected sequences by recreating additional clones for screening of saturable binding on low passage number fixed LNCaP cells. A high throughput cell binding, trypsin elution, qPCR quantification can be used to identify individual phage with saturable binding (defined by Bmax and KD). Utilization of (+/−) clones as internal controls within each plate can ensure consistent quality of data. The (+) control clone is, p30-1, which binds TF Antigen (112). (−) control phage clone, f88, contains no foreign peptide on coat protein III (106). Each phage clone can be tested with at least 10 replicates, and each plate of cells can also contain a positive and negative control phage clone. Thus, we can analyze 24 96-well plates. Additional clones with K_{D} values of 100 pM or less will be carried forward to the second stage of screening.

The 2nd stage screening protocols can be performed using 5 different ages of LNCaP cells (passage numbers 5, 15, 25, 35, and 45) (114) and inappropriate culture conditions including CO₂ at 3% and 7%, different lots of FBS, different media recipes, and difference in confluency (10%, 50%, and 100% confluency). Here we can use confluency in place of cell number. This decision is made to account for differences in cell size and is intended to focus instead upon cell-cell contacts. A short series of experiments can be used to determine cell number needed for desired cell confluence.) In short, using a single concentration of phage (K_{D} value) within the qPCR assay, the binding levels of each phage clone to cells grown within the 13 different variables/growth conditions (1 normal and 12 abnormal) can be compared. A stepwise regression analysis can be employed to detect conditions/variables that result in significantly different phage binding. Additionally, stepwise regression analysis can allow observations of the magnitude of contribution of each variable. This can require 40 replicates to identify the 90% confidence interval. Finally, the 8 MCH can be investigated within cells grown in the same set of variables. Correlation between the phage binding levels and the 8 MCH to the 13 different growth conditions/variables can be investigated. We can determine the correlation coefficients to parse the potential relationships between two or more variables.

Outcomes: A database with phage K_{D} and Bₘₐₓ values for phage clones with associated correlation coefficients for 8 MCH and/or 13 different culture conditions and methods can result.

CRISPR Knockout Library to Identify Gene(s) Required for Biomarker Expression. The clones of interest as described above are utilized in a loss of binding screen on LNCaP cells transfected with human whole-genome knockout CRISPR library (Cellecta, Mountain View, CA). LNCaP cells stably transfected with Cas9 are transduced with the knockout library. Phage fluorescently labeled with NHS-AlexaFluor488 can be utilized in a cell sorting flow cytometry protocol on a Beckman Coulter MoFlo XDP (Cell and Immunobiology Core at MU) to collect cells with little to no phage binding. Finally, sgRNA targeted genes within the selected LNCaP-Cas9 knockout library subpopulations can be identified by isolating genomic DNA and PCR amplifying the construct. The resulting PCR product can be submitted for ligation of the necessary Illumina adaptor sequences for NGS. The 10 pooled samples can be read at a depth of ˜260 million paired end reads. DNA sequence will be submitted for trimming, analysis, and gene identification. Using Feature Annotation Using Nonnegative matrix factorization and GeneMesh web-based software we will be able to determine the functional relationships amongst the identified genes.

Utilization of RNA-seq for the Analysis of the mRNA Products from the Identified Functionally Related Genes. Differential gene expression and transcript isoform studies can be performed via whole transcriptome analysis. Cells grown in “normal” conditions will be compared to cells grown in “abnormal” conditions. From this, changes in mRNA transcript levels or isoform expressions can be identified. “Normal” conditions will be defined as low passage number LNCaP cells grown as proscribed by ATCC. Total RNA can be collected from LNCaP cells using a Total RNA Miniprep Kit (New England Biolabs). The mRNA can be prepared for reverse transcription. The resulting cDNA can be purified and amplified using PCR primers with Illuminia index codes. At this point, the indexed cDNA library samples will be submitted to the GTC where the quality of the libraries assessed, pooled, and sequenced at a depth of ˜50 million reads per sample. Finally, for the data output, the sequences will be aligned and mapped using STAR alignment The phages and mRNA transcripts will be utilized within the qPCR/qRT-PCR methodology.

Pathway Analyses from the Differential Gene Expression Study. The same RNA-seq data from above can be further probed for pathway analysis. Gene-gene associations can be analyzed for differential gene expressions or differentially connected gene modules (121). KEGG pathways can be incorporated into our differential expression analyses (122). In short, the data can be imported into the BAC pipeline, annotated, packaged by gage (Generally Applicable Gene-set Enrichment for Pathway Analysis), and analyzed within the KEGG pathways package.

Alternative methods: It is possible to use an arrayed lentivirus library and screen individual knockouts. It is possible that the screening might need to be performed using cells grown in specific conditions, such as high passage number, specific [CO2], confluency, etc. Further, individual genes can be targeted via siRNA to verify CRISPR results and probe transcript isoforms. Validation of the Information Gathered from CRISPR and Transcriptomics. In short, we will further investigate each of the phage clones and associated mRNA transcripts described above. First, the cells are grown within the same set of defined variables as required as described above, both normal and abnormal. Next, the cellular responses to these culture conditions, as defined by the differential gene expression study can be probed using qRT-PCR of mRNA transcripts and then compared to the expression levels of the chosen diagnostic mRNA transcripts. Further, we can investigate/optimize the selection of housekeeping gene for the normalization of qRT-PCR data, to date, we have used the hypoxanthine-guanine phosphorribosyltransferase HRPT gene. Finally, the expressions of these transcripts can be probed via western blots and qPCR of cell surface bound phage. In this way, we can verify biochemical pathway responses to environmental stimuli and consequent changes to the biomarker expression levels. “Dose” Response. Next, we can investigate the range and sensitivity of responsiveness of the individual biomarkers/mRNA to the correlated variable identified as described above. For example, if the identified biomarker/mRNA expression level is sensitive to CO₂ levels, then we can further investigate this response by growing cells in more comprehensive series of different CO₂ levels (3%, 4%, 5%, 6%, 7%, etc). Or if it is in response to confluency, then we can investigate response by growing cells in a more comprehensive series of different cell confluences (10%, 20%, 30%, 40%, 50%, 60%, etc). Each sample of cells/data point can be analyzed by qRT-PCR of gene transcripts and qPCR of cell surface bound phage. This can again require 40 replicates of each data point to identify the 90% confidence interval. Further, we can also verify the same biochemical responses and probe the strength of these responses within each of the data points within the comprehensive series via western blot and/or more in-depth qRT-PCR. Further, if previous work demonstrated statistically significant correlations between one of the 8 MCH and biomarker expression, then we can verify that the previously observed correlation coefficient is consistent across the comprehensive series. Knockdown Studies. Using RNA interference (RNAi) for knockdown studies we can both verify reduction/elimination of targeted biomarker as well as investigate effects of the inhibition of specific genes. The test system can first be optimized using commercially available positive control. GAPDH, and negative control short interfering RNAs (siRNAs) with the Silencer Cell Ready Transfection Optimization Kit (Thermo Scientific/Invitrogen). This kit uses a lipid carrier for the transfection of cells with siRNA. Thus, we can test a range of lipid carrier concentrations and siRNA concentrations in order to optimize the transfection of LNCaP cells. Gene knockdown can be verified at 24 hours post transfection via qRT-PCR. Once experimental conditions are determined, each of the mRNA transcripts/genes can be selectively knocked down. Again, 24 hours post transfection gene knockdown can be verified via qRT-PCR for the mRNA and qPCR for the biomarker expression levels. Finally, we can investigate the effect of the gene knockdown upon the 8 MCH. In short, siRNA can be used to disrupt biochemical pathways in order to verify relevance to biomarker expression and to investigate potential biochemical crosstalk/links between the previously observed upregulated pathways. In this way we can strengthen the observed and quantified correlations between the biomarker expression level and cellular response to external stimuli Alternative Methods: If siRNA studies are not successful due to off target activities or other issues, then we can utilize small molecule inhibitors to inhibit biochemical pathways and parse relationships between biomarker expression and observed cellular responses to environmental stimuli. Characterization of Phage Clones and associated mRNA Transcripts within the Panel of Prostate Cell Lines. Characterization of the phage clones can be expanded to include human prostate carcinoma cell lines PC3, DU145, MDA-PCa-2b, CA-HPV-10, VCaP, PZ-HPV-7, CA-2B, and NCI-H660. Benign papilloma cell lines, WPE-stem and WPE-int can be used. Normal prostate cell lines, RWPE-1, RWPE-2, PWR-1E, WPE1-NA22, WPE1-NB26, WPE1-NB14, WPE1-NB11, 22Rv1, and WPMY-1 can also be used. Saturation binding experiments utilizing experimental phage clones, positive control clone, and negative control clone, and our qPCR method will be performed. The resulting apparent KD and Bmax values for the new cell lines can then be compared to the values from low passage number LNCaP cultures using two-way ANOVA analyses. Additionally, the list of mRNA transcripts identified within Aim 2 can be interrogated within the same 19 human prostate cell lines again utilizing qRT-PCR.

Quantification of Expression Changes of both Biomarker and mRNA Transcripts Due to Changes in Growth Conditions. The characterization of phage binding to the targeted biomarker and mRNA expression levels within the 19 prostate cell lines can then be expanded to the investigation of changes due to inappropriate growth conditions within this extended list of prostate cell lines. To include cells grown in inappropriate culture conditions: CO2 at 3% and 7%, different lots of FBS, different media recipes, and difference in confluency (10%, 50%, and 100% confluency). Again, the apparent K_{D} value of each phage clone, respectively, can be utilized in these cell binding experiments. Characterization of Selected Phage Clones Binding to Xenografted LNCaP Tumor Tissues and Clinical Samples of Human Tissues. First, the qPCR protocol can be modified for use upon slides of FFPE (formalin-fixed, paraffin-embedded) tissue and the microscopy-based cell number quantification method established. This can be done using slices of FFPE xenografted LNCaP tumor tissues from different tumor bearing mice. The FFPE tissues can be deparaffinized, rehydrated, and blocked. The clonal phage populations at the respective K_{D} values will be incubated with the tissue, washed, and trypsin eluted. Finally, the eluate can then be submitted to qPCR phage quantification. Next, clinical samples of human FFPE can be utilized. Foundational work performed on LNCaP (a cell line derived from lymphatic metastasis of prostate carcinoma) and the other 19 human prostate cell lines, both normal and cancerous, can require the probing of different clinical tissues. Thus, FFPE tissue samples of metastatic prostate carcinoma, primary prostate tumor, benign growth, and normal prostate tissues can be probed. In total, the binding of phage clones to samples of five individuals for each tissue type can be characterized. The data analysis will be limited to comparison of the differences in means of biomarker expression levels between the xenograft, clinical samples, and 2D TC (all normalized by cell number) via two-way ANOVA analyses.

Single Cell Analysis of Excised LNCaP Tumor Tissue. We can excise LNCaP tumor tissue from individual tumor bearing mice and dissociate the tumor cells using a tumor dissociation kit (Miltenyi Biotec, Auburn, CA). In short, excised tumor tissue can be rinsed, minced, and incubated with Miltenyi's proprietary enzyme mix, followed by gentle washing and filtering away of debris. And finally, treatment of cell suspension with red blood cell lysis kit (Miltenyi Biotec) and MACS dead cell removal kit (Miltenyi Biotec). The final populations of tumor cell suspension can then each be divided into two tubes. One tube can be for incubation with fluorescently labeled phage for the cell flow analysis of phage binding, while the other tube is submitted to the DNA core for single cell RNA seq (10× Genomics, San Francisco, CA). A control sample of traditionally cultured 2D LNCaP cells can also be prepared and analyzed via single cell RNA seq. Single cell RNA seq analysis can be performed using at least 10,000 cells per sample with a sequencing depth of 50,000 reads per cell. We can first perform pathway analysis comparisons (as described above) between xenografted tumor cells and cultured cells with an eye on changes within the genes and pathways observed. Next, comparison of fluorescently labeled phage cell surface binding between cultured cells and xenografted tumor cells can be quantified via cell flow cytometry.

Alternative methods. Hydroxyapatite columns may be used to concentrate phage phage eluted from the FFPE slides (113). Or the samples might be concentrated via speedvac centrifuge.

Additional work: We can begin an investigation into precision and repeatability by initiating preliminary intra- and inter-laboratory testing. We can utilize ASTM Standard E-691 (a standard practice for evaluating precision) to develop the necessary statistical techniques needed to analyze inter- and intra-laboratory precision. This method once fully developed will be easily applied to other cell line types, tissue types, and/or panels. Another application of this method can be aiding in the effort to further standardize TC methods and/or media formulation (specifically with no FBS).

### BIBLIOGRAPHY

1. Goldsborough M. Scientific Responsibility in a Time of Crisis. In: ATCC Co, editor. GenomeWeb.com2020.
2. Baker M. 1,500 scientists lift the lid on reproducibility. Nature. 2016; 533 (7604): 452-4. Epub 2016 May 27. doi: 10.1038/533452a. PubMed PMID: 27225100.
3. Nissen S B, Magidson T, Gross K, Bergstrom C T. Publication bias and the canonization of false facts. Elife. 2016; 5. Epub 2016 Dec. 21. doi: 10.7554/eLife.21451. PubMed PMID: 27995896: PMCID: PMC5173326.
4. Horbach S, Halffman W. The ghosts of HeLa: How cell line misidentification contaminates the scientific literature. PLOS One. 2017; 12 (10): e0186281. Epub 2017 Oct. 13. doi: 10.1371/journal.pone.0186281. PubMed PMID: 29023500; PMCID: PMC5638414.
5. American Type Culture Collection Standards Development Organization Workgroup ASN. Cell line misidentification: the beginning of the end. Nat Rev Cancer. 2010; 10 (6): 441-8. Epub 2010 May 8. doi: 10.1038/nrc2852. PubMed PMID: 20448633.
6. Identity crisis. Nature. 2009; 457 (7232): 935-6. Epub 2009 Feb. 20. doi: 10.1038/457935b. PubMed PMID: 19225471.
7. Freedman L P. Know Thy Cells: Improving Biomedical Research Reproducibility. Sci Transl Med. 2015; 7 (294): 294ed7. Epub 2015 Jul. 3. doi: 10.1126/scitranslmed.aac7112. PubMed PMID: 26136474.
8. Vaughan L, Glanzel W, Korch C. Capes-Davis A. Widespread Use of Misidentified Cell Line KB (HeLa): Incorrect Attribution and Its Impact Revealed through Mining the Scientific Literature. Cancer Res. 2017; 77 (11): 2784-8. Epub 2017 Apr. 30. doi: 10.1158/0008-5472.CAN-16-2258. PubMed PMID: 28455420.
9. Hughes P, Marshall D, Reid Y, Parkes H, Gelber C. The costs of using unauthenticated, over-passaged cell lines: how much more data do we need? Biotechniques. 2007; 43 (5): 575, 7-8, 81-2 passim. Epub 2007 Dec. 13. doi: 10.2144/000112598. PubMed PMID: 18072586.
10. Freedman L P, Cockburn I M, Simcoe T S. The Economics of Reproducibility in Preclinical Research. PLOS Biol. 2015; 13 (6): e1002165. Epub 2015 Jun. 10. doi: 10.1371/journal.pbio.1002165. PubMed PMID: 26057340; PMCID: PMC4461318.
11. Gartler S M. Apparent Hela cell contamination of human heteroploid cell lines. Nature. 1968; 217 (5130): 750-1. Epub 1968 Feb. 24. doi: 10.1038/217750a0. PubMed PMID: 5641128.
12. Lavappa K S, Macy M L, Shannon J E. Examination of ATCC stocks for HeLa marker chromosomes in human cell lines. Nature. 1976; 259 (5540): 211-3. Epub 1976 Jan. 22. doi: 10.1038/259211a0. PubMed PMID: 1250349.
13. Stacey G N. Cell culture contamination. Methods Mol Biol. 2011; 731:79-91. Epub 2011 Apr. 26. doi: 10.1007/978-1-61779-080-5_7. PubMed PMID: 21516399.
14. Nardone R M. Curbing rampant cross-contamination and misidentification of cell lines. Biotechniques. 2008; 45 (3): 221-7. Epub 2008 Sep. 27. doi: 10.2144/000112925. PubMed PMID: 18816888.
15. Lacroix M. Persistent use of “false” cell lines. Int J Cancer. 2008; 122 (1): 1-4. Epub 2007 Oct. 26. doi: 10.1002/ijc.23233. PubMed PMID: 17960586.
16. Capes-Davis A, Theodosopoulos G, Atkin I, Drexler H G, Kohara A, MacLeod R A, Masters J R, Nakamura Y, Reid Y A, Reddel R R, Freshney R I, Check your cultures! A list of cross-contaminated or misidentified cell lines. Int J Cancer. 2010; 127 (1): 1-8. Epub 2010 Feb. 10. doi: 10.1002/ijc.25242. PubMed PMID: 20143388.
17. Eagle H. Nutrition needs of mammalian cells in tissue culture. Science. 1955; 122 (3168): 501-14. Epub 1955 Sep. 16. doi: 10.1126/science.122.3168.501. PubMed PMID: 13255879.
18. Puck T T. Quantitative colonial growth of single mammalian cells. Ann NY Acad Sci. 1958; 71 (6): 1141-2. Epub 1958 Sep. 30. doi: 10.1111/j.1749-6632.1958.tb46829.x. PubMed PMID: 13583877.
19. Eagle H, Oyama V I, Levy M. Amino acid requirements of normal and malignant human cells in tissue culture. Arch Biochem Biophys. 1957; 67 (2): 432-46. Epub 1957 Apr. 1. PubMed PMID: 13425639.
20. Eagle H, Oyama V I, Levy M. Freeman A E, Myo-Inositol as an essential growth factor for normal and malignant human cells in tissue culture. J Biol Chem. 1957; 226 (1): 191-205. Epub 1957 May 1. PubMed PMID: 13428752.
21. Fisher H W, Puck T T, Sato G. Molecular Growth Requirements of Single Mammalian Cells: The Action of Fetuin in Promoting Cell Attachment to Glass. Proc Natl Acad Sci USA. 1958; 44 (1): 4-10. Epub 1958 Jan. 1. doi: 10.1073/pnas.44.1.4. PubMed PMID: 16590146; PMCID: PMC335353.
22. Levintow L, Eagle H, Piez K A. The role of glutamine in protein biosynthesis in tissue culture. J Biol Chem. 1957; 227 (2): 929-41. Epub 1957 Aug. 1. PubMed PMID: 13463014.
23. Baker M. Reproducibility: Respect your cells! Nature. 2016; 537 (7620): 433-5. Epub 2016 Sep. 16. doi: 10.1038/537433a. PubMed PMID: 27629646.
24. Groothuis F A, Heringa M B, Nicol B, Hermens J L, Blaauboer B J, Kramer N I. Dose metric considerations in in vitro assays to improve quantitative in vitro-in vivo dose extrapolations. Toxicology. 2015; 332:30-40. Epub 2013 Aug. 28. doi: 10.1016/j.tox.2013.08.012. PubMed PMID: 23978460.
25. Kramer N I, Hermens J L, Schirmer K. The influence of modes of action and physicochemical properties of chemicals on the correlation between in vitro and acute fish toxicity data. Toxicol In Vitro. 2009; 23 (7): 1372-9. Epub 2009 Aug. 5. doi: 10.1016/j.tiv.2009.07.029. PubMed PMID: 19651202.
26. Dormont D. Transmissible spongiform encephalopathy agents and animal sera. Dev Biol Stand. 1999; 99:25-34. Epub 1999 Jul. 15. PubMed PMID: 10404872.
27. Wessman S J. Levings R L. Benefits and risks due to animal serum used in cell culture production. Dev Biol Stand. 1999:99:3-8. Epub 1999 Jul. 15. PubMed PMID: 10404869.
28. Seigel W, Foster L. Fetal Bovine Serum: The Impact of Geography. BioProcessing Journal. 2013; 12 (3).
29. Davis D, Drake Hirschi S. Fetal bovine serum: what you should ask your supplier and why. BioProcessing Journal. 2014; 13 (1).
30. Usta S N, Scharer C D, Xu J, Frey T K, Nash R J. Chemically defined serum-free and xeno-free media for multiple cell lineages. Ann Transl Med. 2014; 2 (10): 97. Epub 2014 Nov. 19. doi: 10.3978/j.issn.2305-5839.2014.09.05. PubMed PMID: 25405151: PMCID: PMC4205861.
31. van der Valk J, Bieback K, Buta C, Cochrane B, Dirks W G, Fu J, Hickman J J, Hohensee C, Kolar R, Liebsch M, Pistollato F, Schulz M, Thieme D, Weber T, Wiest J, Winkler S, Gstraunthaler G. Fetal Bovine Serum (FBS): Past-Present-Future. ALTEX. 2018; 35 (1): 99-118. Epub 2017 Aug. 12. doi: 10.14573/altex.1705101. PubMed PMID: 28800376.
32. Kirikae T, Tamura H, Hashizume M, Kirikae F, Uemura Y, Tanaka S, Yokochi T, Nakano M. Endotoxin contamination in fetal bovine serum and its influence on tumor necrosis factor production by macrophage-like cells J774.1 cultured in the presence of the serum. Int J Immunopharmacol. 1997; 19 (5): 255-62. Epub 1997 May 1. PubMed PMID: 9439764.
33. Smith D R, Brockmann H J, Beekey M A, King T L, Millard M J, Zaldivar-Rae J. Conservation status of the American horseshoe crab, (Limulus polyphemus): a regional assessment. Reviews in Fish Biology and Fisheries. 2017; 27:1573-5184.
34. Merten O W. Virus contaminations of cell cultures-A biotechnological view. Cytotechnology. 2002; 39 (2): 91-116. Epub 2008 Nov. 13. doi: 10.1023/A:1022969101804. PubMed PMID: 19003296: PMCID: PMC3463984.
35. Triglia R P, Linscott W D. Titers of nine complement components, conglutinin and C3b-inactivator in adult and fetal bovine sera. Mol Immunol. 1980; 17 (6): 741-8. Epub 1980 Jun. 1. PubMed PMID: 7432351.
36. Erickson G A, Bolin S R, Landgraf J G. Viral contamination of fetal bovine serum used for tissue culture: risks and concerns. Dev Biol Stand. 1991; 75:173-5. Epub 1991 Jan. 1. PubMed PMID: 1665460.
37. Jennings A. Detecting viruses in sera: methods used and their merits. Dev Biol Stand. 1999; 99:51-9. Epub 1999 Jul. 15. PubMed PMID: 10404876.
38. Portela V M, Zamberlam G, Price C A. Cell plating density alters the ratio of estrogenic to progestagenic enzyme gene expression in cultured granulosa cells. Fertil Steril. 2010; 93 (6): 2050-5. Epub 2009 Mar. 28. doi: 10.1016/j.fertnstert.2009.01.151. PubMed PMID: 19324349.
39. Freedman L P, Gibson M C, Ethier S P, Soule H R, Neve R M, Reid Y A. Reproducibility: changing the policies and culture of cell line authentication. Nat Methods. 2015; 12 (6): 493-7. Epub 2015 May 29. doi: 10.1038/nmeth.3403. PubMed PMID: 26020501.
40. Masters J R, Stacey G N. Changing medium and passaging cell lines. Nat Protoc. 2007; 2 (9): 2276-84. Epub 2007 Sep. 15. doi: 10.1038/nprot.2007.319. PubMed PMID: 17853884.
41. Mathieson W, Kirkland S, Leonard R, Thomas G A. Antimicrobials and in vitro systems: antibiotics and antimycotics alter the proteome of MCF-7 cells in culture. J Cell Biochem. 2011; 112 (8): 2170-8. Epub 2011 Apr. 12. doi: 10.1002/jcb.23143. PubMed PMID: 21480367.
42. Briske-Anderson M J, Finley J W, Newman S M. The influence of culture time and passage number on the morphological and physiological development of Caco-2 cells 1997; 214 (3): 248-57. Epub 1997 Mar. 1. doi: 10.3181/00379727-214-44093. PubMed PMID: 9083258.
43. Chang-Liu C M, Woloschak G E. Effect of passage number on cellular response to DNA-damaging agents: cell survival and gene expression. Cancer Lett. 1997; 113 (1-2): 77-86. Epub 1997 Feb. 26. doi: 10.1016/s0304-3835 (97) 04599-0. PubMed PMID: 9065805.
44. Wenger S L, Senft J R, Sargent L M, Bamezai R, Bairwa N, Grant S G. Comparison of established cell lines at different passages by karyotype and comparative genomic hybridization. Biosci Rep. 2004; 24 (6): 631-9. Epub 2005 Sep. 15. doi: 10.1007/s10540-005-2797-5. PubMed PMID: 16158200.
45. Esquenet M, Swinnen J V, Heyns W, Verhoeven G. LNCaP prostatic adenocarcinoma cells derived from low and high passage numbers display divergent responses not only to androgens but also to retinoids. J Steroid Biochem Mol Biol. 1997; 62 (5-6): 391-9. Epub 1997 Aug. 1. doi: 10.1016/s0960-0760 (97) 00054-x. PubMed PMID: 9449242.
46. Langeler E G, van Uffelen C J, Blankenstein M A, van Steenbrugge G J, Mulder E. Effect of culture conditions on androgen sensitivity of the human prostatic cancer cell line LNCaP. Prostate. 1993; 23 (3): 213-23. Epub 1993 Jan. 1. doi: 10.1002/pros.2990230304. PubMed PMID: 7694266.
47. Ben-Hamo R, Berger A, Gavert N, Miller M, Pines G, Oren R, Pikarsky E, Benes C, Neuman T, Zwang Y, Efroni S, Getz G, Straussman R. Predicting and affecting response to cancer therapy based on pathway level biomarkers. Nature Commun. 2020; 11:3296. 10.1038/s41467-020-17090-y
48. Hey S, Franklin J, Avorn J, Kesselheim A. Success, failure, and transparency in biomarker-based drug development. Circulation. 2017; 10:e003121. Doi: 10.1161/circoutcomes. 116.003121.
49. Wendler A, Wehling M. Translatability scoring in drug development: eight case studies. J Transl Med. 2012; 10:39. Doi: 10.1186/1479-5876-10-39. PMID: 22397594
50. Hunter D, Losina E, Guermazi A, Burstein D, Lassere M, Kruas V. A pathway and approach to biomarker validation and qualification for osteoarthritis clinical trials. Curr Drug Targets. 2010; 11 (5): 536-545. Doi: doi: 10.2174/138945010791011947. PMID: 20199395
51. Cheraghchi-Bashi A, Parker C, Curry E, Salazar J, Gungor H, Saleem A, Cunnea P, Rama N, Salinas C, Mills G, Morris S, Kumar R, Gabra H, Stronach E. A putative biomarker signature for clinically effective AKT inhibition: correlation of in vitro, in vivo, and clinical data identifies the importance of modulation of the mTORC1 pathway. Oncotarget. 2015; 6 (39): 41736-49. Doi: 10.18632/oncotarget.6153 PMID: 26497682
52. Nielsen J. Systems Biology of Metabolism. Annu Rev Biochem. 2017; 86:245-75. Doi. 10.1146/annurev-biochem-061516-044757.
53. Rani V, Deep G, Singh R, Palle K, Yadav U. Oxidative stress and metabolic disorders: pathogenesis and therapeutic strategies. Life Sciences. 2016; 148:183-93. Doi: 10.1016/j.lfs.2016.02.002
54. Schmidt A, Forne I, Imhof A. Bioinformatic analysis of proteomics data. BMC Syst Biol. 2014; 8 Suppl 2:S3. Epub 2014 Jul. 18. doi: 10.1186/1752-0509-8-S2-S3. PubMed PMID: 25033288: PMCID: PMC4108846.
55. May M. Translating Big Data: The Proteomics Challenge. Science. 2018; 06.
56. Wordsworth S, Doble B, Payne K, Buchanan J, Marshall D A, McCabe C, Regier D A. Using “Big Data” in the Cost-Effectiveness Analysis of Next-Generation Sequencing Technologies: Challenges and Potential Solutions. Value Health. 2018; 21 (9): 1048-53. Epub 2018 Sep. 19. doi: 10.1016/j.jval.2018.06.016. PubMed PMID: 30224108.
57. Tools for Cell Line Identification. NIH/NIGMS: 2016-2019.
58. Masters J R, Thomson J A, Daly-Burns B, Reid Y A, Dirks W G, Packer P, Toji L H, Ohno T, Tanabe H, Arlett C F, Kelland L R, Harrison M, Virmani A, Ward T H, Ayres K L, Debenham P G. Short tandem repeat profiling provides an international reference standard for human cell lines. Proc Natl Acad Sci USA. 2001; 98 (14): 8012-7. Epub 2001 Jun. 21. doi: 10.1073/pnas. 121616198. PubMed PMID: 11416159; PMCID: PMC35459.
59. ATCC Products. In: ATCC, editor. Culture Method for PC-3 cells 2020.
60. Kaighn M E, Narayan K S, Ohnuki Y, Lechner J F, Jones L W. Establishment and characterization of a human prostatic carcinoma cell line (PC-3). Invest Urol. 1979; 17 (1): 16-23. Epub 1979 Jul. 1. PubMed PMID: 447482.
61. Probert C, Dottorini T, Speakman A, Hunt S, Nafee T, Fazeli A, Wood S, Brown J E, James V. Communication of prostate cancer cells with bone cells via extracellular vesicle RNA: a potential mechanism of metastasis. Oncogene. 2019; 38 (10): 1751-63. Epub 2018 Oct. 26. doi: 10.1038/s41388-018-0540-5. PubMed PMID: 30353168: PMCID: PMC6372071.
62. Geraghty R J, Capes-Davis A, Davis J M, Downward J, Freshney R I, Knezevic I, Lovell-Badge R, Masters J R, Meredith J, Stacey G N, Thraves P, Vias M, Cancer Research UK. Guidelines for the use of cell lines in biomedical research. Br J Cancer. 2014; 111 (6): 1021-46. Epub 2014 Aug. 15. doi: 10.1038/bjc.2014.166. PubMed PMID: 25117809: PMCID: PMC4453835.
63. Weaver I C. Epigenetic effects of glucocorticoids. Semin Fetal Neonatal Med. 2009; 14 (3): 143-50. Epub 2009 Feb. 17. doi: 10.1016/j.siny.2008.12.002. PubMed PMID: 19217839.
64. Claus J, Patel G, Autore F, Colomba A, Weitsman G, Soliman T N, Roberts S,
Zanetti-Domingues L C, Hirsch M, Collu F, George R, Ortiz-Zapater E, Barber P R, Vojnovic B, Yarden Y, Martin-Fernandez M L, Cameron A, Fraternali F, Ng T, Parker P J. Inhibitor-induced HER2-HER3 heterodimerisation promotes proliferation through a novel dimer interface. Elife. 2018; 7. Epub 2018 May 2. doi: 10.7554/eLife.32271. PubMed PMID: 29712619; PMCID: PMC5929906.
65. Xu Y, Xia J, Liu S, Stein S, Ramon C, Xi H, Wang L, Xiong X, Zhang L, He D, Yang W, Zhao X, Cheng X, Yang X, Wang H. Endocytosis and membrane receptor internalization: implication of F-BAR protein Carom. Front Biosci (Landmark Ed). 2017; 22:1439-57. Epub 2017 Feb. 16. doi: 10.2741/4552. PubMed PMID: 28199211: PMCID: PMC5315272.
66. Smith G P. Filamentous fusion phage: novel expression vectors that display cloned antigens on the virion surface. Science. 1985; 228 (4705): 1315-7. Epub 1985 Jun. 14. doi: 10.1126/science.4001944. PubMed PMID: 4001944.
67. Smith G P, Petrenko V A. Phage Display. Chem Rev. 1997; 97 (2): 391-410. Epub 1997 Apr. 1. doi: 10.1021/cr960065d. PubMed PMID: 11848876.
68. Dozmorov M G, Hurst R E, Culkin D J, Kropp B P, Frank M B, Osban J, Penning T M, Lin H K. Unique patterns of molecular profiling between human prostate cancer LNCaP and PC-3 cells. Prostate. 2009; 69 (10): 1077-90. Epub 2009 Apr. 4. doi: 10.1002/pros.20960. PubMed PMID: 19343732: PMCID: PMC2755240.
69. Sorvina A, Bader C A, Caporale C, Carter E A, Johnson I R D, Parkinson-Lawrence E J, Simpson P V, Wright P J, Stagni S, Lay P A, Massi M, Brooks D A, Plush S E. Lipid profiles of prostate cancer cells. Oncotarget. 2018; 9 (85): 35541-52. Epub 2018 Nov. 27. doi: 10.18632/oncotarget.26222. PubMed PMID: 30473749; PMCID: PMC6238979.
70. Kumar B, Koul S, Khandrika L, Meacham R B, Koul H K. Oxidative stress is inherent in prostate cancer cells and is required for aggressive phenotype. Cancer Res. 2008; 68 (6): 1777-85. Epub 2008 Mar. 15. doi: 10.1158/0008-5472.CAN-07-5259. PubMed PMID: 18339858.
71. Mahalingaiah P K, Singh K P. Chronic oxidative stress increases growth and tumorigenic potential of MCF-7 breast cancer cells. PLOS One. 2014; 9 (1): e87371. Epub 2014 Feb. 4. doi: 10.1371/journal.pone.0087371. PubMed PMID: 24489904: PMCID: PMC3905021.
72. Higgins L H, Withers H G, Garbens A, Love H D, Magnoni L, Hayward S W, Moyes C D. Hypoxia and the metabolic phenotype of prostate cancer cells. Biochim Biophys Acta. 2009; 1787 (12): 1433-43. Epub 2009 Jun. 16. doi: 10.1016/j.bbabio.2009.06.003. PubMed PMID: 19524545.
73. Costello L C, Feng P, Milon B, Tan M, Franklin R B. Role of zinc in the pathogenesis and treatment of prostate cancer: critical issues to resolve. Prostate Cancer Prostatic Dis. 2004; 7 (2): 111-7. Epub 2004 Jun. 4. doi: 10.1038/sj.pcan.4500712. PubMed PMID: 15175662: PMCID: PMC4464831.
74. Gao B, Lue H W, Podolak J, Fan S, Zhang Y, Serawat A, Alumkal J J, Fiehn O. Thomas G V. Multi-Omics Analyses Detail Metabolic Reprogramming in Lipids, Carnitines, and Use of Glycolytic Intermediates between Prostate Small Cell Neuroendocrine Carcinoma and Prostate Adenocarcinoma. Metabolites. 2019; 9 (5). Epub 2019 May 1. doi: 10.3390/metabo9050082. PubMed PMID: 31035489; PMCID: PMC6572715.
75. Costello L C, Franklin R B. The intermediary metabolism of the prostate: a key to understanding the pathogenesis and progression of prostate malignancy. Oncology. 2000; 59 (4): 269-82. Epub 2000 Nov. 30. doi: 10.1159/000012183. PubMed PMID: 11096338; PMCID: PMC4472372.
76. Zadra G, Photopoulos C, Loda M. The fat side of prostate cancer. Biochim Biophys Acta. 2013; 1831 (10): 1518-32. Epub 2013 Apr. 9. doi: 10.1016/j.bbalip.2013.03.010. PubMed PMID: 23562839: PMCID: PMC3766375.
77. Schlaepfer I R, Nambiar D K, Ramteke A, Kumar R. Dhar D, Agarwal C, Bergman B, Graner M. Maroni P, Singh R P. Agarwal R, Deep G. Hypoxia induces triglycerides accumulation in prostate cancer cells and extracellular vesicles supporting growth and invasiveness following reoxygenation. Oncotarget. 2015; 6 (26): 22836-56. Epub 2015 Jun. 19. doi: 10.18632/oncotarget.4479. PubMed PMID: 26087400; PMCID: PMC4673203.
78. Migita T, Okabe S, Ikeda K. Igarashi S, Sugawara S, Tomida A, Soga T, Taguchi R, Seimiya H. Inhibition of ATP citrate lyase induces triglyceride accumulation with altered fatty acid composition in cancer cells. Int J Cancer. 2014; 135 (1): 37-47. Epub 2013 Dec. 7. doi: 10.1002/ijc.28652. PubMed PMID: 24310723.
79. Teodoro J S, Palmeira C M, Rolo A P. Mitochondrial Membrane Potential (DeltaPsi) Fluctuations Associated with the Metabolic States of Mitochondria. Methods Mol Biol. 2018; 1782:109-19. Epub 2018 Jun. 1. doi: 10.1007/978-1-4939-7831-1_6. PubMed PMID: 29850996.
80. Zorova L D, Popkov V A, Plotnikov E Y, Silachev D N, Pevzner I B, Jankauskas S S, Babenko V A, Zorov S D, Balakireva A V, Juhaszova M, Sollott S J, Zorov D B. Mitochondrial membrane potential. Anal Biochem. 2018; 552:50-9. Epub 2017 Jul. 18. doi: 10.1016/j.ab.2017.07.009. PubMed PMID: 28711444: PMCID: PMC5792320.
81. Zhang B B, Wang D G, Guo F F, Xuan C. Mitochondrial membrane potential and reactive oxygen species in cancer stem cells. Fam Cancer. 2015; 14 (1): 19-23. Epub 2014 Oct. 1. doi: 10.1007/s10689-014-9757-9. PubMed PMID: 25266577.
82. Kuznetsov A V, Margreiter R, Amberger A, Saks V, Grimm M. Changes in mitochondrial redox state, membrane potential and calcium precede mitochondrial dysfunction in doxorubicin-induced cell death. Biochim Biophys Acta. 2011; 1813 (6): 1144-52. Epub 2011 Mar. 17. doi: 10.1016/j.bbamcr.2011.03.002. PubMed PMID: 21406203.
83. Schieber M. Chandel N S. ROS function in redox signaling and oxidative stress. Curr Biol. 2014; 24(10):R453-62. Epub 2014 May 23. doi: 10.1016/j.cub.2014.03.034. PubMed PMID: 24845678: PMCID: PMC4055301.
84. Thomas W D, Smith G P. The case for trypsin release of affinity-selected phages. Biotechniques. 2010; 49 (3): 651-4. Epub 2010 Sep. 22. doi: 10.2144/000113489. PubMed PMID: 20854266.
85. Brockhausen I. Pathways of O-glycan biosynthesis in cancer cells. Biochim Biophys Acta. 1999; 1473 (1): 67-95. Epub 1999 Dec. 2. doi: 10.1016/s0304-4165 (99) 00170-1. PubMed PMID: 10580130.
86. Brockhausen I. Mucin-type O-glycans in human colon and breast cancer: glycodynamics and functions. EMBO Rep. 2006; 7 (6): 599-604. Epub 2006 Jun. 3. doi: 10.1038/sj.embor.7400705. PubMed PMID: 16741504: PMCID: PMC1479595.
87. Brockhausen I. Yang J M. Burchell J. Whitehouse C. Taylor-Papadimitriou J. Mechanisms underlying aberrant glycosylation of MUC1 mucin in breast cancer cells. Eur J Biochem. 1995; 233 (2): 607-17. Epub 1995 Oct. 15. doi: 10.1111/j. 1432-1033.1995.607_2.x. PubMed PMID: 7588808.
88. Hanisch F G, Baldus S E. The Thomsen-Friedenreich (TF) antigen: a critical review on the structural, biosynthetic and histochemical aspects of a pancarcinoma-associated antigen. Histol Histopathol. 1997; 12 (1): 263-81. Epub 1997 Jan. 1. PubMed PMID: 9046061.
89. Goletz S, Cao Y, Danielczyk A, Ravn P, Schoeber U, Karsten U. Thomsen-Friedenreich antigen: the “hidden” tumor antigen. Adv Exp Med Biol. 2003; 535:147-62. Epub 2004 Jan. 13. doi: 10.1007/978-1-4615-0065-0_10. PubMed PMID: 14714894.
90. Russo C L, Spurr-Michaud S, Tisdale A. Pudney J, Anderson D, Gipson I K. Mucin gene expression in human male urogenital tract epithelia. Hum Reprod. 2006; 21 (11): 2783-93. Epub 2006 Sep. 26. doi: 10.1093/humrep/del164. PubMed PMID: 16997931: PMCID: PMC2893033.
91. Brocke C, Kunz. H. Synthetic Tumor-Associated Glycopeptide Antigens from the Tandem Repeat Sequence of the Epithelial Mucin MUC4. Synthesis. 2004; 4:525-42. doi: 10.1055/s-2004-815962.
92. Liu J, Xu F, Li J, Jiang H. Overexpression of Cosmc suppresses cell migration and invasion in different subtypes of breast cancer cells via Tn and T glycans. Biosci Rep. 2020; 40 (6). Epub 2020 May 27. doi: 10.1042/BSR20191062. PubMed PMID: 32452513; PMCID: PMC7300286.
93. Nasarre P, Potiron V, Drabkin H, Roche J. Guidance molecules in lung cancer. Cell Adh Migr. 2010; 4 (1): 130-45. Epub 2010 Feb. 9. doi: 10.4161/cam.4.1.10882. PubMed PMID: 20139699; PMCID: PMC2852570.
94. Neufeld G, Sabag A D. Rabinovicz N, Kessler O. Semaphorins in angiogenesis and tumor progression. Cold Spring Harb Perspect Med. 2012; 2 (1):a006718. Epub 2012 Feb. 9. doi: 10.1101/cshperspect.a006718. PubMed PMID: 22315716: PMCID: PMC3253028.
95. Hota P K, Buck M. Plexin structures are coming: opportunities for multilevel investigations of semaphorin guidance receptors, their cell signaling mechanisms, and functions. Cell Mol Life Sci. 2012; 69 (22): 3765-805. Epub 2012 Jun. 30. doi: 10.1007/s00018-012-1019-0. PubMed PMID: 22744749.
96. Le A P, Huang Y, Pingle S C, Kesari S, Wang H, Yong R L, Zou H, Friedel R H. Plexin-B2 promotes invasive growth of malignant glioma. Oncotarget. 2015; 6 (9): 7293-304. Epub 2015 Mar. 13. doi: 10.18632/oncotarget.3421. PubMed PMID: 25762646; PMCID: PMC4466685.
97. Leung C, Liu Y, Lu X, Kim M, Drysdale T A, Feng Q. Rac1 Signaling Is Required for Anterior Second Heart Field Cellular Organization and Cardiac Outflow Tract Development. J Am Heart Assoc. 2015; 5 (1). Epub 2016 Jan. 2. doi: 10.1161/JAHA.115.002508. PubMed PMID: 26722124; PMCID: PMC4859369.
98. Taddei M L, Parri M, Angelucci A, Onnis B, Bianchini F, Giannoni E, Raugei G, Calorini L, Rucci N, Teti A, Bologna M, Chiarugi P. Kinase-dependent and -independent roles of EphA2 in the regulation of prostate cancer invasion and metastasis. Am J Pathol. 2009; 174 (4): 1492-503. Epub 2009 Mar. 7. doi: 10.2353/ajpath.2009.080473. PubMed PMID: 19264906: PMCID: PMC2671379.
99. Segawa Y, Yoshimura R, Hase T, Nakatani T, Wada S, Kawahito Y, Kishimoto T, Sano H. Expression of peroxisome proliferator-activated receptor (PPAR) in human prostate cancer. Prostate. 2002; 51 (2): 108-16. Epub 2002 Apr. 12. doi: 10.1002/pros. 10058. PubMed PMID: 11948965.
100. Collett G P, Betts A M, Johnson M I, Pulimood A B. Cook S, Neal D E, Robson C N. Peroxisome proliferator activated receptor alpha is an androgen-responsive gene in human prostate and is highly expressed in prostatic adenocarcinoma. Clin Cancer Res. 2000; 6 (8): 3241-8. Epub 2000 Aug. 24. PubMed PMID: 10955810.
101. Okumoto K, Tamura S, Honsho M, Fujiki Y. Peroxisome: Metabolic Functions and Biogenesis. Adv Exp Med Biol. 2020; 1299:3-17. Epub 2021 Jan. 9. doi: 10.1007/978-3-030-60204-8_1. PubMed PMID: 33417203.
102. Chen J. Chen J. Fu H. Li Y. Wang L. Luo S. Lu H. Hypoxia exacerbates nonalcoholic fatty liver disease via the HIF-2alpha/PPARalpha pathway. Am J Physiol Endocrinol Metab. 2019; 317 (4): E710-E22. Epub 2019 Aug. 21. doi: 10.1152/ajpendo.00052.2019. PubMed PMID: 31430204.
103. Thompson J, Lepikhova T, Teixido-Travesa N, Whitehead M A. Palvimo J J, Janne O A. Small carboxylterminal domain phosphatase 2 attenuates androgen-dependent transcription. EMBO J. 2006; 25 (12): 2757-67. Epub 2006 May 26. doi: 10.1038/sj.emboj. 7601161. PubMed PMID: 16724108: PMCID: PMC1500849.
104. Zha S, Ferdinandusse S, Hicks J L, Denis S, Dunn T A. Wanders R J, Luo J, De Marzo A M, Isaacs W B. Peroxisomal branched chain fatty acid beta-oxidation pathway is upregulated in prostate cancer. Prostate. 2005; 63 (4): 316-23. Epub 2004 Dec. 16. doi: 10.1002/pros.20177. PubMed PMID: 15599942.
105. Landon L A. Zou J. Deutscher S L. Effective combinatorial strategy to increase affinity of carbohydrate binding by peptides. Mol Divers. 2004; 8 (1): 35-50. Epub 2004 Feb. 18. doi: 10.1023/b:modi.0000006897.40575.41. PubMed PMID: 14964786.
106. Smith G P. Smith Lab Homepage University of Missouri, Department of Biology. Available from: www.biosci.missouri.edu/smithgp/.
107. Newton-Northup J, Deutscher S. Contending with target unrelated peptides from phage display. J Mol Imaging Dynam. 2012; 2 (2). DOI: 10.4172/2155-9937.1000e101
108. Northup J, Deutscher, S. Cytotoxic tumor-targeting peptides from in vivo phage display. Comb Chem High Throughput Screen. 2016; 19 (5): 370-7. Doi: 10.2174/1386207319666160408151423
109. Newton J R, Kelly K A, Mahmood U, Weissleder R, Deutscher S L. In vivo selection of phage for the optical imaging of PC-3 human prostate carcinoma in mice. Neoplasia. 2006; 8 (9): 772-80. Epub 2006 Sep. 21. doi: 10.1593/neo.06331. PubMed PMID: 16984734: PMCID: PMC1584300.
110. Newton-Northup J R, Dickerson M T, Kumar S R, Smith G P, Quinn T P, Deutscher S L. In vivo bacteriophage peptide display to tailor pharmacokinetics of biological nanoparticles. Mol Imaging Biol. 2014; 16 (6): 854-64. Epub 2014 Jul. 9. doi: 10.1007/s11307-014-0762-z. PubMed PMID: 25001195.
111. Newton-Northup J R, Figueroa S D, Deutscher S L. Streamlined in vivo selection and screening of human prostate carcinoma avid phage particles for development of peptide based in vivo tumor imaging agents. Comb Chem High Throughput Screen. 2011; 14 (1): 9-21. Epub 2010 Oct. 21. doi: 10.2174/1386207311107010009. PubMed PMID: 20958260.
112. Newton-Northup J R, Figueroa S D, Quinn T P, Deutscher S L. Bifunctional phage-based pretargeted imaging of human prostate carcinoma. Nucl Med Biol. 2009; 36 (7): 789-800. Epub 2009 Sep. 2. doi: 10.1016/j.nucmedbio.2009.04.010. PubMed PMID: 19720291: PMCID: PMC2739128.
113. Smith G P, Gingrich T R. Hydroxyapatite chromatography of phage-display virions. Biotechniques. 2005; 39 (6): 879-84. Epub 2005 Dec. 31. doi: 10.2144/000112032. PubMed PMID: 16382907.
114. Grimes T, Potter S S, Datta S. Integrating gene regulatory pathways into differential network analysis of gene expression data. Sci Rep. 2019; 9 (1): 5479. Epub 2019 Apr. 4. doi: 10.1038/s41598-019-41918-3. PubMed PMID: 30940863: PMCID: PMC6445151.
115. Wei P, Pan W. Incorporating gene networks into statistical tests for genomic data via a spatially correlated mixture model. Bioinformatics. 2008; 24 (3): 404-11. doi: 10.1093/bioinformatics/btm612. PubMed PMID: WOS: 000252903700014.
116. Dheda K, Huggett J F, Bustin S A, Johnson M A, Rook G, Zumla A. Validation of housekeeping genes for normalizing RNA expression in real-time PCR. Biotechniques. 2004; 37 (1): 112-4, 6, 8-9. Epub 2004 Jul. 31. doi: 10.2144/04371RR03. PubMed PMID: 15283208. Contact PD/PI: Northup, Jessica R References Cited
117. Townsend M H, Felsted A M, Ence Z E, Piccolo S R, Robison R A, O'Neill K L. Falling from grace: HPRT is not suitable as an endogenous control for cancer-related studies. Mol Cell Oncol. 2019; 6 (2): 1575691. Epub 2019 May 28. doi: 10.1080/23723556.2019.1575691. PubMed PMID: 31131300: PMCID: PMC6512905.

### Example 5

In addition to the studies described in Example 4, studies described in this example show that various of the isolated clones bind to cells differentially based on species of origin (e.g., mouse versus human), tissue and/or cell type.

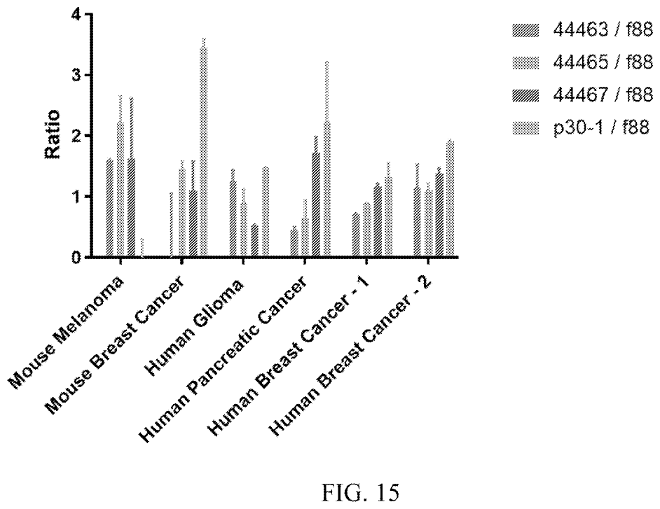

To illustrate changes of cell surface binding of phage clones 44463, 44465 and 44467 due to differences in targeted biomarker expression levels between different cell lines, previous cell binding experiments were repeated using a variety of different cell lines. FIG. 15 shows differences of phage clone binding as a measure of biomarker expression levels in cultured mouse cell lines versus human cell lines, as well as differences between biomarker expression levels between different tissue types. These data indicate that though these phage-targeted biomarkers were selected based upon human prostate carcinoma cell line, LNCaP, the biomarkers are useful for phenotypic characterization of many cell types. The data also indicate that the changes in expression levels across multiple mammalian cell lines can be used to characterize and identify these different cell lines.

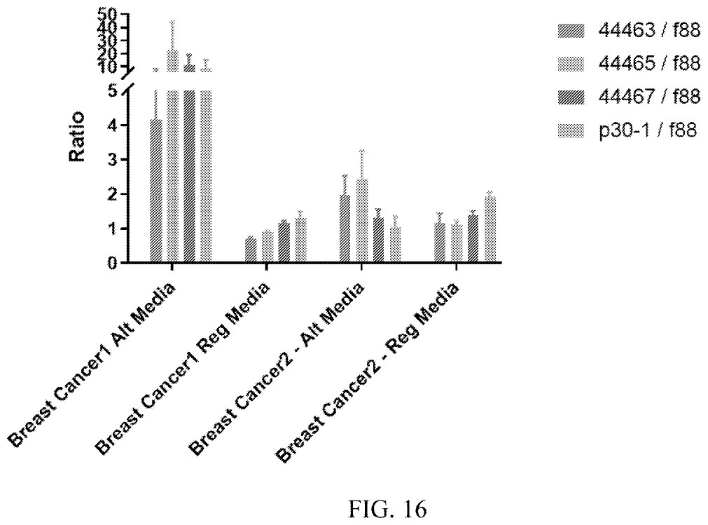

To further illustrate the sensitivity of cultured mammalian cells to their environment, the two human breast carcinoma cell lines used in the experiments above (i.e., Human Breast Cancer-1, Human Breast Cancer-2) were propagated in two different media compositions described in the literature. The cell lines were defrosted and maintained within the different media for two to three passages. Subsequently, the cells were processed as described earlier and binding of the phage clones to the cell surfaces was tested. The data (FIG. 16) showed significant differences in phage binding due to changes in biomarker expression levels on the cell surfaces.

The results are illustrated in FIGS. 15 and 16 and show the utility the phage clones for probing biomarker expression levels. From these data and the data in Example 4, binding of these phage clones to their biomarkers has been shown to have utility in probing some common cell culturing variables, including cell passage number (i.e., age), confluency and the type of media in which cells are propagated. The data also demonstrate the utility of these phage clones to probe cell/tissue type and the species from which cells originate.

### Example 6

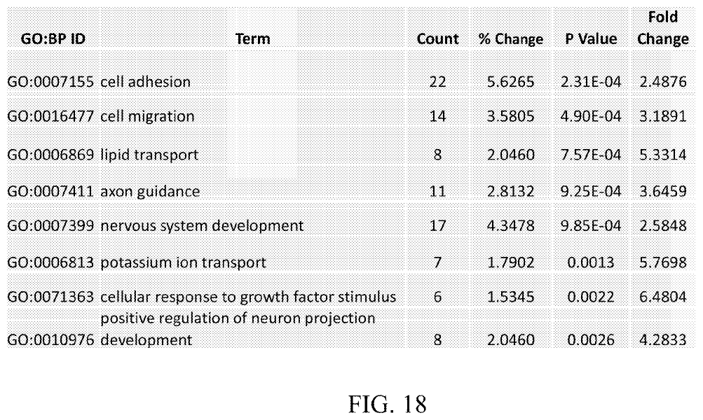

To examine possible interrelationships between our phage clones and known genes related to the parameters we've investigated (i.e., cell age), an RNAseq analysis was performed on LNCaP cells at passage number 10 (young cells) and at a passage number of about 40 (old cells). The transcriptiomes of the young and old LNCaP cells were then compared according to expression level and using pathway analysis (FIG. 18).

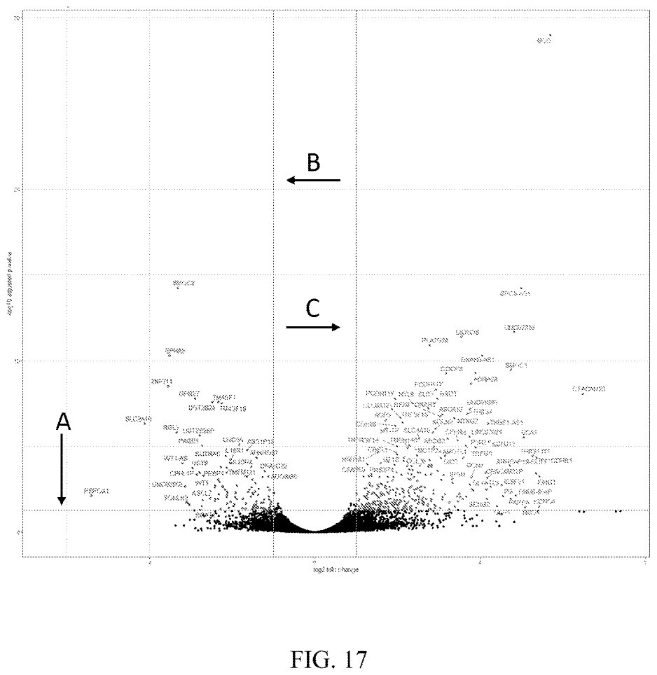

As shown in FIG. 17, expression of 455 genes was different between young and old cells (i.e., genes indicated by the points above the horizontal line labeled “A”). Differences in expression were determined by comparing the number of reads between young and old cells based on number of reads for each gene in the two libraries. Three hundred fifty-five (355) of those genes were increased in expression (i.e., genes indicated by the points to the left of the vertical line labeled “B”). One hundred eighteen (118) of those genes were decreased in expression (i.e., genes indicated by the points to the right of the vertical line labeled “C”).

These genes were then assigned functions using the gene ontology database (geneontology.org). The genetic pathways in which the genes play a role were identified. Each identified genetic pathway has a Gene Ontology Biological Process Identification Number (GO BP ID in FIG. 18) which was then further analyzed for differential expression levels.

The genetic pathways identified are involved in cell adhesion, migration, lipid transport, and other functions (FIG. 18). The table shows those genetic pathways where differences in expression of the pathways between young and old cells FIG. 18 shows pathways where differences in expression levels of the pathways in young as compared to old cells were different at a p-value of 0.0026 or below: In FIG. 18, the column labeled “Count” is the number of genes within the particular GO BP ID pathway that showed changes in expression. The column labeled “% Change” indicates the percentage of genes in a pathway whose expression was changed compared to the total number of genes in the pathway. The column labeled “Fold Change” is how much a particular pathway is enriched using the given input gene list.

For example, there was an approximately 3.6-fold change in expression of genes involved in axon guidance and an approximately 3.2-fold change in expression of genes involved in cell migration in the older cells. Coincidentially, the CRISPR knockout studies described in Example 4 showed that knockout of genes involved in semaphoring, plexin, ephrin/eph receptor neuronal guidance system/axon outgrowth pathways reduced or prevented phage clone 44463 from binding to the LNCaP cells.

Additionally, FIG. 18 indicates an approximately 5.3-fold change in expression of genes involved in lipid transport in the older cells. Coincidentally, the CRISPR knockout studies described in Example 4 showed that knockout of genes involved in lipid transport reduced or prevented phage clone 44465 from binding to LNCaP cells.

Described herein is characterization of phage clones that can quantify cell surface biomarkers related to cell type and passage number (i.e., age), and provide a readout of appropriate versus inappropriate culture conditions, including confluency and media composition. Two of these phage-targeted cell surface biomarkers were studied by using CRISPR knockout libraries to knockout genes encoding these biomarkers and to identify genes whose expression was changed in cells containing the gene knockouts. A short list of functionally-related genes was identified for both the 44463 and 44465 clones. The 44463 gene is predicted to target a semaphorin, plexin, or ephrin/eph receptor within the neuronal guidance system/axon outgrowth pathways. The 44465 gene is predicted to target a lipid involved in the peroxisome and/or lipid transport system. Additionally, the data shown in FIG. 18 show that both the axon guidance and lipid transport systems were identified as pathways with significant changes in expression in old versus new cells. As such, the repeated identification of these pathways strengthens our claims as to the identity of the biomarkers.

Further, we have illustrated the principles of our focused interrogation of three tiers of biology. First, we identified biomarkers with targeting phage clones for the analysis of protein/lipid/etc cell surface levels. Second, we illustrated the utility of probing changes of RNA transcript expression levels within cells. Third, all of this used STR-verified cell lines.

### EQUIVALENTS

Those skilled in the art will recognize, or be able to ascertain, using no more than routine experimentation, numerous equivalents to the specific substances and procedures described herein. Such equivalents are considered to be within the scope of this invention.

1-14. (canceled)

15. A method for quantifying an analyte on a surface of a cell, comprising:
a. contacting a bacteriophage comprising a peptide and a nucleic acid with the cell, wherein the peptide can bind specifically to the analyte, to form a complex between the bacteriophage and the analyte; and
b. quantifying an amount of the analyte in the complex by measuring an amount of the nucleic acid in the complex.

16. The method of claim 15, wherein the amount of the nucleic acid is measured by polymerase chain reaction (PCR).

17. The method of claim 16, wherein the PCR comprises quantitative polymerase chain reaction (qPCR).

18-19. (canceled)

20. The method of claim 15, wherein the genome associated with the bacteriophage comprises a viral genome or a phagemid.

21. The method of claim 15, wherein the bacteriophage comprises a filamentous bacteriophage having a DNA genome genetically modified to express the peptide.

22-26. (canceled)

27. The method of claim 15, wherein the analyte comprises a protein, lipid, carbohydrate, nucleic acid, or combinations thereof

28. The method of claim 15, wherein the peptide that binds the analyte is selected from the group consisting of:
(SEQ ID NO: 1)
a. AVAGLFTGPQVDTVV;
(SEQ ID NO: 2)
b. HHFLFPSFVWAVAYS;
(SEQ ID NO: 3)
c. YYVGFGPLRVVRSVE;
(SEQ ID NO: 4)
d. TSRASWCCAVVVDSL;
and
(SEQ ID NO: 5)
e. AGATGYRYGSPKTRF.

29. The method of claim 15, additionally comprising quantifying an amount of an mRNA encoding the analyte in the cell.

30. The method of claim 29, wherein the mRNA is quantified using polymerase chain reaction (PCR).

31-35. (canceled)

36. The method of claim 15, wherein the method is used in combination with analyzing the cell using short tandem repeat (STR) profiling.

37-62. (canceled)

63. A bacteriophage or phagemid particle having a genome encoding a peptide fused to a coat protein of the bacteriophage or phagemid particle, the peptide comprising an amino acid sequence at least 95% identical to:
(SEQ ID NO: 1)
a. AVAGLFTGPQVDTVV;
(SEQ ID NO: 2)
b. HHFLFPSFVWAVAYS;
(SEQ ID NO: 3)
c. YYVGFGPLRVVRSVE;
(SEQ ID NO: 4)
d. TSRASWCCAVVVDSL;
or
(SEQ ID NO: 5)
e. AGATGYRYGSPKTRF.

64. A kit for evaluating cell health, comprising:
a bacteriophage or genome therefrom that encodes a peptide for use in the method of claim 15.

65-75. (canceled)

76. The method of claim 15 wherein the cell grows in suspension or attached to a surface.

77. The method of claim 15, wherein the analyte comprises a cell surface receptor, a cell surface ligand, a cluster of differentiation (CD) molecule, an MHC molecule, or a tumor antigen.

78. The method of claim 15, wherein an amount of the analyte on the surface of the cell is dependent on a measure of cell health (MCH), comprising:
a. plating efficiency
b. cell growth or proliferation rate;
c. cell viability;
d. cellular metabolism;
e. oxygen consumption and/or extracellular acidification rate;
f. reactive oxygen species (ROS);
g. mitochondrial membrane potential; or
h. combinations thereof.

79. The method of claim 15, wherein an amount of the analyte on the surface of the cell is dependent on:
a. cell identity;
b. tissue type from which the cell is derived;
c. cell age;
d. cell passage number;
e. cell density
f. composition of media in which the cell is grown;
g. culture conditions; or
h. combinations thereof.

80. A method for monitoring cellular health or phenotype, comprising:
contacting a cell with a bacteriophage having a peptide displayed on a surface of the bacteriophage, the peptide capable of binding to the cell based on cell identity, tissue type from which the cell is derived, cell age, cell passage number, cell density, composition of media in which the cell is grown, or cell culture conditions; and
measuring an amount of nucleic acid from genomes of bacteriophage bound to the cell to determine binding of the peptide to the cell.


SEQUENCE LISTING (9 sequences)

SEQ ID NO: 1 - protein, 15 aa, synthetic construct
AVAGLFTGPQVDTVV

SEQ ID NO: 2 - protein, 15 aa, synthetic construct
HHFLFPSFVWAVAYS

SEQ ID NO: 3 - protein, 15 aa, synthetic construct
YYVGFGPLRVVRSVE

SEQ ID NO: 4 - protein, 15 aa, synthetic construct
TSRASWCCAVVVDSL

SEQ ID NO: 5 - protein, 15 aa, synthetic construct
AGATGYRYGSPKTRF

SEQ ID NO: 6 - DNA, 18 bp, synthetic construct
  source 1..18: mol_type = other DNA
tgaattttctgtatgagg

SEQ ID NO: 7 - DNA, 20 bp, synthetic construct
  source 1..20: mol_type = other DNA
cgttacgctaactatgaggg

SEQ ID NO: 8 - DNA, 20 bp, synthetic construct
  source 1..20: mol_type = other DNA
ttttggagattttcaacgtg

SEQ ID NO: 9 - protein, 6 aa, synthetic construct
KCCYSL